SEQUENCE LISTING (196 sequences)

SEQ ID NO: 1 - DNA, 615 bp, Artificial Sequence
  Synthetic White Spot Syndrome Virus (WSSV) VP 28
atggatctttctttcactctttcggtcgtgtcggccatcctcgccatcactgctgtgattgctgtatttattgtgatttttaggtatcacaacactgtgaccaagaccatcgaaacccacacagacaatatcgagacaaacatggatgaaaacctccgcattcctgtgactgctgaggttggatcaggctacttcaagatgactgatgtgtcctttgacagcgacaccttgggcaaaatcaagatccgcaatggaaagtctgatgcacagatgaaggaagaagatgcggatcttgtcatcactcccgtggagggccgagcactcgaagtgactgtggggcagaatctcacctttgagggaacattcaaggtgtggaacaacacatcaagaaagatcaacatcactggtatgcagatggtgccaaagattaacccatcaaaggcctttgtcggtagctccaacacctcctccttcacccccgtctctattgatgaggatgaagttggcacctttgtgtgtggtaccacctttggcgcaccaattgcagctaccgccggtggaaatcttttcgacatgtacgtgcacgtcacctactctggcactgagaccgagtaa

SEQ ID NO: 2 - DNA, 510 bp, Artificial Sequence
  Synthetic Enterocytozoon hepatopenaei (EHP) SSU (18srRNA)
gcctgagagatggctcccacgtccaaggatggcagcaggcgcgaaaattgtccactcttttgagaggagacagttatgaaacgtgagtagaagggtcgagtgtaaaaaccttgacgtgaagcaattggagggcaagttttggtgccagcagccgcggtaattccaactccaagagtgtctatggtggatgctgcagttaaagggtccgtagtcgtagatgcaattaaaaggtggtgttaaaagccattgagtttgttgagagtagcggaacggatagggagcatggtataggtgggcaaagaatgaaatctcaagaccccacctggaccaacggaggcgaaagcgatgctcttagacgtatctggggatcaaggacgaaggctagagtatcgaaagtgattagacaccgctgtagttctagcagtaaactatgccgacaatgctgggtgttgcgagagcgatgcttggtgtgggagaaatcttagttttcgggctctggggatagtacgc

SEQ ID NO: 3 - DNA, 614 bp, Artificial Sequence
  Synthetic Necrotising Hepatopancreatitis/Hepatobacter penaei (NHP-B) 16SrRNA
cgttggaggttcgtccttcagtgtcgcagctaacgcgtgaagcattccgcctggggagtacggtcgcaagattaaaactcaaaggaattgacgggggcctgcacaagcggtggagcatgtggtttaattcgacgcaacgcgcaaaaccttaccagcttttgacatagggacagaaggcatcagagatgaagcctgcggttcggccgggtcccgtacaggtgctgcatggctgtcgtcagctcgtgtcgtgagatgttgggttaagtcccgcaacgagcgcaaccctcgttcttagttgccagcagttcggctggggactctaagaaaactgccggtgacaagccggaggaaggtggggatgatgtcaggtcctcatggcccttatgggctgggctacacacgtgctacaatggcgatcacaatgagaagcaagggggtgacccggagccaatctctaaaaatcgtctcagttcggattgttctctgcaactcgagagcatgaagttggaatcgctagtaatcgcagatcagcatgctgcggtgaatacgttcacgggccttgtacacaccgcccgtcaagccatggaagttatcttttctttaaggcgatgagc

SEQ ID NO: 4 - DNA, 333 bp, Artificial Sequence
  Synthetic Necrotising Hepatopancreatitis/Hepatobacter penaei (NHP-B) Flg E
aggcaaacaaacaacccttggtaacacatggaacaccctgtctccccaattcacgccccaaagcatgccgcccaaggtgtttgtccaaggctggctcaacagcgaaggggtgccggtggggtccgctgacagcaccattgccctggatcccagcaccatggctctttctggcaaccaatatcaactgcaaacgcccgagcaagatgggcgtatggccgcccctttcaaaggcattagcatcagcgataccggggtgatttcttctgaattttttggtcaagaccccaaacctgaatggcagttgtttttggtgaactttcccaacaacaacgc

SEQ ID NO: 5 - DNA, 991 bp, Artificial Sequence
  Synthetic Necrotising Hepatopancreatitis/Hepatobacter penaei (NHP-B) Gyr B
gcaaacgccccggcatgtatatcggcaacaccgaagatggctcgggcctacatcacatggtgtatgaggtggtggacaacgcggtggatgaggcgctggccggtcattgcacacgcattcatgtgacgcttcaccccaatggctctgtcacggtaaaagatgacggccgcggcgtgcctgtggacattcatgcggaagaaggcgtgtccgcagcccaggtcatcatgacccacctacatgcagggggtaagtttgatgaaaacagctataaggtgtcaggcgggctgcacggcgtgggtgtgtcggtggtcaacgcgctgtccacgtggctggagatgcgcatttggcgggccggcaaagaacatgttatacgatttgaaaacggtgagcccgtggccccgttggcggtggtgggcgatgcggaaccggggcaaaccggcacacaagtcacctttttgccttcgcccaagacctttcagattaccaaattttcttttgacaccttagaaaatcgctttcgcgagcttgcgtttttgaaccccggcatcaccattgtgctgacggataagcgggatgagcctaaggaaagcaccctgcattatgagggcggtgtggcttcgtttgtggcctatctgaatcgcgcgcgcacggccctgcacacgccttgcatccaccttcatggtgtgaaagacgatattgtgctggatgtggcgatggaatggaccgacacctatcatgaaagcctcttgtgctttaccaacaacattccccagcgtgacggcggcacccacttgtcagggcttaaggcatctcttacccgggtggtgggtggctttgtggccaaaagcaatattcctaaaaaagacaaggtggccaccacgggcgaagatgtgcgcgaagggctgacgtgcgtgttgtcggtaaaggtgccggatccgaaattttcgtctcagaccaaagacaagttggtgtcttctgaggtgcgcagcgt

SEQ ID NO: 6 - DNA, 900 bp, Artificial Sequence
  Synthetic Infectious Hypodermal and Hematopoietic Necrosis Virus (IHHNV) Capsid protein
catttgagactctcacatttacagacaccccatatttagaaatatttaaggatactactggactacataatcaactatcaactaaggaagccgacgtaacattggcaaagtggatacaaaatccccaacttgtgaccgtacaatcaacagcagcaaactatgaagacccaatccaacaatttggattcatggaacaaatgcgaaccggtgacagaaaagcctatacaatccatggtgacactagaaattggtatggcggagaaataccaacaaccggacccaccttcatcccaaaatggggtggtcaattaaaatgggacaaaccatcccttggaaacttagtctacccagcagaccaccatacaaacgactggcaacagatcttcatgagaatgtcaccaatcaaaggaccaaatggagacgaacttaaacttggctgcagagtacaagccgacttcttcctacacctagaagtacgactcccaccacaaggatgtgtcgcaagtttggggatgttacaatatcttcacgcaccatgtactggacagcttaacaaatgttatattatgcatactaactaaatatattcgatgtgcaatatatacccgattatatccagcttttaccaataaacatatatagcttattatcatctatacctaccctatatacataccagctacccaggcaaggtgggactccggctacccaggcatggtgggacacttttcttctattgacgacatatttcgtcacttacgtcacttacaaaagactaaaatccctatcgtcagtcagtcatttagagtcagggatattgtccgccgtcacttagagcgcgaagcgcgagtatccatcatttaaattagtggtatgacgtcacatattaagttaacggtt

SEQ ID NO: 7 - DNA, 240 bp, Artificial Sequence
  Synthetic Infectious Myonecrosis Virus (IMNV) ORF 1
gattttgtaaatgggacccaacctacattgaaatttctgattgtatgctgccacctccagaccttacatcgtgcggggacgttgagagtaatcctggacctatcatacatagcgttgcatttgcaagaactggttcagtatggacacctgccacctttactttcaatactacatcatccccgggtagactgcaagtacaaatgtcatccagcgacaatagatatgggttcaattctgttt

SEQ ID NO: 8 - DNA, 851 bp, Artificial Sequence
  Synthetic Penaeus spp. 18SrRNA
tgccttatcagctctcgattgtaggttaaacgcctacaatggctatcacgggtaacggggaatcagggttcgattccggagagggagcctgagaaacggctaccacatctaaggaaggcagcaggcgcgcaaattacccactcccggcacggggaggtagtgacgaaaaatactgttgcgagccccgaacggggcctcgcaattggaatgagtacactttaaatccttgtacgaggatcgagtggagggcaagtctggtgccagccgccgcggtaattccagctccactagcgtatattaaagttgttgcggttgaaacgctcgtagtttgacttctgctccggaccggcggtccgccttagcggcggctactgccgggttccgagctgtgtccccgccggcgcacatggggtttttatgcccttaaccgggtgtccccttgtggccggcacgtttactttgaaaaaattagagtgctcagagcaggctggtttttgcttacagcccgaatggtcgtgcatggaatgatggaacaggacctcggttctattttgtcggtttttcggaacccgaggtaatgattaatagaagcagacgggggcattcgtactgcgacgctagaggtgaaattcttagaccgtcgcatgacgacctactgcgaaagcatctgaggatgttttcattgatcaagaacgaaagttagaggttcgaaggcgatcagataccgccctagttctaaccttaaacgatgctgactagcgatccgccgcagttattcccatgacccggcgggtagcttccgggaaaccaaagtctttgagttccgggggaagtatggttgcaaagctgaa

SEQ ID NO: 9 - DNA, 20 bp, Artificial Sequence
  Synthetic Whate Spot Syndrome Virus (WSSV) VP28 primer F3
tggtgccaaagattaaccca

SEQ ID NO: 10 - DNA, 20 bp, Artificial Sequence
  Synthetic Whate Spot Syndrome Virus (WSSV) VP28 primer B3
ggtctcagtgccagagtagg

SEQ ID NO: 11 - DNA, 42 bp, Artificial Sequence
  Synthetic Whate Spot Syndrome Virus (WSSV) VP28 primer FIP
accacacacaaaggtgccaactctttgtcggtagctccaaca

SEQ ID NO: 12 - DNA, 38 bp, Artificial Sequence
  Synthetic Whate Spot Syndrome Virus (WSSV) VP28 primer BIP
accacctttggcgcaccaatcgtgcacgtacatgtcga

SEQ ID NO: 13 - DNA, 22 bp, Artificial Sequence
  Synthetic Whate Spot Syndrome Virus (WSSV) VP28 primer LF
tcatcctcatcaatagagacgg

SEQ ID NO: 14 - DNA, 17 bp, Artificial Sequence
  Synthetic Whate Spot Syndrome Virus (WSSV) VP28 primer LB
agctaccgccggtggaa

SEQ ID NO: 15 - DNA, 25 bp, Artificial Sequence
  Synthetic Whate Spot Syndrome Virus (WSSV) VP28 F primer
gcgaagcttaatggatctttctttc

SEQ ID NO: 16 - DNA, 25 bp, Artificial Sequence
  Synthetic Whate Spot Syndrome Virus (WSSV) VP28 R primer
gacacatctagatactcggtctcag

SEQ ID NO: 17 - DNA, 20 bp, Artificial Sequence
  Synthetic White Spot Syndrome Virus (WSSV) VP28 primer B3
ggtctcagtgccagagtagg

SEQ ID NO: 18 - DNA, 19 bp, Artificial Sequence
  Synthetic White Spot Syndrome Virus (WSSV) VP28 primer BIP1
accacctttggcgcaccaa

SEQ ID NO: 19 - DNA, 19 bp, Artificial Sequence
  Synthetic White Spot Syndrome Virus (WSSV) VP28 primer BIP2
cgtgcacgtacatgtcgat

SEQ ID NO: 20 - DNA, 20 bp, Artificial Sequence
  Synthetic White Spot Syndrome Virus (WSSV) VP28 primer F3
tggtgccaaagattaaccca

SEQ ID NO: 21 - DNA, 22 bp, Artificial Sequence
  Synthetic White Spot Syndrome Virus (WSSV) VP28 primer FIP 1
accacacacaaaggtgccaact

SEQ ID NO: 22 - DNA, 20 bp, Artificial Sequence
  Synthetic White Spot Syndrome Virus (WSSV) VP28 primer FIP2
ctttgtcggtagctccaaca

SEQ ID NO: 23 - DNA, 17 bp, Artificial Sequence
  Synthetic White Spot Syndrome Virus (WSSV) VP28 primer LB
agctaccgccggtggaa

SEQ ID NO: 24 - DNA, 22 bp, Artificial Sequence
  Synthetic White Spot Syndrome Virus (WSSV) VP28 primer LF
tcatcctcatcaatagagacgg

SEQ ID NO: 25 - DNA, 18 bp, Artificial Sequence
  Synthetic Enterocytozoon hepatopenaei (EHP) SSU 18srRNA primer B3
gagcatcgctttcgcctc

SEQ ID NO: 26 - DNA, 22 bp, Artificial Sequence
  Synthetic Enterocytozoon hepatopenaei (EHP) SSU 18srRNA primer BIP1
agtagcggaacggatagggagc

SEQ ID NO: 27 - DNA, 19 bp, Artificial Sequence
  Synthetic Enterocytozoon hepatopenaei (EHP) SSU 18srRNA primer BIP2
ccaggtggggtcttgagat

SEQ ID NO: 28 - DNA, 19 bp, Artificial Sequence
  Synthetic Enterocytozoon hepatopenaei (EHP) SSU 18srRNA primer F3
tggagggcaagttttggtg

SEQ ID NO: 29 - DNA, 21 bp, Artificial Sequence
  Synthetic Enterocytozoon hepatopenaei (EHP) SSU 18srRNA primer FIP1
acggaccctttaactgcagca

SEQ ID NO: 30 - DNA, 18 bp, Artificial Sequence
  Synthetic Enterocytozoon hepatopenaei (EHP) SSU 18srRNA primer FIP 2
cagccgcggtaattccaa

SEQ ID NO: 31 - DNA, 17 bp, Artificial Sequence
  Synthetic Enterocytozoon hepatopenaei (EHP) SSU 18srRNA primer LB
atggtataggtgggcaa

SEQ ID NO: 32 - DNA, 19 bp, Artificial Sequence
  Synthetic Enterocytozoon hepatopenaei (EHP) SSU 18srRNA primer LF
accatagacactcttggag

SEQ ID NO: 33 - DNA, 19 bp, Artificial Sequence
  Synthetic Necrotising Hepatopancreatitis/Hepatobacter penaei (NHP-B) 16SrRNA 1 primer B3
agcatgctgatctgcgatt

SEQ ID NO: 34 - DNA, 22 bp, Artificial Sequence
  Synthetic Necrotising Hepatopancreatitis/Hepatobacter penaei (NHP-B) 16SrRNA 1 primer BIP1
aatgagaagcaagggggtgacc

SEQ ID NO: 35 - DNA, 20 bp, Artificial Sequence
  Synthetic Necrotising Hepatopancreatitis/Hepatobacter penaei (NHP-B) 16SrRNA 1 primer BIP2
gctctcgagttgcagagaac

SEQ ID NO: 36 - DNA, 18 bp, Artificial Sequence
  Synthetic Necrotising Hepatopancreatitis/Hepatobacter penaei (NHP-B) 16SrRNA 1 primer F3
aaactgccggtgacaagc

SEQ ID NO: 37 - DNA, 20 bp, Artificial Sequence
  Synthetic Necrotising Hepatopancreatitis/Hepatobacter penaei (NHP-B) 16SrRNA 1 primer FIP 1
tagcacgtgtgtagcccagc

SEQ ID NO: 38 - DNA, 18 bp, Artificial Sequence
  Synthetic Necrotising Hepatopancreatitis/Hepatobacter penaei (NHP-B) 16SrRNA 1 primer FIP 2
ggaggaaggtggggatga

SEQ ID NO: 39 - DNA, 22 bp, Artificial Sequence
  Synthetic Necrotising Hepatopancreatitis/Hepatobacter penaei (NHP-B) 16SrRNA 1 primer LB
cggagccaatctctaaaaatcg

SEQ ID NO: 40 - DNA, 18 bp, Artificial Sequence
  Synthetic Necrotising Hepatopancreatitis/Hepatobacter penaei (NHP-B) 16SrRNA 1 primer LF
ggccatgaggacctgaca

SEQ ID NO: 41 - DNA, 18 bp, Artificial Sequence
  Synthetic Necrotising Hepatopancreatitis/Hepatobacter penaei (NHP-B) 16SrRNA 2 primer B3
cttagagtccccagccga

SEQ ID NO: 42 - DNA, 20 bp, Artificial Sequence
  Synthetic Necrotising Hepatopancreatitis/Hepatobacter penaei (NHP-B) 16SrRNA 2 primer BIP 1
ggctgtcgtcagctcgtgtc

SEQ ID NO: 43 - DNA, 20 bp, Artificial Sequence
  Synthetic Necrotising Hepatopancreatitis/Hepatobacter penaei (NHP-B) 16SrRNA 2 primer BIP 2
actgctggcaactaagaacg

SEQ ID NO: 44 - DNA, 19 bp, Artificial Sequence
  Synthetic Necrotising Hepatopancreatitis/Hepatobacter penaei (NHP-B) 16SrRNA 2 primer F3
acgcgcaaaaccttaccag

SEQ ID NO: 45 - DNA, 20 bp, Artificial Sequence
  Synthetic Necrotising Hepatopancreatitis/Hepatobacter penaei (NHP-B) 16SrRNA 2 primer FIP 1
atgcagcacctgtacgggac

SEQ ID NO: 46 - DNA, 20 bp, Artificial Sequence
  Synthetic Necrotising Hepatopancreatitis/Hepatobacter penaei (NHP-B) 16SrRNA 2 primer FIP 2
tgacatagggacagaaggca

SEQ ID NO: 47 - DNA, 22 bp, Artificial Sequence
  Synthetic Necrotising Hepatopancreatitis/Hepatobacter penaei (NHP-B) 16SrRNA 2 primer LB
gtgagatgttgggttaagtccc

SEQ ID NO: 48 - DNA, 18 bp, Artificial Sequence
  Synthetic Necrotising Hepatopancreatitis/Hepatobacter penaei (NHP-B) 16SrRNA 2 primer LF
cgcaggcttcatctctga

SEQ ID NO: 49 - DNA, 18 bp, Artificial Sequence
  Synthetic Necrotising Hepatopancreatitis/Hepatobacter penaei (NHP-B) Flg E primer B3
cgggcgtttgcagttgat

SEQ ID NO: 50 - DNA, 20 bp, Artificial Sequence
  Synthetic Necrotising Hepatopancreatitis/Hepatobacter penaei (NHP-B) Flg E primer BIP 1
ggtccgctgacagcaccatt

SEQ ID NO: 51 - DNA, 17 bp, Artificial Sequence
  Synthetic Necrotising Hepatopancreatitis/Hepatobacter penaei (NHP-B) Flg E primer BIP 2
ggttgccagaaagagcc

SEQ ID NO: 52 - DNA, 19 bp, Artificial Sequence
  Synthetic Necrotising Hepatopancreatitis/Hepatobacter penaei (NHP-B) Flg E primer F3
catggaacaccctgtctcc

SEQ ID NO: 53 - DNA, 20 bp, Artificial Sequence
  Synthetic Necrotising Hepatopancreatitis/Hepatobacter penaei (NHP-B) Flg E primer FIP 1
ttcgctgttgagccagcctt

SEQ ID NO: 54 - DNA, 19 bp, Artificial Sequence
  Synthetic Necrotising Hepatopancreatitis/Hepatobacter penaei (NHP-B) Flg E primer FIP 2
caattcacgccccaaagca

SEQ ID NO: 55 - DNA, 19 bp, Artificial Sequence
  Synthetic Necrotising Hepatopancreatitis/Hepatobacter penaei (NHP-B) Flg E primer LB
ccctggatcccagcaccat

SEQ ID NO: 56 - DNA, 17 bp, Artificial Sequence
  Synthetic Necrotising Hepatopancreatitis/Hepatobacter penaei (NHP-B) Flg E primer LF
acaaacaccttgggcgg

SEQ ID NO: 57 - DNA, 19 bp, Artificial Sequence
  Synthetic Necrotising Hepatopancreatitis/Hepatobacter penaei (NHP-B) Gyr B primer B3
cgctggggaatgttgttgg

SEQ ID NO: 58 - DNA, 22 bp, Artificial Sequence
  Synthetic Necrotising Hepatopancreatitis/Hepatobacter penaei (NHP-B) Gyr B primer BIP 1
gccttgcatccaccttcatggt

SEQ ID NO: 59 - DNA, 20 bp, Artificial Sequence
  Synthetic Necrotising Hepatopancreatitis/Hepatobacter penaei (NHP-B) Gyr B primer BIP 2
tcatgataggtgtcggtcca

SEQ ID NO: 60 - DNA, 19 bp, Artificial Sequence
  Synthetic Necrotising Hepatopancreatitis/Hepatobacter penaei (NHP-B) Gyr B primer F3
ggcatcaccattgtgctga

SEQ ID NO: 61 - DNA, 20 bp, Artificial Sequence
  Synthetic Necrotising Hepatopancreatitis/Hepatobacter penaei (NHP-B) Gyr B primer FIP 1
acaaacgaagccacaccgcc

SEQ ID NO: 62 - DNA, 18 bp, Artificial Sequence
  Synthetic Necrotising Hepatopancreatitis/Hepatobacter penaei (NHP-B) Gyr B primer FIP 2
cggataagcgggatgagc

SEQ ID NO: 63 - DNA, 22 bp, Artificial Sequence
  Synthetic Necrotising Hepatopancreatitis/Hepatobacter penaei (NHP-B) Gyr B primer LB
gtgaaagacgatattgtgctgg

SEQ ID NO: 64 - DNA, 18 bp, Artificial Sequence
  Synthetic Necrotising Hepatopancreatitis/Hepatobacter penaei (NHP-B) Gyr B primer LF
tgcagggtgctttcctta

SEQ ID NO: 65 - DNA, 19 bp, Artificial Sequence
  Synthetic Infectious Hypodermal and Hematopoietic Necrosis Virus (IHHNV) Capsid protein primer B3
tctccatttggtcctttga

SEQ ID NO: 66 - DNA, 21 bp, Artificial Sequence
  Synthetic Infectious Hypodermal and Hematopoietic Necrosis Virus (IHHNV) Capsid protein primer BIP 1
atgggacaaaccatcccttgg

SEQ ID NO: 67 - DNA, 19 bp, Artificial Sequence
  Synthetic Infectious Hypodermal and Hematopoietic Necrosis Virus (IHHNV) Capsid protein primer BIP 2
tgaagatctgttgccagtc

SEQ ID NO: 68 - DNA, 21 bp, Artificial Sequence
  Synthetic Infectious Hypodermal and Hematopoietic Necrosis Virus (IHHNV) Capsid protein primer F3
cctatacaatccatggtgaca

SEQ ID NO: 69 - DNA, 21 bp, Artificial Sequence
  Synthetic Infectious Hypodermal and Hematopoietic Necrosis Virus (IHHNV) Capsid protein primer FIP 1
tgaccaccccattttgggatg

SEQ ID NO: 70 - DNA, 21 bp, Artificial Sequence
  Synthetic Infectious Hypodermal and Hematopoietic Necrosis Virus (IHHNV) Capsid protein primer FIP 2
cctatacaatccatggtgaca

SEQ ID NO: 71 - DNA, 21 bp, Artificial Sequence
  Synthetic Infectious Hypodermal and Hematopoietic Necrosis Virus (IHHNV) Capsid protein primer LB
aaacttagtctacccagcaga

SEQ ID NO: 72 - DNA, 20 bp, Artificial Sequence
  Synthetic Infectious Hypodermal and Hematopoietic Necrosis Virus (IHHNV) Capsid protein primer LF
ggtccggttgttggtatttc

SEQ ID NO: 73 - DNA, 18 bp, Artificial Sequence
  Synthetic Infectious Myonecrosis Virus (IMNV) ORF 1 primer B3
ttgtcgctggatgacatt

SEQ ID NO: 74 - DNA, 23 bp, Artificial Sequence
  Synthetic Infectious Myonecrosis Virus (IMNV) ORF 1 primer BIP 1
tgcatttgcaagaactggttcag

SEQ ID NO: 75 - DNA, 18 bp, Artificial Sequence
  Synthetic Infectious Myonecrosis Virus (IMNV) ORF 1 primer BIP 2
gtacttgcagtctacccg

SEQ ID NO: 76 - DNA, 23 bp, Artificial Sequence
  Synthetic Infectious Myonecrosis Virus (IMNV) ORF 1 primer F3
ttgaaatttctgattgtatgctg

SEQ ID NO: 77 - DNA, 23 bp, Artificial Sequence
  Synthetic Infectious Myonecrosis Virus (IMNV) ORF 1 primer FIP 1
acgctatgtatgataggtccagg

SEQ ID NO: 78 - DNA, 19 bp, Artificial Sequence
  Synthetic Infectious Myonecrosis Virus (IMNV) ORF 1 primer FIP 2
acctccagaccttacatcg

SEQ ID NO: 79 - DNA, 25 bp, Artificial Sequence
  Synthetic Infectious Myonecrosis Virus (IMNV) ORF 1 primer LB
ctgccacctttactttcaatactac

SEQ ID NO: 80 - DNA, 19 bp, Artificial Sequence
  Synthetic Infectious Myonecrosis Virus (IMNV) ORF 1 primer LF
attactctcaacgtccccg

SEQ ID NO: 81 - DNA, 18 bp, Artificial Sequence
  Synthetic Vibrio-spp.-causing Acute Hepatopancreatic Necrosis Disease/Early Mortality Syndrome Pir A primer B3
acagaaaccacgactagc

SEQ ID NO: 82 - DNA, 23 bp, Artificial Sequence
  Synthetic Vibrio-spp.-causing Acute Hepatopancreatic Necrosis Disease/Early Mortality Syndrome Pir A primer BIP 1
atgtggtacaacgtgatgaaact

SEQ ID NO: 83 - DNA, 24 bp, Artificial Sequence
  Synthetic Vibrio-spp.-causing Acute Hepatopancreatic Necrosis Disease/Early Mortality Syndrome Pir A primer BIP 2
gttaattacaacaatacgctgatg

SEQ ID NO: 84 - DNA, 20 bp, Artificial Sequence
  Synthetic Vibrio-spp.-causing Acute Hepatopancreatic Necrosis Disease/Early Mortality Syndrome Pir A primer BIP 2
agacattgagaatacgggac

SEQ ID NO: 85 - DNA, 22 bp, Artificial Sequence
  Synthetic Vibrio-spp.-causing Acute Hepatopancreatic Necrosis Disease/Early Mortality Syndrome Pir A primer FIP 1
tgatttagccactttccagccg

SEQ ID NO: 86 - DNA, 18 bp, Artificial Sequence
  Synthetic Vibrio-spp.-causing Acute Hepatopancreatic Necrosis Disease/Early Mortality Syndrome Pir A primer FIP 2
gtggggagcttaccattc

SEQ ID NO: 87 - DNA, 21 bp, Artificial Sequence
  Synthetic Vibrio-spp.-causing Acute Hepatopancreatic Necrosis Disease/Early Mortality Syndrome Pir A primer LB
taccatttacaacgccctgat

SEQ ID NO: 88 - DNA, 16 bp, Artificial Sequence
  Synthetic Vibrio-spp.-causing Acute Hepatopancreatic Necrosis Disease/Early Mortality Syndrome Pir A primer LF
aaatggcgcaccccat

SEQ ID NO: 89 - DNA, 19 bp, Artificial Sequence
  Synthetic Penaeus spp. 18SrRNA primer B3
gtggagctggaattaccgc

SEQ ID NO: 90 - DNA, 20 bp, Artificial Sequence
  Synthetic Penaeus spp. 18SrRNA primer BIP 1
gaacggggcctcgcaattgg

SEQ ID NO: 91 - DNA, 18 bp, Artificial Sequence
  Synthetic Penaeus spp. 18SrRNA primer BIP 2
gacttgccctccactcga

SEQ ID NO: 92 - DNA, 18 bp, Artificial Sequence
  Synthetic Penaeus spp. 18SrRNA primer F3
cggggaatcagggttcga

SEQ ID NO: 93 - DNA, 21 bp, Artificial Sequence
  Synthetic Penaeus spp. 18SrRNA primer FIP 1
ttcgtcactacctccccgtgc

SEQ ID NO: 94 - DNA, 20 bp, Artificial Sequence
  Synthetic Penaeus spp. 18SrRNA primer FIP 2
ctgagaaacggctaccacat

SEQ ID NO: 95 - DNA, 25 bp, Artificial Sequence
  Synthetic Penaeus spp. 18SrRNA primer LB
gtacactttaaatccttgtacgagg

SEQ ID NO: 96 - DNA, 18 bp, Artificial Sequence
  Synthetic Penaeus spp. 18SrRNA primer LF
gcctgctgccttccttag

SEQ ID NO: 97 - DNA, 615 bp, Artificial Sequence; identical to SEQ ID NO: 1
  Synthetic White Spot Syndrome Virus (WSSV) VP28 gene

SEQ ID NO: 98 - DNA, 520 bp, Infectious Hypodermal and Hematopoietic Necrosis Virus
atccccaacttgtgaccgtacaatcaacagcagcaaactatgaagacccaatccaacaatttggattcatggaacaaatgcgaaccggtgacagaaaagcctatacaagccatggtgacactagaaattggtatggcggagaaataccaacaaccggacccaccttcatcccaaaatggggtggtcaattaaaatgggacaaaccatcccttggaaacttagtctacccagcagaccaccatacaaacgactggcaacagatcttcatgagaatgtcaccaatcaaaggaccaaatggagacgaacttaaacttggctgcagagtacaagccgacttcttcctacacctagaagtacgactcccaccacaaggatgtgtcgcaagtttggggatgttacaatatcttcacgcaccatgtactggacagcttaacaaatgttatattatgcatactaactaaatatattcgatgtgcaatatatacccgattatatccagcttttaccaataaacatatat

SEQ ID NO: 99 - DNA, 614 bp, Hepatobacter penaei
cgttggaggttcgtccttcagtgtcgcagctaacgcgtgaagcattccgcctggggagtacggtcgcaagattaaaactcaaaggaattgacgggggcctgcacaagcggtggagcatgtggtttaattcgacgcaacgcgcaaaaccttaccagcttttgacatagggacagaaggcatcagagatgaagcctgcggttcggccgggtcccgtacaggtgctgcatggctgtcgtcagctcgtgtcgtgagatgttgggttaagtcccgcaacgagcgcaaccctcgttcttagttgccagcagttcggctggggactctaagaaaactgccggtgacaagccggaggaaggtggggatgatgtcaggtcctcatggcccttatgggctgggctacacacgtgctacaatggcgatcacaatgtgaagcaagggggtgacccggagccaatctctaaaaatcgtctcagttcggattgttctctgcaactcgagagcatgaagttggaatcgctagtaatcgcagatcagcatgctgcggtgaatacgttcacgggccttgtacacaccgcccgtcaagccatggaagttatcttttctttaaggcgatcagc

SEQ ID NO: 100 - DNA, 333 bp, Hepatobacter penaei
aggcaaacaaacaacccttggtaacacatggaacaccctgtctccccaattcacgccccaaagcatgccgcccaaggtgtttgtccaaggctggctcaacagcgaaggggtgccggtggggtccgctgacagcaccattgccctggatcccagcaccatggctctttctggcaaccaatatcaactgcaaacgcccgagcaagatgggcgtatggccgcccctttcaaaggcattagcatcagcgataccggggtgatttcttctgaattttttggtcaagaccccaaaggtgaatggcagttgtttttggtgaactttcccaacaacaacgc

SEQ ID NO: 101 - DNA, 20 bp, Artificial Sequence
  Synthetic Vibrio-spp.-causing Acute Hepatopancreatic Necrosis Disease/Early Mortality Syndrome Pir B F primer
tgatgaagtgatgggtgctc

SEQ ID NO: 102 - DNA, 20 bp, Artificial Sequence
  Synthetic Vibrio-spp.-causing Acute Hepatopancreatic Necrosis Disease/Early Mortality Syndrome Pir B R primer
tgtaagcgccgtttaactca

SEQ ID NO: 103 - DNA, 436 bp, Artificial Sequence
  Synthetic Vibrio parahaemolyticus causing-Acute hepatopancreatic necrosis disease (AHPND) Pir A
atgagtaacaatataaaacatgaaactgactattctcacgattggactgtcgaaccaaacggaggcgtcacagaagtagacagcaaacatacacctatcatcccggaagtcggtcgtagtgtagacattgagaatacgggacgtggggagcttaccattcaataccaatggggtgcgccatttatggctggcggctggaaagtggctaaatcacatgtggtacaacgtgatgaaacttaccatttacaacgccctgataatgcattctatcatcagcgtattgttgtaattaacaatggcgctagtcgtggtttctgtacaatctattaccactaagaaggtgctcacatgactaacgaatacgttgtaacaatgtcatctttgacggaatttaaccctaacaatgctcgtaaaagttatttatttgataactatg

SEQ ID NO: 104 - DNA, 400 bp, Artificial Sequence
  Synthetic Enterocytozoon hepatopenaei (EHP) SSU
tgtaaaaaccttgacgtgaagcaattggagggcaagttttggtgccagcagccgcggtaattccaactccaagagtgtctatggtggatgctgcagttaaagggtccgtagtcgtagatgcaattaaaaggtggtgttaaaagccattgagtttgttgagagtagcggaacggatagggagcatggtataggtgggcaaagaatgaaatctcaagaccccacctggaccaacggaggcgaaagcgatgctcttagacgtatctggggatcaaggacgaaggctagagtatcgaaagtgattagacaccgctgtagttctagcagtaaactatgccgacaatgctgggtgttgcgagagcgatgcttggtgtgggagaaatcttagttttcgggctctggg

SEQ ID NO: 105 - DNA, 851 bp, Artificial Sequence
  Synthetic penaeid shrimp 18SrRNA
tgccttatcagctctcgattgtaggttaaacgcctacaatggctatcacgggtaacggggaatcagggttcgattccggagagggagcctgagaaacggctaccacatctaaggaaggcagcaggcgcgcaaattacccactcccggcacggggaggtagtgacgaaaaatactgttgcgagccccgaacggggcctcgcaattggaatgagtacacttaaatccttgtacgaggatcgagtggagggcaagtctggtgccagccgccgcggtaattccagctccactagcgtatattaaagttgttgcggttgaaacgctcgtagtttgacttctgctccggaccggcggtccgccttagcggcggctactgccgggttccgagctgtgtccccgccggcgcacatggggtttttatgcccttaaccgggtgtccccttgtggccggcacgtttactttgaaaaaattagagtgctcagagcaggctggtttttgcttacagcccgaatggtcgtgcatggaatgatggaacaggacctcggttctattttgtcggtttttcggaacccgaggtaatgattaatagaagcagacgggggcattcgtactgcgacgctagaggtgaaaattcttagaccgtcgcatgacgacctactgcgaaagcatctgaggatgttttcattgatcaagaacgaaagttagaggttcgaaggcgatcagataccgccctagttctaaccttaaacgatgctgactagcgatccgccgcagttattcccatgacccggcgggtagcttccgggaaaccaaagtctttgagttccgggggaagtatggttgcaaagctgaa

SEQ ID NO: 106 - DNA, 614 bp, Artificial Sequence; identical to SEQ ID NO: 3
  Synthetic Hepatobacter penaei / Necrotising hepatopancreatitis (NHP) 16S 4RNA 2

SEQ ID NO: 107 - DNA, 990 bp, Artificial Sequence
  Synthetic Hepatobacter penaei / Necrotising hepatopancreatitis (NHP) gyrase B (GyrB)
gcaaacgccccggcatgtatatcggcaacaccgaagatggctcgggcctacatcacatggtgtatgaggtggtggacaacgcggtggatgaggcgctggccggtcattgcacacgcattcatgtgacgcttcaccccaatggctctgtcacggtaaaagatgacggccgcggcgtgcctgtggacattcatgcggaagaaggcgtgtccgcagcccaggtcatcatgacccacctacatgcagggggtaagtttgatgaaaacagctataaggtgtcaggcgggctgcacggcgtgggtgtgtcggtggtcaacgcgctgtccacgtggctggagatgcgcatttggcgggccggcaaagaacatgttatacgatttgaaaacggtgagcccgtggccccgttggcggtggtgggcgatgcggaaccggggcaaaccggcacacaagtcacctttttgccttcgcccaagacctttcagattaccaaattttcttttgacaccttagaaaatcgctttcgcgagcttgcgtttttgaaccccggcatcaccattgtgctgacggataagcgggatgagcctaaggaaagcaccctgcattatgagggcggtgtggcttcgtttgtggcctatctgaatcgcgcgcgcacggccctgcacacgccttgcatccaccttcatggtgtgaaagacgatattgtgctggatgtggcgatggaatggaccgacacctatcatgaaagcctcttgtgctttaccaacaacattccccagcgtgacggcggcacccacttgtcagggcttaaggcatctcttacccgggtggtgggtggctttgtgccaaaagcaatattcctaaaaaagacaaggtggccaccacgggcgaagatgtgcgcgaagggctgacgtgcgtgttgtcggtaaaggtgccggatccgaaattttcgtctcagaccaaagacaagttggtgtcttctgaggtgcgcagcgt

SEQ ID NO: 108 - DNA, 624 bp, Artificial Sequence
  Synthetic Vibrio parahaemolyticus causing-Acute hepatopancreatic necrosis disease (AHPND) Pir B
atgctgcggcaaaagatgattacattggtttagttactcattacttgattggacttgaagagaactttaagcgcgagctagacggtgatgaatggcttggttatgcgatattgcctctattagcaacaactgtaagtcttacaaattacttacatggcttgtggtctggattataaggatgaattcggtttcaccgattctgatgtgcataagctaacacgtaatattgataagctttatgatgatgtatcgtcttacattacagaactcgctgcgtgggctgataacgactcttacaataatgcaaaccaagataacgtgtatgatgaagtgatgggtgctcgtagttggtgtacggttcacggctttgaacatatgcttatttggcaaaaaatcaaagagttgaaaaaagttgatgtgtttgttcacagtaatttaatttcatattcacctgctgttggttttcctagtggtaatttcaactatattgctacaggtacggaagatgaaatacctcaaccattaaaaccaaatatgtttggggaacgtcgaaatcgtattgtaaaaattgaatcatggaacagtattgaaatacattattacaatcgcgtaggtcgacttaaa

SEQ ID NO: 109 - DNA, 18 bp, Artificial Sequence
  Synthetic NHP 16S rRNA primer for B3
cttagagtccccagccga

SEQ ID NO: 110 - DNA, 20 bp, Artificial Sequence
  Synthetic NHP 16S rRNA primer for BIP 1
ggctgtcgtcagctcgtgtc

SEQ ID NO: 111 - DNA, 20 bp, Artificial Sequence
  Synthetic NHP 16S rRNA primer for BIP 2
actgctggcaactaagaacg

SEQ ID NO: 112 - DNA, 19 bp, Artificial Sequence
  Synthetic NHP 16S rRNA primer for F3
acgcgcaaaaccttaccag

SEQ ID NO: 113 - DNA, 20 bp, Artificial Sequence
  Synthetic NHP 16S rRNA primer for FIP 1
atgcagcacctgtacgggac

SEQ ID NO: 114 - DNA, 20 bp, Artificial Sequence
  Synthetic NHP 16S rRNA primer for FIP 2
tgacatagggacagaaggca

SEQ ID NO: 115 - DNA, 22 bp, Artificial Sequence
  Synthetic NHP 16S rRNA primer for LB
gtgagatgttgggttaagtccc

SEQ ID NO: 116 - DNA, 18 bp, Artificial Sequence
  Synthetic NHP 16S rRNA primer for LF
cgcaggcttcatctctga

SEQ ID NO: 117 - DNA, 19 bp, Artificial Sequence
  Synthetic NHP GyrB primer B3
cgctggggaatgttgttgg

SEQ ID NO: 118 - DNA, 22 bp, Artificial Sequence
  Synthetic NHP GyrB primer BIP 1
gccttgcatccaccttcatggt

SEQ ID NO: 119 - DNA, 20 bp, Artificial Sequence
  Synthetic NHP GyrB primer BIP 2
tcatgataggtgtcggtcca

SEQ ID NO: 120 - DNA, 19 bp, Artificial Sequence
  Synthetic NHP GyrB primer F3
ggcatcaccattgtgctga

SEQ ID NO: 121 - DNA, 20 bp, Artificial Sequence
  Synthetic NHP GyrB primer FIP 1
acaaacgaagccacaccgcc

SEQ ID NO: 122 - DNA, 18 bp, Artificial Sequence
  Synthetic NHP GyrB primer FIP 2
cggataagcgggatgagc

SEQ ID NO: 123 - DNA, 22 bp, Artificial Sequence
  Synthetic NHP GyrB primer LB
gtgaaagacgatattgtgctgg

SEQ ID NO: 124 - DNA, 18 bp, Artificial Sequence
  Synthetic NHP GyrB primer LF
tgcagggtgctttcctta

SEQ ID NO: 125 - DNA, 24 bp, Artificial Sequence
  Synthetic PirB primer F3
gctaacacgtaatattgataagct

SEQ ID NO: 126 - DNA, 25 bp, Artificial Sequence
  Synthetic PirB primer B3
cacatcaacttttttcaactctttg

SEQ ID NO: 127 - DNA, 49 bp, Artificial Sequence
  Synthetic PirB primer FIP
tcatacacgttatcttggtttgcatgtatcgtcttacattacagaactc

SEQ ID NO: 128 - DNA, 42 bp, Artificial Sequence
  Synthetic PirB primer BIP
aagtgatgggtgctcgtagttgccaaataagcatatgttcaa

SEQ ID NO: 129 - DNA, 21 bp, Artificial Sequence
  Synthetic PirB primer LF
tgtaagagtcgttatcagccc

SEQ ID NO: 130 - DNA, 19 bp, Artificial Sequence
  Synthetic PirB primer LB
tggtgtacggttcacggct

SEQ ID NO: 131 - DNA, 24 bp, Artificial Sequence
  Synthetic PirB primer F2
gtatcgtcttacattacagaactc

SEQ ID NO: 132 - DNA, 25 bp, Artificial Sequence
  Synthetic PirB primer F1c
tcatacacgttatcttggtttgcat

SEQ ID NO: 133 - DNA, 22 bp, Artificial Sequence
  Synthetic PirB primer B2
tgccaaataagcatatgttcaa

SEQ ID NO: 134 - DNA, 20 bp, Artificial Sequence
  Synthetic PirB primer B1c
aagtgatgggtgctcgtagt

SEQ ID NO: 135 - DNA, 18 bp, Artificial Sequence
  Synthetic EHP beta tubulin primer F3
ctctgtgttcagaagcgt

SEQ ID NO: 136 - DNA, 21 bp, Artificial Sequence
  Synthetic EHP beta tubulin primer B3
caaattcatctgctcgtctac

SEQ ID NO: 137 - DNA, 48 bp, Artificial Sequence
  Synthetic EHP beta tubulin primer FIP
catcatgtttttgctgtcaaaaagccttcaagctacagaaatcttacg

SEQ ID NO: 138 - DNA, 43 bp, Artificial Sequence
  Synthetic EHP beta tubulin primer BIP
actgcgtgtgacccaagaaattctttcattgacattttccctc

SEQ ID NO: 139 - DNA, 20 bp, Artificial Sequence
  Synthetic EHP beta tubulin primer LF
tgtgctgtcaaatctgcaac

SEQ ID NO: 140 - DNA, 25 bp, Artificial Sequence
  Synthetic EHP beta tubulin primer LB
ggaaggtatttgacagcagctgttt

SEQ ID NO: 141 - DNA, 23 bp, Artificial Sequence
  Synthetic EHP beta tubulin primer F2
cttcaagctacagaaatcttacg

SEQ ID NO: 142 - DNA, 25 bp, Artificial Sequence
  Synthetic EHP beta tubulin primer F1c
catcatgtttttgctgtcaaaaagc

SEQ ID NO: 143 - DNA, 23 bp, Artificial Sequence
  Synthetic EHP beta tubulin primer B2
ttctttcattgacattttccctc

SEQ ID NO: 144 - DNA, 20 bp, Artificial Sequence
  Synthetic EHP beta tubulin primer B1c
actgcgtgtgacccaagaaa

SEQ ID NO: 145 - DNA, 514 bp, Artificial Sequence
  Synthetic EHP beta tubulin
caaatttaaacagtcttgtgtccaaagttatgagtggaattaccacttgttttagatttccaggtcaacttaactcagatctgagaaaattggcggttaacatgattccatttccacgactgcactttttctgtgtaggatatgcgcctctgtgttcagaagcgtcttcaagctacagaaatcttacggttgcagatttgacagcacagctttttgacagcaaaaacatgatgactgcgtgtgacccaagaaaaggaaggtatttgacagcagctgtttattttagagggaaatgtcaatgaaagaagtagacgagcagatgaatttggcacaaacaagaactagtgattcttttgtcgagtggattccaaacaacgtgcaaacggctgtttgtaacgtagcaccaaatgacgtagaaatgagcgctacattcataggcaacacaacttcaattcaagagattttcatgcgtcttggtgagcagttttcaagcatgtttaaacgaaaggcattt

SEQ ID NO: 146 - DNA, 18 bp, Artificial Sequence
  Synthetic IMNV RdRP primer F3
attcaagtgggcacatgc

SEQ ID NO: 147 - DNA, 20 bp, Artificial Sequence
  Synthetic IMNV RdRP primer B3
tcgatcaagtcttgagatgt

SEQ ID NO: 148 - DNA, 42 bp, Artificial Sequence
  Synthetic IMNV RdRP primer FIP
gcacacaactttgtgattgcgagagacaacgtgtatatgaca

SEQ ID NO: 149 - DNA, 43 bp, Artificial Sequence
  Synthetic IMNV RdRP primer BIP
cgaaagtcgggttttatgaatgcgtgcttgatctataacagcg

SEQ ID NO: 150 - DNA, 17 bp, Artificial Sequence
  Synthetic IMNV RdRP primer LF
catccctggacgactgc

SEQ ID NO: 151 - DNA, 19 bp, Artificial Sequence
  Synthetic IMNV RdRP primer LB
cacccaagactggactcag

SEQ ID NO: 152 - DNA, 21 bp, Artificial Sequence
  Synthetic IMNV RdRP primer F2
agagacaacgtgtatatgaca

SEQ ID NO: 153 - DNA, 21 bp, Artificial Sequence
  Synthetic IMNV RdRP primer F1c
gcacacaactttgtgattgcg

SEQ ID NO: 154 - DNA, 20 bp, Artificial Sequence
  Synthetic IMNV RdRP primer B2
gtgcttgatctataacagcg

SEQ ID NO: 155 - DNA, 23 bp, Artificial Sequence
  Synthetic IMNV RdRP primer B1c
cgaaagtcgggttttatgaatgc

SEQ ID NO: 156 - DNA, 621 bp, Artificial Sequence
  Synthetic IMNV RdRP
aattatgatggaaccgcaacaaacgcaaacgtcgtaccaacaggatcatacattaagcagagagcaatgcctattaatgcaaatgcggtacgaccaactgaaacactcgacgctgctaaccatacaaaaccttttgctattgaaggaggaagactcgtatatttgggtggaacaattgcaaatacaaccaatgtggtaaacgcgatgcagaggaaacaaaggctttcaaaaccggcattcaagtgggcacatgctcagagacaaacgtgtatatgacagcagtcgtccagggatggacgcaatcacaaagttgtgtgcacgaaagtcgggttttatgaatgcccgttccacagcaatgatggcacccaagactggactcagcgctgttatagatcaagcaccaaatacatctcaagacttgatcgaacagccgagtcagcaagaggttatggatatgcaagcgacagcaacagtataaatcagatatatcaaattgcattgcatagaaaggcaaaacttataacagcaaagaaatggcaagaattaacaaaaggtatttataatgcatctaccctgacaccgaagatagttgaccaaattataaaggatgaaggaagtggg

SEQ ID NO: 157 - DNA, 19 bp, Artificial Sequence
  Synthetic V. parahaemolyticus Tox R primer F3
gttccaaaacgaggctatc

SEQ ID NO: 158 - DNA, 20 bp, Artificial Sequence
  Synthetic V. parahaemolyticus Tox R primer B3
ctttttcaggtactactggc

SEQ ID NO: 159 - DNA, 45 bp, Artificial Sequence
  Synthetic V. parahaemolyticus Tox R primer FIP
gctcttcaacttcaattgagcttgacatttgtactgttgaacgcc

SEQ ID NO: 160 - DNA, 43 bp, Artificial Sequence
  Synthetic V. parahaemolyticus Tox R primer LF
caatgacgcctctactaatgaggtgcttctggttcaacgattg

SEQ ID NO: 161 - DNA, 19 bp, Artificial Sequence
  Synthetic V. parahaemolyticus Tox R primer LF
tctgaagaaagcgggctta

SEQ ID NO: 162 - DNA, 18 bp, Artificial Sequence
  Synthetic V. parahaemolyticus Tox R primer LB
gagccgtctttagcgacg

SEQ ID NO: 163 - DNA, 20 bp, Artificial Sequence
  Synthetic V. parahaemolyticus Tox R primer F2
catttgtactgttgaacgcc

SEQ ID NO: 164 - DNA, 25 bp, Artificial Sequence
  Synthetic V. parahaemolyticus Tox R primer F1c
gctcttcaacttcaattgagcttga

SEQ ID NO: 165 - DNA, 19 bp, Artificial Sequence
  Synthetic V. parahaemolyticus Tox R primer B2
gcttctggttcaacgattg

SEQ ID NO: 166 - DNA, 24 bp, Artificial Sequence
  Synthetic V. parahaemolyticus Tox R primer B1c
caatgacgcctctactaatgaggt

SEQ ID NO: 167 - DNA, 954 bp, Artificial Sequence
  Synthetic V. parahaemolyticus Tox R
tcatccatctaagaagaactaaatgactaacatcggcaccaaatttctacttgctcaaaggtttacctttgatccaaatagtaattcgctcgctgaccaacaaagcggcaacgaagttgtacgattaggaagcaacgaaagccgtatactcctgatgttggcggagagaccaaacgaagttttaacccgtaacgagcttcacgagtttgtttggcgtgagcaaggttttgaggtggatgactcaagcctgactcaagcgatttctactctgcgtaagatgttgaaggattcaaccaaatctccagagtttgttaaaaccgttccaaaacgaggctatcaactcatttgtactgttgaacgcctaagcccgctttcttcagactcaagctcaattgaagttgaagagccagcttctgataacaatgacgcctctactaatgaggtagaaaacgatcgtagagccgtctttagcgacgacttctgacgcaatcgttgaaccagaagcgccagtagtacctgaaaaagcacctgtggcttctgctgtgaatccttggattccacgcgttattttatttttggcactattactaccgatttgcgtactgctgtttacaaacccagcggaatctcagttccgtcagattggtgagtatcagaacgtaccagtgatgacacctgtaaatcacccgcaaatcaacaactggttgccttctattgagcagtgcattgaacgctacgttaagcaccatgcagaagactgttaccagtggaagtgattgccactggcggacaaaataaccagctgattttgaactacattcatgacagcaaccactcgtatgagaacgtgacattgcgtattttcgcaggtcaaaacgatccaacagacatctgcaaataaaggaggccagcatgaagattaaagtagcatctgcggttttggccgtatctatc

SEQ ID NO: 168 - DNA, 19 bp, Artificial Sequence
  Synthetic Tilapia lake virus (TiLV) segment 3 primer F3
tcgtattgagtgggtggca

SEQ ID NO: 169 - DNA, 18 bp, Artificial Sequence
  Synthetic Tilapia lake virus (TiLV) segment 3 primer B3
ccctccattgcggaactc

SEQ ID NO: 170 - DNA, 40 bp, Artificial Sequence
  Synthetic Tilapia lake virus (TiLV) segment 3 primer FIP
gcgaggaactttgagcactcgaacccagacttgcggacat

SEQ ID NO: 171 - DNA, 39 bp, Artificial Sequence
  Synthetic Tilapia lake virus (TiLV) segment 3 primer BIP
ctgcggtgaggagtgtggtcacgtgcgtactcgttcagt

SEQ ID NO: 172 - DNA, 21 bp, Artificial Sequence
  Synthetic Tilapia lake virus (TiLV) segment 3 primer LF
tttagctcagctgtttccttg

SEQ ID NO: 173 - DNA, 20 bp, Artificial Sequence
  Synthetic Tilapia lake virus (TiLV) segment 3 primer LB
ttgaccaagaggcaagagaa

SEQ ID NO: 174 - DNA, 18 bp, Artificial Sequence
  Synthetic Tilapia lake virus (TiLV) segment 3 primer F2
acccagacttgcggacat

SEQ ID NO: 175 - DNA, 22 bp, Artificial Sequence
  Synthetic Tilapia lake virus (TiLV) segment 3 primer F1c
gcgaggaactttgagcactcga

SEQ ID NO: 176 - DNA, 19 bp, Artificial Sequence
  Synthetic Tilapia lake virus (TiLV) segment 3 primer B2
acgtgcgtactcgttcagt

SEQ ID NO: 177 - DNA, 20 bp, Artificial Sequence
  Synthetic Tilapia lake virus (TiLV) segment 3 primer B1c
ctgcggtgaggagtgtggtc

SEQ ID NO: 178 - DNA, 1367 bp, Artificial Sequence
  Synthetic Tilapia lake virus (TiLV) segment 3
gcaaatttttcccataatcctctattagaacgtcgtaacctttagcgaaggcgtcgaaagcgatcatctcgcaaatgggtgtactgtcatccgcaatcttactgcacaaagtgaataataaagtgagcttaagggtattgtaccccttatctagaagccagctggtagcctttcctgaactcgtccttacagacgctaagtgctagccggtgcctattaaaatgttgagccactgcttggctaacccttgtagagtcgaggcattccagaagtaagatgacgtcccatcttgtctcaagaccactagctctgtccagatcacccttcctacttatgggaggtagttccaacatatccagcttgtaaatttctcgggtactcacaaagtcttgctcccctatattagccgattgcttgggatctaggtgcatcacatgcacagctgccctgtacccgtcaaactcaaaatcgtgttcacarccaggtttacttagacctacaactaagtggtgagtggaggcggttggtctccttttactgtgctttccagagtcgcgcatgaccggtacagctagtatgctggtattatcgctatgcagtactttccctgcctgagttgtgcttctagcaatcaacatcaaaagctcacgagcaagtggrgcactagcccgtagaggtaatatcttctgtgtagcaggcttrtgagaagcaactgtatacctttgtatccaccctccattgcggaactcaaattctctatcacgtgcgtactgttcagtataagttctcttgcctcttggtcaagaccacactcctcaccgcaggcgaggaactttgagcactcgaagaatccatattgcctctttagctcagtctttccttggatatgtccgcaagtctgggtggtgccacccactaatacgaggcttcgggccactctttggatgtggtagttccaatagccgttcccttagctcagcatcgtaggatgccttgtgcccaactttaacaaccccgtcgatctccaaataatccttgaattgtaccttacaaatctgctggtactcttgctcagagcaaacgtatgtagcatcattgttcaaaatgcaacgaactgttgcctttggaaatttccgcggctggccttccagctcaaaggcaatccacttattcttcaaacagtcataacagtcaccctctacggggactcctggacgtctctctactgtactgtaagaacttaggaaccttcggctcccttcgctataagtgaaatcgtcacagaaaaccccagttagctgtgcaaaccgcgagtccatttcaaaagaaagttaacggtctattaaggattaaggggtaatatttgg

SEQ ID NO: 179 - DNA, 19 bp, Artificial Sequence
  Synthetic Tilapia lake virus (TiLV) segment 8 primer F3
gccaagggaagctctacga

SEQ ID NO: 180 - DNA, 19 bp, Artificial Sequence
  Synthetic Tilapia lake virus (TiLV) segment 8 primer B3
actcgcctcgagattcgtc

SEQ ID NO: 181 - DNA, 41 bp, Artificial Sequence
  Synthetic Tilapia lake virus (TiLV) segment 8 primer FIP
gcaaatccggacctgcattgacctcaacggcatgacagtga

SEQ ID NO: 182 - DNA, 41 bp, Artificial Sequence
  Synthetic Tilapia lake virus (TiLV) segment 8 primer BIP
attggcgaaggcctctcagaagcctctatccgctttcgggt

SEQ ID NO: 183 - DNA, 21 bp, Artificial Sequence
  Synthetic Tilapia lake virus (TiLV) segment 8 primer LF
actacagtgttgactgtgtct

SEQ ID NO: 184 - DNA, 19 bp, Artificial Sequence
  Synthetic Tilapia lake virus (TiLV) segment 8 primer LB
cctgcttgttaagcacgcc

SEQ ID NO: 185 - DNA, 19 bp, Artificial Sequence
  Synthetic Tilapia lake virus (TiLV) segment 8 primer F2
ctcaacggcatgacagtga

SEQ ID NO: 186 - DNA, 22 bp, Artificial Sequence
  Synthetic Tilapia lake virus (TiLV) segment 8 primer F1c
gcaaatccggacctgcattgac

SEQ ID NO: 187 - DNA, 19 bp, Artificial Sequence
  Synthetic Tilapia lake virus (TiLV) segment 8 primer B2
cctctatccgctttcgggt

SEQ ID NO: 188 - DNA, 22 bp, Artificial Sequence
  Synthetic Tilapia lake virus (TiLV) segment 8 primer B1c
attggcgaaggcctctcagaag

SEQ ID NO: 189 - DNA, 525 bp, Artificial Sequence
  Synthetic Tilapia lake virus (TiLV) segment 8
atggctcaaatcccaacactaagagagggccaagggaagctctacgatttcacgctcaacggcatgacagtgactagagacacagtcaacactgtagttgctctggagtttcttgtcaatgcaggtccggatttgctttccctaacaattggcgaaggcctctcagaagaaacaaagtttaaacacctgcttgttaagcacgccggcatgacccgaaagcggatagaggaaaggctggggacgaatctcgaggcgagtcagtgtgaagtcgacgcaattataataacaaaccgcaagggtcaaagatttgaattcaatcggaagcagtacctggatattgccaaacaagctatgaagcttaagctccctgggattaactgtgtcgacatacccactgcgctcgcttttctcgaggaggtcctggcaactgctttgaaggacactgaaggttcacaagatgacaggatggcccttaaggcagacacttctgctgctatcaatcatttccgtgaaatgcttaaataa

SEQ ID NO: 190 - DNA, 360 bp, Artificial Sequence
  Synthetic Vibrio spp.-causing Acute Hepatopancreatic Necrosis Disease/Early Mortality Syndrome Pir A
atgagtaacaatataaaacatgaaactgactattctcacgattggactgtcgaaccaaacggaggcgtcacagaagtagacagcaaacatacacctatcatcccggaagtcggtcgtagtgtagacattgagaatacgggacgtggggagcttaccattcaataccaatggggtgcgccatttatggctggcggctggaaagtggctaaatcacatgtggtacaacgtgatgaaacttaccatttacaacgccctgataatgcattctatcatcagcgtattgttgtaattaacaatggcgctagtcgtggtttctgtacaatctattaccactaagaaggtgctcacatgactaacgaa

SEQ ID NO: 191 - DNA, 1668 bp, Artificial Sequence
  Synthetic Vibrio-spp.-causing Acute Hepatopancreatic Necrosis Disease/Early Mortality Syndrome Pir B
atgagtaacaatataaaacatgaaactgactattctcacgattggactgtcgaaccaaacggaggcgtcacagaagtagacagcaaacatacacctatcatcccggaagtcggtcgtagtgtagacattgagaatacgggacgtggggagcttaccattcaataccaatggggtgcgccatttatggctggcggctggaaagtggctaaatcacatgtggtacaacgtgatgaaacttaccatttacaacgccctgataatgcattctatcatcagcgtattgttgtaattaacaatggcgctagtcgtggtttctgtacaatctattaccactaagaaggtgctcacatgactaacgaatacgttgtaacaatgtcatctttgacggaatttaaccctaacaatgctcgtaaaagttatttatttgataactatgaagttgatcctaactatgctttcaaagcaatggtttcatttggtctttcaaatattccttacgcgggtggttttttatcaacgttatggaatatcttttggccaaatacgccaaatgagccagatattgaaaacatttgggaacaattacgtgacagaatccaagatttagtagatgaatcgattatagatgccatcaatggaatattggatagcaaaatcaaagagacacgcgataaaattcaagacattaatgagactatcgaaaacttcggttatgctgcggcaaaagatgattacattggtttagttactcattacttgattggacttgaagagaactttaagcgcgagctagacggtgatgaatggcttggttatgcgatattgcctctattagcaacaactgtaagtcttcaaattacttacatggcttgtggtctggattataaggatgaattcggtttcaccgattctgatgtgcataagctaacacgtaatattgataagctttatgatgatgtatcgtcttacattacagaactcgctgcgtgggctgataacgactcttacaataatgcaaaccaagataacgtgtatgatgaagtgatgggtgctcgtagttggtgtacggttcacggctttgaacatatgcttatttggcaaaaaatcaaagagttgaaaaaagttgatgtgtttgttcacagtaatttaatttcatattcacctgctgttggttttcctagtggtaatttcaactatattgctacaggtacggaagatgaaatacctcaaccattaaaaccaaatatgtttggggaacgtcgaaatcgtattgtaaaaattgaatcatggaacagtattgaaatacattattacaatcgcgtaggtcgacttaaactaacttatgaaaatggggaagtggtagaactaggcaaggctcataaatatgacgagcattaccaatctattgagttaaacggcgcttacattaaatatgttgatgttattgccaatggacctgaagcaattgatcgaatcgtatttcatttttcagatgatcgaacatttgttgttggtgaaaactcaggcaagccaagtgtgcgtttgcaactggaaggtcattttatttgtggcatgcttgcggatcaagaaggttctgacaaagttgccgcgtttagcgtggcttatgaattgtttcatcccgatgaatttggtacagaaaagtagtaa

SEQ ID NO: 192 - DNA, 870 bp, Artificial Sequence
  Synthetic Enterocytozoon hepatopenaei (EHP) Beta tubulin
cactacacagagggtgcggaactatgcgaacaggtgttggaatgcataagaaaggaagcagaaaaaacagactgtcttcagggattccaacttacacactccttgggtggtggaacaggttcgggaatgggaacattgctggtatcaaaaataaaggaggaatttccagataggatgcttgcaacattttctgtagttccaagtccaaaagtttctgacactgtggttgaaccatacaatgcaacactctcttttcaccagctggttgaaaatgcaaatcaaacattttgcatcgataatgacgcgctttatgaaatatgcacaaaaacactcaaattaaaaaatccatcttatgacaatttaaacagtcttgtgtccaaagttatgagtggaattaccacttgttttagatttccaggtcaacttaactcagatctgagaaaattggcggttaacatgattccatttccacgactgcactttttctgtgtaggatatgcgcctctgtgttcagaagcgtcttcaagctacagaaatcttacggttgcagatttgacagcacagctttttgacagcaaaaacatgatgactgcgtgtgacccaagaaaaggaaggtatttgacagcagctgtttattttagagggaaaatgtcaatgaaagaagtagacgagcagatgaatttggcacaaacaagaactagtgattcttttgtcgagtggattccaaacaacgtgcaaacggctgtttgtaacgtagcaccaaatgacgtagaaatgagcgctacattcataggcaacacaacttcaattcaagagattttcatgcgtcttggtgagcagttttcaagcatgtttaaacgaaaggcattt

SEQ ID NO: 193 - DNA, 1501 bp, Artificial Sequence
  Synthetic Infectious Myonecrosis virus (IMNV) RdRp
gacgtatttcaacaaaggcgtgggacacagcatatgatagtaacatccaagcatatcaggacatgcattatcaaatgttttcgtggagttcaatgcaaggtagtattgcgccagcaatggtggacgaaattcttcataacctttgtggccaaatgtttggcttcagcttaccattgagacaagtcttatttaacgcattgccaatcactttttcatcgtttggaagttggatgttgcctagagtttctgatggtttccaaactgtaaggtattatgatgcaggtccaccagccattaatgcaaaacgtgatggggaagtaccagtaagtatgattgacgcatggacctataaatttacagaaaaattgccaaaaagttttttgccatggccaatgccagaaggaaaggacagtacaatgggatatgatccggaaaaagaaccagcactaattgataattcaaatgagacaggcaatgtattcagaccattcatggcaagaaatggcaacaattccaattatttaccaaccaactacacaattgacgtatcacagaatggtcatgatgagagttgtattaatgttgacctttttaacaatgttgcaggagtaacactaacaaattatgatggaaccgcaacaaacgcaaacgtcgtaccaacaggatcatacattaagcagagagcaatgcctattaatgcaaatgcggtacgaccaactgaaacactcgacgctgctaaccatacaaaaccttttgctattgaaggaggaagactcgtatatttgggtggaacaattgcaaatacaaccaatgtggtaaacgcgatgcagaggaaacaaaggctttcaaaaccggcattcaagtgggcacatgctcagagacaacgtgtatatgacagcagtcgtccagggatggacgcaatcacaaagttgtgtgcacgaaagtcgggttttatgaatgcccgttccacagcaatgatggcacccaagactggactcagcgctgttatagatcaagcaccaaatacatctcaagacttgatcgaacagccgagtcagcaagaggttatggatatgcaagcgacagcaacagtataaatcagatatatcaaattgcattgcatagaaaggcaaaacttataacagcaaagaaatggcaagaattaacaaaaggtatttataatgcatctaccctgacaccgaagatagttgaccaaattataaaggatgaaggaagtgggaccgataagacaaaatatgtaaatgttcctaaaataattactgacaaagaattacaaacattctatgtaccaagaagcaacgcagacctagttataagaagaatacgtttaatcgacctttggcgtaacctaaaaccagatcaaatggacgagattcgtaattacactcatctagattatatctttgtacaaaacatttgtatctatatgttagtatttggaatagacacagttaaacactttagacaaataggtctattc

SEQ ID NO: 194 - DNA, 1364 bp, Artificial Sequence
  Synthetic Vibrio parahaemolyticus ToxR
tcatccatctaagaagaactaaatgactaacatcggcaccaaatttctacttgctcaaaggtttacctttgatccaaatagtaattcgctcgctgaccaacaaagcggcaacgaagttgtacgattaggaagcaacgaaagccgtatactcctgatgttggcggagagaccaaacgaagttttaacccgtaacgagcttcacgagtttgtttggcgtgagcaaggttttgaggtggatgactcaagcctgactcaagcgatttctactctgcgtaagatgttgaaggattcaaccaaatctccagagtttgttaaaaccgttccaaaacgaggctatcaactcatttgtactgttgaacgcctaagcccgctttcttcagactcaagctcaattgaagttgaagagccagcttctgataacaatgacgcctctactaatgaggtagaaacgatcgtagagccgtctttagcgacgacttctgacgcaatcgttgaaccagaagcgccagtagtacctgaaaaagcacctgtggcttctgctgtgaatccttggattccacgcgttattttatttttggcactattactaccgatttgcgtactgctgtttacaaacccagcggaatctcagttccgtcagattggtgagtatcagaacgtaccagtgatgacacctgtaaatcacccgcaaatcaacaactggttgccttctattgagcagtgcattgaacgctacgttaagcaccatgcagaagactcgttaccagtggaagtgattgccactggcggacaaaataaccagctgattttgaactacattcatgacagcaaccactcgtatgagaacgtgacattgcgtattttcgcaggtcaaaacgatccaacagacatctgcaaataaaggaggccagcatgaagattaaagtagcatctgcggttttggccgtatctatccttttcagtggttggttgtactggggcagtgaccttaaagttgagcaagtgcttacatcaaatgaatggcagtcaaccatggtgactgtaattactgataacttgccagacgataccgtaggcccgttacgtcgagtgaatgtggagtcgaacgttaaatacctgccgaatggcgattacattcgcgtggcaaacatcaaactgttcgcacaaggctcgacggctgaatcgacaattaatatttcagagaaaggtcgctgggaagtgagtgataactatctgcttgtttctccttctgagttcaaagatatttcttcttctcaatccaaggatttttctgaagcgcaactacgtttaattactcaaatctttaagctagatgcagaacaaagccgccgaattgacgtggt

SEQ ID NO: 195 - DNA, 1371 bp, Artificial Sequence
  Synthetic Tilapia lake virus (TiLV) Segment 3
gcaaatttttcccataatcctctattagaacgtcgtaacctttagcgaaggcgtcgaaagcgatcatctcgcaaatgggtgtactgtcatccgcaatcttactgcacaaagtgaataataaagtgagcttaagggtattgtaccccttatctcagaagccagctggtagcctttcctgaactcgtccttacagacgctaagtgctagccggtgcctattaaaatgttgagccactgcttggctaacccttgtagagtcgaggcattccagaagtaagatgacgtcccatcttgtctcaagaccactagctctgtccagatcacccttcctacttatgggaggtagttccaacatatccagcttgtaaatttctcgggtactcacaaagtcttgctcccctatattagccgattgcttgggatctaggtgcatcacatgcacagctgccctgtacccgtcaaactcaaaatcgtgttcacarccaggtttacttagacctacaactaagtggtgagtggaggcggttggtctccttttactgtgctttccagagtcgcgcatgaccggtacagctagtatgctggtattatcgctatgcagtactttccctgcctgagttgtgcttctagcaatcaacatcaaaagctcacgagcaagtggrgcactagccggtagaggtaatatcttctgtgtagcaggcttrtgagaagcaactgtatacctttgtatccaccctccattgcggaactcaaattctctatcacgtgcgtactcgttcagtataagttctcttgcctcttggtcaagaccacactcctcaccgcaggcgaggaactttgagcactcgaagaatccatattgcctctttagctcagctgtttccttggatatgtccgcaagtctgggtggtgccacccactcaatacgaggcttcgggccactctttggatgtggtagttccaatagccgttcccttagctcagcatcgtaggatgccttgtgcccaactttaacaaccccgtcgatctccaaataatccttgaattgtaccttacaaatctgctggtactcttgctcagagcaaacgtatgtagcatcattgttcaaaatgcaacgaactgttgcctttggaaatttccgcggctggccttccagctcaaaggcaatccacttattcttcaaacagtcataacagtcaccctctacggggactcctggacgtctctctactgtactgtaagaacttaggaaccttcggctcccttcgctataagtgaaatcgtcacagaaaaccccagttagctgtgcaaaccgcgagtccatttcaaaagaaagttaacggtctattaaggattaaggggtaatatttgg

SEQ ID NO: 196 - DNA, 341 bp, Artificial Sequence
  Synthetic Infectious Myconecrosis virus (IMNV) capsid protein (CP) gene
gattttgtaaatgggacccaacctacattgaaatttctgattgtatgctgccacctccagaccttacatcgtgcggggacgttgagagtaatcctggacctatcatacatagcgttgcatttgcaagaactggttcagtatggacacctgccacctttactttcaatactacatcatccccgggtagactgcaagtacaaatgtcatccagcgacaatagatatgggttcaattctgttttacttgcagcaggtggtacaacatggggcacagcttatttttcacaacgctggaacagtgactttggattacagccagatctaactataactgtaacacca

(19) United States
(12) Patent Application Publication
(10) Pub. No.: US 2024/0352543 A1
(43) Pub. Date: Oct. 24, 2024

(54) ISOTHERMAL AMPLIFICATION-BASED DETECTION OF SHRIMP PATHOGENS

(71) Applicant: ARIZONA BOARD OF REGENTS ON BEHALF OF THE UNIVERSITY OF ARIZONA, Tucson, AZ (US)

(72) Inventors: Luis Fernando Aranguren Caro, Tucson, AZ (US); Arun K Dhar, Tucson, AZ (US); Hung Nam Mai, Tucson, AZ (US)

(21) Appl. No.: 18/285,876

(22) PCT Filed: Apr. 8, 2022
(86) PCT No.: PCT/US2022/024122

§ 371 Date: Oct. 6, 2023

Related U.S. Application Data
(60) Provisional application No. 63/173,254, filed on Apr. 9, 2021.

(51) Int. Cl. C12Q 1/70; C12Q 1/6844; C12Q 1/689

(52) U.S. Cl. CPC C12Q 1/701; C12Q 1/6844; C12Q 1/689; C12Q 2600/166

(57) ABSTRACT

Described herein are assays capable of detecting one or more shrimp or fish pathogens. Also described herein are kits configured for detecting one or more shrimp or fish pathogens. In some embodiments, the assays and kits are configured as a point of care test for one or more shrimp or fish pathogens.

### CROSS-REFERENCE TO RELATED APPLICATIONS

This application claims the benefit of and priority to co-pending U.S. Provisional Patent Application No. 63/173,254, filed on Apr. 9, 2021, entitled “ISOTHERMAL AMPLIFICATION-BASED DETECTION OF SHRIMP PATHOGENS,” the contents of which is incorporated by reference herein in its entirety.

### SEQUENCE LISTING

This application contains a sequence listing filed in electronic form as an ASCII.txt file entitled UAZ-0135WP_ST25.txt, created on Apr. 8, 2022 and having a size of 74,590 bytes (78 KB on disk). The content of the sequence listing is incorporated herein in its entirety.

### TECHNICAL FIELD

The subject matter disclosed herein is generally directed to detection of crustacean, particularly shrimp, pathogens.

### BACKGROUND

Aquaculture farms, especially shrimp farms, is a rapidly growing market as they provide an abundant, cheap source of protein. The global shrimp market is currently valued at about $40 billion and is expected to increase to $68 billion by 2028. Of key concern to this market is aquaculture disease. Pathogens, such as bacterial, viral, and fungal pathogens, are a threat to the growth and expansion of the industry. An important aspect of pathogen control and treatment is testing and surveillance. As such there is urgent need for pathogen testing methods for aquaculture pathogens, particularly crustacean pathogens, and more particularly shrimp pathogens.

Citation or identification of any document in this application is not an admission that such a document is available as prior art to the present invention.

### SUMMARY

Described in certain example embodiments herein are kits configured to detect one or more shrimp or fish pathogens using loop-mediate isothermal amplification (LAMP), the kit comprising one or more primer sets configured to specifically amplify one or more shrimp or fish pathogen-specific target polynucleotides via LAMP; and one or more amplification reagents.

In certain example embodiments, the kit further comprises one or more signal reagents.

In certain example embodiments, at least one of the one or more signal reagents is a pH sensitive reagent that is capable of changing color from a first visible color to a second visible color in response to amplification of a target polynucleotide.

In certain example embodiments, the first visible color and the second visible color are different colors.

In certain example embodiments, the pH sensitive indicator changes color when the LAMP amplification moves from a neutral or basic pH to an acid pH as the reaction proceeds.

In certain example embodiments, the pH sensitive indicator is phenol red, cresol red, phenolphthalein, methyl orange, thymol blue, bromothymol blue, or neutral red, or m-cresol purple.

In certain example embodiments, the signal reagent is capable of producing a signal detectable by the naked eye, an optical sensor, a camera, a smartphone, a tablet, an electronic portable device, or any combination thereof.

In certain example embodiments, the one or more primer sets configured for LAMP amplification is/are specific for one or more the shrimp or fish pathogens selected from: White Spot Syndrome Virus (WSSV), Enterocytozoon hepatopenaei (EHP), Necrotising Hepatopancreatitis/Hepatobacter penaei, NHP-B), Infectious Hypodermal and Hematopoietic Necrosis Virus (IHHNV), Infectious Myonecrosis Virus (IMNV), Vibrio spp.-causing Acute hepatopancreatic necrosis disease (AHPND)/EMS), Yellow Head Virus (YHV), Taura Syndrome Virus (TSV), Vibrio parahaemolyticus, Tilapia Lake virus (TiLV), or any combination thereof.

In certain example embodiments, one or more primers of the one or more primer sets is selected from one or more of those set forth or otherwise identified in any one or more of Tables 1 (SEQ ID NOs: 9-14), 3 (SEQ ID NOS: 17-24), 4 (SEQ ID NOS: 25-32), 5 (SEQ ID NOS: 33-40), 6 (SEQ ID NOS: 41-48), 7 (SEQ ID NOS: 49-56), 8 (SEQ ID NOS: 57-64), 9 (SEQ ID NOS: 65-72), 10 (SEQ ID NOS: 73-80), 11A (SEQ ID NOS: 81-88), 11B (SEQ ID NOS: 125-126, 129-134), 12 (SEQ ID NOS: 89-96), 13 (SEQ ID NOS: 135-136, 139-144), 14 (SEQ ID NOS: 146-147, 150-155), 15 (SEQ ID NOS: 157-158, 161-166), 16 (SEQ ID NOS: 168-169, 172-177), and/or 17 (SEQ ID NOS: 179-180, 183-188) and/or FIGS. 8A(SEQ ID NOS: 17-24)-8B(SEQ ID NO: 97), 9A(SEQ ID NOS: 65-72)-9B(SEQ ID NO: 98):, 10A(SEQ ID NOS: 33-40)-10B(SEQ ID NO: 99), 11A(SEQ ID NOS: 49-56)-11B(SEQ ID NO: 100), 12A(SEQ ID NOS: 81-88, 101-102)-12B(SEQ ID NO: 103), 13A(SEQ ID NOS: 25-27, 29-32, 38)-13B(SEQ ID NO: 104), 14A(SEQ ID NOS: 89-96)-14B(SEQ ID NO: 105), and 15A(SEQ ID NOS: 109-116)-15B(SEQ ID NO: 106), 16A(SEQ ID NOS: 117-124)-16B(SEQ ID NO: 107), 17A(SEQ ID NOS: 73-80)-17B(SEQ ID NO: 196), 18A(SEQ ID NOS: 125-134)-18B(SEQ ID NO: 108), 19A(SEQ ID NOS: 135-144)-19B(SEQ ID NO: 145), 20A(SEQ ID NOS: 146-155)-20B(SEQ ID NO: 156), 21A(SEQ ID NOS: 157-166)-21B(SEQ ID NO: 167), 22A(22A SEQ ID NOS: 168-177)-22B(SEQ ID NO: 178), and/or 23A(SEQ ID NOS: 179-188)-23B(SEQ ID NO: 189).

In certain example embodiments, the one or more primer sets is/are configured to amplify a region of any 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more consecutive nucleotides in a polynucleotide that is 80, 81, 82, 83, 84, 85, 86, 87, 88, 89, 90, 91, 92, 93, 94, 95, 96, 97, 98, 99, to/or 100 percent identical to any one or more of SEQ ID NOs: 1-8, 97-108, 145, 156, 167, 178, 189-196 or any sequences complementary thereto.

Described in certain example embodiments herein are assays capable of detecting one or more shrimp pathogens comprising amplifying one or more shrimp or fish pathogen-specific target polynucleotides in a sample using loop-mediate isothermal amplification (LAMP), wherein amplification of the one or more shrimp or fish pathogen-specific target nucleotides generates a detectable signal; and detecting and/or measuring the detectable signal thereby determining the presence or absence of a shrimp or fish pathogen.

In certain example embodiments, the detectable signal is produced by a pH sensitive reagent that changes color from a first visible color to a second visible color in response to amplification of a target polynucleotide.

In certain example embodiments, the first visible color and the second visible color are different colors.

In certain example embodiments, the pH sensitive indicator changes color when the LAMP amplification moves from a neutral or basic pH to an acid pH as the reaction proceeds.

In certain example embodiments, the pH sensitive indicator is phenol red, cresol red, phenolphthalein, methyl orange, thymol blue, bromothymol blue, or neutral red, or m-cresol purple.

In certain example embodiments, the detectable signal is visible to the naked eye.

In certain example embodiments, detecting and/or measuring comprises imaging or otherwise analyzing the LAMP reaction with a smartphone, tablet, or other portable electronic device.

In certain example embodiments, LAMP is performed using one or more primer sets configured for LAMP amplification and specific for one or more the shrimp or fish pathogens selected from: White Spot Syndrome Virus (WSSV). Enterocytozoon hepatopenaei (EHP), Necrotising Hepatopancreatitis/Hepatobacter penaei (NHP-B), Infectious Hypodermal and Hematopoietic Necrosis Virus (IHHNV), Infectious Myonecrosis Virus (IMNV), Vibrio spp.-causing Acute hepatopancreatic necrosis disease (AHPND)/EMS) Yellow Head Virus (YHV), Taura Syndrome Virus (TSV), Vibrio parahaemolyticus, Tilapia Lake virus (TILV), or any combination thereof.

In certain example embodiments, one or more primers of the one or more primer sets is selected from one or more of those set forth or otherwise identified in any one or more of Tables 1 (SEQ ID NOs: 9-14), 3 (SEQ ID NOS: 17-24), 4 (SEQ ID NOS: 25-32), 5 (SEQ ID NOS: 33-40), 6 (SEQ ID NOS: 41-48), 7 (SEQ ID NOS: 49-56), 8 (SEQ ID NOS: 57-64), 9 (SEQ ID NOS: 65-72), 10 (SEQ ID NOS: 73-80), 11A (SEQ ID NOS: 81-88), 11B (SEQ ID NOS: 125-126, 129-134), 12 (SEQ ID NOS: 89-96), 13 (SEQ ID NOS: 135-136, 139-144), 14 (SEQ ID NOS: 146-147, 150-155), 15 (SEQ ID NOS: 157-158, 161-166), 16 (SEQ ID NOS: 168-169, 172-177), and/or 17 (SEQ ID NOS: 179-180, 183-188) and/or FIGS. 8A(SEQ ID NOS: 17-24)-8B(SEQ ID NO: 97), 9A(SEQ ID NOS: 65-72)-9B(SEQ ID NO: 98):, 10A(SEQ ID NOS: 33-40)-10B(SEQ ID NO: 99), 11A(SEQ ID NOS: 49-56)-11B(SEQ ID NO: 100), 12A(SEQ ID NOS: 81-88, 101-102)-12B(SEQ ID NO: 103), 13A(SEQ ID NOS: 25-27, 29-32, 38)-13B(SEQ ID NO: 104), 14A(SEQ ID NOS: 89-96)-14B(SEQ ID NO: 105), and 15A(SEQ ID NOS: 109-116)-15B(SEQ ID NO: 106), 16A(SEQ ID NOS: 117-124)-16B(SEQ ID NO: 107), 17A(SEQ ID NOS: 73-80)-17B(SEQ ID NO: 196), 18A(SEQ ID NOS: 125-134)-18B(SEQ ID NO: 108), 19A(SEQ ID NOS: 135-144)-19B(SEQ ID NO: 145), 20A(SEQ ID NOS: 146-155)-20B(SEQ ID NO: 156), 21A(SEQ ID NOS: 157-166)-21B(SEQ ID NO: 167), 22A(22A SEQ ID NOS: 168-177)-22B(SEQ ID NO: 178), and/or 23A(SEQ ID NOS: 179-188)-23B(SEQ ID NO: 189).

In certain example embodiments, the one or more primer sets is/are configured to amplify a region of any 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more consecutive nucleotides in a polynucleotide that is 80, 81, 82, 83, 84, 85, 86, 87, 88, 89, 90, 91, 92, 93, 94, 95, 96, 97, 98, 99, to/or 100 percent identical to any one or more of SEQ ID NOs: 1-8, 97-108, 145, 156, 167, 178, 189-196 or any sequences complementary thereto.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIGS. 1A-1B demonstrate specificity of WSSV LAMP in the detection of WSSV along with (FIG. 1A) other shrimp pathogens (FIG. 1B) shrimp related samples. P.v=DNA from Penaeus vannamei, P.m=DNA from Penaeus monodon. Red color indicates a negative reaction, yellow color (shown in greyscale) indicates a positive reaction

FIG. 2 demonstrates a serial dilution of VP28 plasmid by triplicate. Red color (shown in greyscale) indicates a negative reaction, yellow color (shown in greyscale) indicates a positive reaction.

FIG. 3 shows WSSV LAMP colorimetric results of 50 samples (1-50), ntc=non template control: +C=positive control. Red color indicates a negative reaction, yellow color indicates a positive reaction.

FIG. 4 shows a general workflow for a shrimp pathogen point of care assay described herein.

FIGS. 5A-5B show detection of (FIG. 5A) white spot syndrome virus (WSSV) and (FIG. 5B) infectious hypodermal and haematopoietic necrosis virus (IHHNV) and the clinical signs in shrimp of the respective diseases.

FIGS. 6A-6B show results from a LAMP isothermal method for shrimp bacterial pathogen detection. (FIG. 6A) Hepatobacter penaei/Necrotising hepatopancreatitis (NHP) detection targeting two different genes 16SrRNA & Flg E. (FIG. 6B) Vibrio parahaemolyticus causing-Acute hepatopancreatic necrosis disease (AHPND) detection targeting a toxin gene (Pir A). The clinical sign of shrimp affected by the corresponding diseases are shown alongside the pathogen detection panel by LAMP isothermal detection image.

FIG. 7A-7B show results from a LAMP isothermal method for detection of (FIG. 7A) a microsporidium Enterocytozoon hepatopenaei (EHP) (FIG. 7B): an internal control gene of shrimp.

FIGS. 8A-8B show primers and a map of the regions of WSSV VP28 gene amplified by LAMP amplification (FIG. 8A) Primers sequences: (FIG. 8B): Genomic region of WSSV VP28 gene amplified. Dark green indicates the forward primers. Light green indicates the backward primers.

FIGS. 9A-9B show primers and a map of the regions of IHHNV CP gene targeted for amplification by LAMP amplification (FIG. 9A) Primers sequences: (FIG. 9B): Genomic region of IHHNV gene amplified. Dark green indicates the forward primers. Light green indicates the backward primers.

FIGS. 10A-10B show primers and a map of the regions of NHP 16S rRNA gene used for LAMP amplification (FIG. 10A) Primers sequences: (FIG. 10B): Genomic region of NHP 16S rRNA gene amplified. Dark green indicates the forward primers. Light green indicates the backward primers.

FIGS. 11A-11B show primers and a map of the regions of NHP Flg E gene used for LAMP amplification (FIG. 11A) Primers sequences: (FIG. 11B): Genomic region of NHP FlgE gene amplified. Dark green indicates the forward primers. Light green indicates the backward primers.

FIGS. 12A-12B show primers and a map of the regions of AHPND Pir A region used for LAMP amplification (FIG. 12A) Primers sequences: (FIG. 12B): Genomic region of AHPND PirA gene amplified. Dark green indicates the forward primers. Light green indicates the backward primers.

FIGS. 13A-13B show primers and a map of the regions of EHP SSU region used for LAMP amplification (FIG. 13A) Primers sequences: (FIG. 13B): Genomic region of EHP SSU region amplified. Dark green indicates the forward primers. Light green indicates the backward primers.

FIGS. 14A-14B show primers and a map of the regions of shrimp 18srRNA. Primers and Map of the regions of shrimp penaeid 18srRNA internal control used for LAMP amplification (A) Primers sequences: (FIG. 14B): Genomic region of penaeid shrimp 18S rRNA gene amplified. Dark green indicates the forward primers. Light green indicates the backward primers.

FIGS. 15A-15B show a table of primers (FIG. 15A) and a map (FIG. 15B) of the genomic region of NHP 16S rRNA 2 gene amplified. Dark green indicates the forward primers. Light green indicates the backward primers.

FIGS. 16A-16B show a table of primers (FIG. 16A) and a map (FIG. 16B) of the region of an NHP gyrase B (GyrB) gene. FIG. 16B shows the genomic region of NHP Gyr B gene amplified during a LAMP assay. Dark green indicates the forward primers. Light green indicates the backward primers.

FIGS. 17A-17B show a table of primers (FIG. 17A) and a map (FIG. 17B) of the region of infectious myconecrosis virus (IMNV) capsid protein (CP) gene. FIG. 17B shows the genomic region of IMNV CP gene amplified during a LAMP assay. Dark green indicates the forward primers. Light green indicates the backward primers.

FIGS. 18A-18B show a table of primers (FIG. 18A) and a map (FIG. 18B) of the region of an AHPND pirB. FIG. 18B shows the genomic region of AHPND pirB gene amplified during a LAMP assay. Dark green indicates the forward primers. Light green indicates the backward primers.

FIGS. 19A-19B show a table of primers (FIG. 19A) and a map (FIG. 19B) of the region of an EHP Beta tubulin. FIG. 19B shows the genomic region of EHP Beta tubulin gene amplified during a LAMP assay. Dark green indicates the forward primers. Light green indicates the backward primers.

FIGS. 20A-20B show a table of primers (FIG. 20A) a map (FIG. 20B) of the region of an IMNV RdRp. FIG. 20B shows the genomic region of IMNV RNA-dependent-RNA polymerase (RdRp) gene amplified during a LAMP assay. Dark green indicates the forward primers. Light green indicates the backward primers.

FIGS. 21A-21B show a table of primers (FIG. 21A) and a map (FIG. 21B) of the region of a Vibrio parahaemolyticus ToxR. FIG. 21B shows the genomic region of Vibrio parahaemolyticus ToxR gene amplified during a LAMP assay. Dark green indicates the forward primers. Light green indicates the backward primers.

FIGS. 22A-22B show a table of primers (FIG. 22A) and a map (FIG. 22B) of the region of a Tilapia lake virus (TiLV) segment 3. FIG. 22B shows the genomic region of a Tilapia lake virus (TiLV) segment 3 gene amplified during a LAMP assay. Dark green indicates the forward primers. Light green indicates the backward primers.

FIGS. 23A-23B show a table of primers (FIG. 23A) and a map (FIG. 23B) of the region of a Tilapia lake virus (TiLV) segment 8. FIG. 23B shows the genomic region of a Tilapia lake virus (TiLV) segment 8 gene amplified during a LAMP assay. Dark green indicates the forward primers. Light green indicates the backward primers.

FIGS. 24A-24B show results demonstrating specificity (FIG. 24A) and sensitivity (FIG. 24B) in detecting AHPND targeting the pir B gene.

FIGS. 25A-25B show results demonstrating specificity (FIG. 25A) and sensitivity (FIG. 25B) in detecting EHP targeting the B-tubulin gene.

FIGS. 26A-26B show results demonstrating specificity (FIG. 26A) and sensitivity (FIG. 26B) in detecting NHP targeting the 16S rRNA2 gene.

FIGS. 27A-27B show results demonstrating specificity (FIG. 27A) and sensitivity (FIG. 27B) in detecting IMNV targeting the capsid protein (CP) gene.

FIGS. 28A-28B show results demonstrating specificity (FIG. 28A) and sensitivity (FIG. 28B) in detecting IHHNV targeting the capsid protein (CP) gene.

FIGS. 29A-29B show validation of AHPND detection targeting Pir B gene (FIG. 29A) and the sensitivity/specificity in detecting the AHPND Pir B gene (FIG. 29B).

FIGS. 30A-30B show validation of EHP detection targeting beta tubulin gene (FIG. 30A) and the sensitivity/specificity in detecting the beta tubulin gene (FIG. 30B).

FIGS. 31A-31B show validation of NHP detection targeting beta tubulin gene (FIG. 31A) and the sensitivity/specificity in detecting the 16S rRNA 2 gene (FIG. 31B).

FIGS. 32A-32B show validation of IMNV detection targeting beta tubulin gene (FIG. 32A) and the sensitivity/specificity in detecting the capsid protein (CP) gene (FIG. 32B).

FIGS. 33A-33C show validation of IHHNV detection targeting capsid protein (CP) gene in P. vannamei (FIG. 33A) and P. monodon and P. stylirostyris (FIG. 33B) and the sensitivity/specificity in detecting IHHNV CP gene (FIG. 33C).

FIGS. 34A-34B show validation of WSSV detection (FIG. 34A) and the sensitivity/specificity in detecting WSSV (FIG. 34B).

FIG. 35 show specificity in detecting Vibrio parahaemolyticus Tox R gene.

FIGS. 36A-36B show validation of WSSV detection (FIG. 36A) and the sensitivity/specificity in detecting WSSV (FIG. 36B) using a boiling method of DNA extraction.

FIGS. 37A-37B show validation of IMNV detection (FIG. 37A) and the sensitivity/specificity in detecting IMNV (FIG. 367) using a boiling method of DNA extraction.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis): Molecular Cloning: A Laboratory Manual, 4^{th} edition (2012) (Green and Sambrook): Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.): Benjamin Lewin, Genes IX, published by Jones and Bartlet, 2008 (ISBN 0763752223): Kendrew et al. (eds.), The Encyclopedia of Molecular Biology, published by Blackwell Science Ltd., 1994 (ISBN 0632021829): Robert A. Meyers (ed.), Molecular Biology and Biotechnology: a Comprehensive Desk Reference, published by VCH Publishers, Inc., 1995 (ISBN 9780471185710): Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

As used herein, “about,” “approximately.” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about.” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures. Biologic sample can also include biologic materials and even whole organisms that can be feed for shrimp, including, but not limited to krill, polycheates, blood worm, shrimp meal, Artemia, fishmeal, and fish oil. Such biological samples can be live or otherwise preserved such as be freezing.

The terms “subject,” “individual,” and “patient” are used interchangeably herein to refer to a vertebrate and non-vertebrate animals, including, but not limited to mammals and non-mammals. In some embodiments, the subject is a mammal. In some embodiments, the subject is a human, murine, simian, a farm animal (livestock, e.g., cows, pigs, chickens, sheep, goats, emu, bison, and the like), a sport animal (e.g., horses), a wild animal, or a pet animal (e.g., dog, cat, guinea pig, ferret, etc.). Non-mammal subjects include, but are not limited to, birds, fish, frogs, snakes, etc. In some embodiments, the subject is a non-human animal invertebrate including, but not limited to, a crustacean, e.g., a crab, lobster, or preferably a shrimp. Tissues, cells and their progeny of a biological entity obtained in vivo or cultured in vitro are also encompassed.

A “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

As used herein, “cDNA” refers to a DNA sequence that is complementary to a RNA transcript in a cell. It is a man-made molecule. Typically, cDNA is made in vitro by an enzyme called reverse-transcriptase using RNA transcripts as templates.

As used herein, “gene” refers to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA. For the purpose of this disclosure, genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. In some embodiments, a gene can be transcribed to yield non-coding RNA, such that the RNA has a functional role to play in the organism.

As used herein, “identity,” refers to a relationship between two or more nucleotide or polypeptide sequences, as determined by comparing the sequences. In the art, “identity.” can also refer to the degree of sequence relatedness between nucleotide or polypeptide sequences as determined by the match between strings of such sequences. “Identity.” can be readily calculated by known methods, including, but not limited to, those described in (Computational Molecular Biology, Lesk. A. M., Ed., Oxford University Press, New York, 1988; Biocomputing: Informatics and Genome Projects, Smith. D. W., Ed., Academic Press, New York, 1993; Computer Analysis of Sequence Data, Part I, Griffin. A. M., and Griffin. H. G., Eds., Humana Press, New Jersey, 1994; Sequence Analysis in Molecular Biology, von Heinje. G., Academic Press, 1987; and Sequence Analysis Primer, Gribskov. M. and Devereux. J., Eds., M Stockton Press, New York, 1991; and Carillo. H., and Lipman. D., SIAM J. Applied Math. 1988, 48:1073. Preferred methods to determine identity are designed to give the largest match between the sequences tested. Methods to determine identity are codified in publicly available computer programs. The percent identity between two sequences can be determined by using analysis software (e.g., Sequence Analysis Software Package of the Genetics Computer Group, Madison Wis.) that incorporates the Needelman and Wunsch, (J. Mol. Biol., 1970, 48:443-453) algorithm (e.g., NBLAST, and XBLAST). The default parameters are used to determine the identity for the polypeptides of the present disclosure, unless stated otherwise. Methods of determining identity can also be used to determine complementarity.

The term “molecular weight”, as used herein, generally refers to the mass or average mass of a material. If a polymer or oligomer, the molecular weight can refer to the relative average chain length or relative chain mass of the bulk polymer. In practice, the molecular weight of polymers and oligomers can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

As used herein, “negative control” can refer to a “control” that is designed to produce no effect or result, provided that all reagents are functioning properly and that the experiment is properly conducted. Other terms that are interchangeable with “negative control” include “sham,” “placebo,” and “mock.”

As used herein, “nucleic acid.” “nucleotide sequence,” and “polynucleotide” are used interchangeably herein and generally refer to a string of at least two base-sugar-phosphate combinations and refers to, among others, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotide as used herein can refer to triple-stranded regions comprising RNA or DNA or both RNA and DNA. The strands in such regions can be from the same molecule or from different molecules. The regions may include all of one or more of the molecules, but more typically involve only a region of some of the molecules. One of the molecules of a triple-helical region often is an oligonucleotide. “Polynucleotide” and “nucleic acids” also encompasses such chemically, enzymatically or metabolically modified forms of polynucleotides, as well as the chemical forms of DNA and RNA characteristic of viruses and cells, including simple and complex cells, inter alia. For instance, the term polynucleotide as used herein can include DNAs or RNAs as described herein that contain one or more modified bases. Thus, DNAs or RNAs including unusual bases, such as inosine, or modified bases, such as tritylated bases, to name just two examples, are polynucleotides as the term is used herein. “Polynucleotide”, “nucleotide sequences” and “nucleic acids” also includes PNAs (peptide nucleic acids), phosphonothioates, and other variants of the phosphate backbone of native nucleic acids. Natural nucleic acids have a phosphate backbone, artificial nucleic acids can contain other types of backbones, but contain the same bases. Thus, DNAs or RNAs with backbones modified for stability or for other reasons are “nucleic acids” or “polynucleotides” as that term is intended herein. As used herein, “nucleic acid sequence” and “oligonucleotide” also encompasses a nucleic acid and polynucleotide as defined elsewhere herein.

As used herein, “positive control” can refer to a “control” that is designed to produce the desired result, provided that all reagents are functioning properly and that the experiment is properly conducted.

As used herein, the term “specific binding” refers to covalent or non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸ M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “specifically detecting” refers to detecting a target polynucleotide predominantly while not substantially detecting non-target sequences at a level that detection (if any) of non-target sequences is undetectable or below the limit of detection or within background level of amplification for the particular method used.

As used herein, “specifically amplifying” refers to amplifying a target polynucleotide predominantly while not substantially amplifying to non-target sequences to a level that amplification (if any) of non-target sequences is undetectable or below the limit of detection or within background level of amplification for the particular method used.

As used herein, the term of art “primer” refers to a nucleic acid sequence that provides a starting point for DNA or RNA synthesis. They can be used in an in vitro reaction, such as in a DNA amplification method such as the polymerase chain reaction, a sequencing method, or in vitro transcription method. Primers are generally short (e.g. about 1-50 nucleotides) sequences, and are typically oligonucleotides. “Primer pair” refers to two primers that are designed to work together in a DNA synthesis and/or amplification method and can define the boundaries of a DNA (or RNA) being synthesized. Typically, there is a forward and reverse primer in a primer pair. Primers have complimentary sequences to a target polynucleotide. Generally, primers have a high degree of complementarity to a region in a target polynucleotide. This region is also referred to in the art as a “primer binding site.” The degree of complementarity between primer to a target polynucleotide can range from about 80-100%, such as 80 to 81, 82, 83, 84, 85, 86, 87, 88, 89, 90, 91, 92, 93, 94, 95, 96, 97, 98, 99, or 100%. The degree of complementarity between a primer to a target polynucleotide can be 81, 82, 83, 84, 85, 86, 87, 88, 89, 90, 91, 92, 93, 94, 95, 96, 97, 98, 99, or 100%.

As used herein, the term of art “amplification” refers to the production of additional polynucleotides via a method employing one or more primers and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. An exemplary amplification method is a PCR method.

As used herein, “tangible medium of expression” refers to a medium that is physically tangible or accessible and is not a mere abstract thought or an unrecorded spoken word. “Tangible medium of expression” includes, but is not limited to, words on a cellulosic or plastic material, or data stored in a suitable computer readable memory form. The data can be stored on a unit device, such as a flash memory or CD-ROM or on a server that can be accessed by a user via, e.g., a web interface.

As used herein, the terms “weight percent,” “wt %,” and “wt. %,” which can be used interchangeably, indicate the percent by weight of a given component based on the total weight of a composition of which it is a component, unless otherwise specified. That is, unless otherwise specified, all wt % values are based on the total weight of the composition. It should be understood that the sum of wt % values for all components in a disclosed composition or formulation are equal to 100. Alternatively, if the wt % value is based on the total weight of a subset of components in a composition, it should be understood that the sum of wt % values the specified components in the disclosed composition or formulation are equal to 100.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

Aquaculture farms, especially shrimp farms, is a rapidly growing market as they provide an abundant, cheap source of protein. The global shrimp market is currently valued at about $40 billion and is expected to increase to $68 billion by 2028. Of key concern to this market is aquaculture disease. Pathogens, such as bacterial, viral, and fungal pathogens, are a threat to the growth and expansion of the industry. An important aspect of pathogen control and treatment is testing and surveillance. As such there is urgent need for pathogen testing methods for aquaculture pathogens, particularly crustacean pathogens, and more particularly shrimp pathogens.

The current gold standard for detection of shrimp pathogens in the industry is conventional PCR-based detection of pathogen nucleic acids. However, the current techniques require expensive equipment that is not radially usable in the field, requires significant processing time, and highly skilled personnel to perform and interpret the tests. As such there is further need for improved testing methods for crustacean pathogens, particularly shrimp pathogens, that is rapid and is capable of field deployment.

With the limitations of current devices and techniques in mind, embodiments disclosed herein can provide assays and kits that can allow for, in some embodiments, rapid and easy to use and interpret testing for shrimp pathogens. The assays and kits herein can be scalable and used in the field without the need for special equipment or highly skilled labor. Described in some embodiments herein are assays and kits specific to shrimp pathogens that utilize loop-mediated isothermal amplification (LAMP), reverse transcription LAMP (RT-LAMP), and other LAMP-based amplification procedures (collectively referred to herein as “LAMP”, “LAMP amplification”, and the like). Loop-mediated isothermal amplification (LAMP) was developed for DNA detection by Notomi in 2000 (Notomi T, Okayama H, Masubuchi H, Yonekawa T, Watanabe K, Amino N, Hase T (2000) Loop-mediated isothermal amplification of DNA. Nucleic Acids Res.). LAMP is a simple, selective and efficient detection method that amplifies nucleic acids using DNA polymerase, being carried out in isothermal conditions with no complex lab equipment needed (Notomi et al. 2000; Parida M M, Santhosh S R, Dash P K, Tripathi N K, Lakshmi V, Mamidi N, Shrivastva A, Gupta N, Saxena P, Pradeep Babu J, Lakshmana Rao P V., Morita K (2007) Rapid and real-time detection of Chikungunya virus by reverse transcription loop-mediated isothermal amplification assay. J Clin Microbiol.; Das A, Babiuk S, McIntosh MT (2012) Development of a loop-mediated isothermal amplification assay for rapid detection of capripoxviruses. J Clin Microbiol.). The LAMP results are highly reliable and selective as the target DNA can be recognized by six distinct sequences (Notomi et al. 2000). Its high efficiency is due to the use of a single-step test tube at around 60-65° C. for approximately thirty minutes (Parida et al. 2007, Lu R, Wu X, Wan Z, Li Y, Jin X, Zhang C (2020) A novel reverse transcription loop-mediated isothermal amplification method for rapid detection of sars-cov-2. Int J Mol Sci.).

In some embodiments, the kits and assays are configured to provide diagnostic tests that can be completed in the field in a reasonably short time period (a couple of hours or less) and do not require delivery to a central processing facility for analysis. In some embodiments, detection of a positive or negative test result can be interpreted visually by the naked eye and/or readily available detection device, such as a smart phone or other personal portable smart device (e.g., smart watch, tablet, and the like). Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Kits

Described herein are kits composed of one or more reagents configured to detecting the presence of a target nucleic acid in a sample, where the target nucleic acid is a shrimp pathogen nucleic acid. In some embodiments, the kits are composed of one or more reagents configured for amplification, such as specific amplification, of one or more target nucleic acids in a sample, where the one or more target nucleic acids are for a shrimp pathogen and/or one or more suitable controls. In some embodiments, the kits are composed of one or more reagents configured for LAMP amplification, such as specific LAMP amplification, of one or more target nucleic acids in a sample, where the one or more target nucleic acids are for a shrimp pathogen and/or one or more suitable controls. In some embodiments, the kit includes, primers, reaction reagents and/or additives, buffers, signal reagents (i.e., reagent capable of reacting to produce a detectable signal indicative of a reaction condition, state, completion, and/or a result (e.g., a positive result, negative result, or inconclusive result), including, but not limited to, colormetric indicators, pH indicators, turbidity indicators, and the like), and the like to be used with an isothermal amplification technique, preferably a LAMP amplification. The one or more reagents can include reagents suitable for isothermal amplification, such as loop-mediated isotherm amplification (LAMP) or reverse transcription LAMP (RT-LAMP), and/or other LAMP-based amplification procedures (collectively referred to herein as “LAMP”, “LAMP amplification”, and the like).

In some exemplary embodiments herein, the kits include amplification reagents and a set of primers. In certain example embodiments, a kit further includes a lysis reagent. In certain example embodiments, the kit includes a signal reagent. In certain example embodiments, the kit comprises a sample collection component. In certain example embodiments, the kit includes a pre-mixed combination of amplification and/or signal reagents, and one or more primers and/or primer sets. In certain example embodiments, the kit includes a reaction vessel comprising a pre-mixed combination of amplification reagent(s) and/or signal reagent(s), and one or more primers and/or primer sets. In certain example embodiments the reaction vessel is designed to receive a sample or the sample collection component and be sealed. In some embodiments, the amplification, particularly LAMP amplification and signal generation and detection of one or more individual samples can be done at the point-of-care. In other embodiments, or the reaction vessel containing the collected sample and optionally the sample collection component can be sent to a central processing facility for performing the LAMP amplification and analysis. In certain examples, embodiment the kit further comprises one or more control sequences that can be amplified by the at least one of the primer sects in the kit. The control may be included in the pre-mixed solution with the amplification reagent(s) and/or the primer set(s).

### Primers

In some embodiments, the kit includes one or more primer sets where each primer set includes two or more primers. In some embodiments, two primers of the two or more primers of a primer set forms a primer pair. A primer pair refers to primers that are each capable of hybridizing to different sequences of a nucleic acid and are designed and configured to together define the region of the nucleic acid (e.g., DNA) that is amplified during an amplification reaction, such as a PCR or PCR-based amplification reaction, preferably a LAMP amplification. Any given primer set can have one, two, or more primer pairs. For example, a primer set having 6 primers, can include 1, 2, 3, or more primer pairs. It will be appreciated that a first primer can form a primer pair with more than one second primers. For example, a forward primer can form a primer pair with multiple different reverse primers. The primer pairs in primer sets having multiple primer pairs can be configured to amplify different and/or overlapping nucleic acid regions.

In some embodiments, the kit includes one or more primer sets including two or more primers, where the one or more primer sets are configured to amplify one or more target sequences in the sample in at least one LAMP amplification step. In some embodiments, the primers present in the kit are included in the kit at equal amounts or concentrations.

In some embodiments, the kit includes at least one primer set that is configured for loop-mediated isothermal amplification (LAMP) or reverse transcription loop-mediated isothermal amplification (RT-LAMP) and comprises at least one forward inner primer (FIP), backward inner primer (BIP), or both. In some embodiments, one, two, or more primers in a primer set includes one or more barcodes. In some embodiments, the barcode(s) is/are inserted between the two target-specific sequences of either the FIP, the BIP, or both.

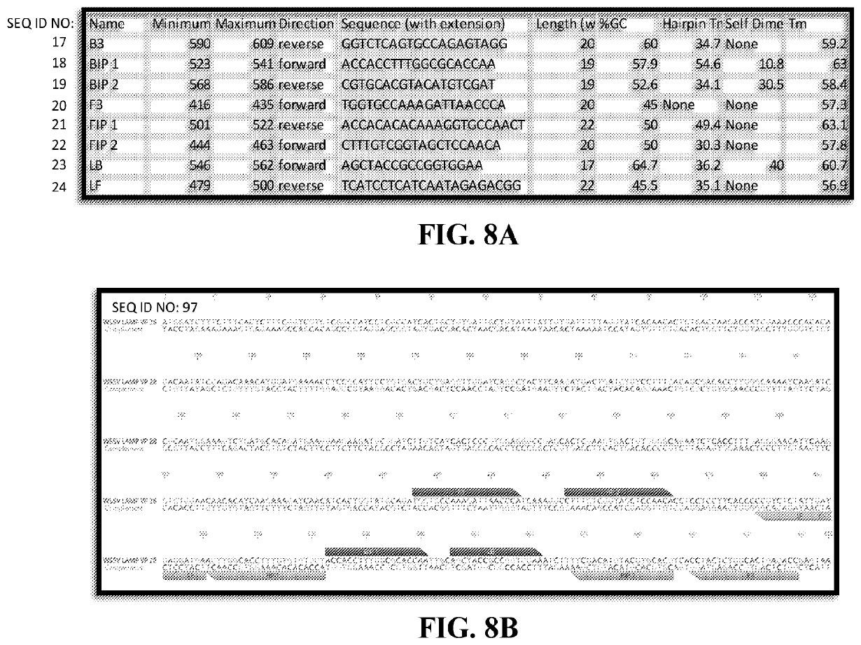

Primers that can be included in the kit specific to detect a shrimp pathogen via a LAMP amplification can be 90, 91, 92, 93, 94, 95, 96, 97, 98, 99, to/or 100 percent identical to any one or more of the primers shown in any one or more of Tables 1 (SEQ ID NOs: 9-14), 3 (SEQ ID NOS: 17-24), 4 (SEQ ID NOS: 25-32), 5 (SEQ ID NOS: 33-40), 6 (SEQ ID NOS: 41-48), 7 (SEQ ID NOS: 49-56), 8 (SEQ ID NOS: 57-64), 9 (SEQ ID NOS: 65-72), 10 (SEQ ID NOS: 73-80), 11A (SEQ ID NOS: 81-88), 11B (SEQ ID NOS: 125-126, 129-134), 12 (SEQ ID NOS: 89-96), 13 (SEQ ID NOS: 135-136, 139-144), 14 (SEQ ID NOS: 146-147, 150-155), 15 (SEQ ID NOS: 157-158, 161-166), 16 (SEQ ID NOS: 168-169, 172-177), and/or 17 (SEQ ID NOS: 179-180, 183-188) and/or FIGS. 8A(SEQ ID NOS: 17-24)-8B(SEQ ID NO: 97), 9A(SEQ ID NOS: 65-72)-9B(SEQ ID NO: 98):, 10A(SEQ ID NOS: 33-40)-10B(SEQ ID NO: 99), 11A(SEQ ID NOS: 49-56)-11B(SEQ ID NO: 100), 12A(SEQ ID NOS: 81-88, 101-102)-12B(SEQ ID NO: 103), 13A(SEQ ID NOS: 25-27, 29-32, 38)-13B(SEQ ID NO: 104), 14A(SEQ ID NOS: 89-96)-14B(SEQ ID NO: 105), and 15A(SEQ ID NOS: 109-116)-15B (SEQ ID NO: 106), 16A(SEQ ID NOS: 117-124)-16B(SEQ ID NO: 107), 17A(SEQ ID NOS: 73-80)-17B(SEQ ID NO: 196), 18A(SEQ ID NOS: 125-134)-18B(SEQ ID NO: 108), 19A(SEQ ID NOS: 135-144)-19B(SEQ ID NO: 145), 20A(SEQ ID NOS: 146-155)-20B(SEQ ID NO: 156), 21A(SEQ ID NOS: 157-166)-21B(SEQ ID NO: 167), 22A(22A SEQ ID NOS: 168-177)-22B(SEQ ID NO: 178), and/or 23A(SEQ ID NOS: 179-188)-23B(SEQ ID NO: 189. In some embodiments, one or more of the primer sets included in the kit can amplify one or more regions of a target nucleic acid. In some embodiments, the target nucleic acid has a sequence 80, 81, 82, 83, 84, 85, 86, 87, 88, 89, 90, 91, 92, 93, 94, 95, 96, 97, 98, 99, to/or 100 percent identical to any one or more of SEQ ID NOs: 1-8, 97-108, 145, 156, 167, 178, 189-196 or a sequence complementary thereto.

Each primer pair can amplify different or overlapping target sequences of a target polynucleotide (or nucleic acid). Example target polynucleotides are discussed in further detail elsewhere herein. Kits can be configured to detect a single target polynucleotide or more than one target polynucleotide.

Primers can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, to/or 40 or more nucleotides in length. Primers included in the kit and utilizes in the assay described herein can specifically bind to a polynucleotide, such a polynucleotide that is 80, 81, 82, 83, 84, 85, 86, 87, 88, 89, 90, 91, 92, 93, 94, 95, 96, 97, 98, 99, to/or 100 percent identical to any one or more of SEQ ID NOs: 1-8, 97-108, 145, 156, 167, 178, 189-196 or any sequences complementary thereto.

In some embodiments, the kit includes one or more primer sets configured to specifically amplify one or more target polynucleotides present in and/or unique to one or more shrimp or fish pathogens. In some embodiments, the kit includes one or more primer sets configured to specifically amplify one or more target polynucleotides present in and/or unique to White Spot Syndrome Virus (WSSV), Enterocytozoon hepatopenaei (EHP), Necrotising Hepatopancreatitis/Hepatobacter penaei (NHP-B), Infectious Hypodermal and Hematopoietic Necrosis Virus (IHHNV), Infectious Myonecrosis Virus (IMNV), Vibrio spp.-causing Acute hepatopancreatic necrosis disease (AHPND)/EMS), Yellow Head Virus (YHV), Taura Syndrome Virus (TSV), Vibrio parahaemolyticus, Tilapia Lake virus (TiLV), or any combination thereof. In some embodiments, the LAMP amplification reagents includes primers configured for LAMP and to specifically amplify one or more regions of one or more genes specific for one or more shrimp pathogens and/or for one or more suitable controls. In some embodiments, the LAMP amplification reagents includes primer to an internal control such as 18srRNA from the internal control Penaeus spp. In some embodiments, the LAMP amplification reagents includes one or more primer sets.

### Target Polynucleotides

In certain example embodiments, the target polynucleotides (or target nucleic acids) that can be detected by kits and assays described herein are present in and/or unique to one or more shrimp and/or fish pathogens, such as White Spot Syndrome Virus (WSSV), Enterocytozoon hepatopenaei (EHP), Necrotising Hepatopancreatitis/Hepatobacter penaei (NHP-B), Infectious Hypodermal and Hematopoietic Necrosis Virus (IHHNV), Infectious Myonecrosis Virus (IMNV), Vibrio spp.-causing Acute hepatopancreatic necrosis disease (AHPND)/EMS), Yellow Head Virus (YHV), Taura Syndrome Virus (TSV), Vibrio parahaemolyticus, Tilapia Lake virus (TiLV), or any combination thereof. In some embodiments, the target polynucleotides are one or more regions and/or are capable of amplifying a region (such as in an assay described herein) of a gene or noncoding region present in or unique to one or more shrimp and/or fish pathogens, such as White Spot Syndrome Virus (WSSV), Enterocytozoon hepatopenaei (EHP), Necrotising Hepatopancreatitis/Hepatobacter penaei (NHP-B), Infectious Hypodermal and Hematopoietic Necrosis Virus (IHHNV), Infectious Myonecrosis Virus (IMNV), Vibrio spp.-causing Acute hepatopancreatic necrosis disease (AHPND)/EMS), Yellow Head Virus (YHV), Taura Syndrome Virus (TSV), Vibrio parahaemolyticus, Tilapia Lake virus (TiLV), or any combination thereof. In some embodiments, the target polynucleotides are present in any one or more of SEQ ID NOs: 1-8, 97-108, 145, 156, 167, 178, 189-196. In some embodiments, the target polynucleotides are any 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more consecutive nucleotides in a polynucleotide that is 80, 81, 82, 83, 84, 85, 86, 87, 88, 89, 90, 91, 92, 93, 94, 95, 96, 97, 98, 99, to/or 100 percent identical to any one or more of SEQ ID NOs: 1-8, 97-108, 145, 156, 167, 178, 189-196 or any sequences complementary thereto.

The primer(s) can be included in the kit at any suitable concentration. In some embodiments, the concentration of each primer can be independently determined and can be the same or different as between two or more or all of the primers in the kit. The concentration of each primer can 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000, nM, μM, or mM, or more.

In some embodiments, the primers and/or primer sets are optimized so as to have a limit of detection (LOD) in an assay described herein of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, to/or 1000 target molecules or pathogens present per nL, μL, mL, ng, μg, mg, of total subject, starting sample, reaction, or processed sample.

### Amplification Reagents

In some embodiments, the kit includes one or more amplification reagents. Amplification reagents and systems known in the art can be designed and configured for use with the methods and systems detailed herein. In certain example embodiments, the RNA or DNA amplification is an isothermal amplification. In certain example embodiments, the isothermal amplification is LAMP amplification. The isothermal amplification kits and assays can be designed, via the selection of appropriate polymerases and buffers, to work over a wide range of temperatures. In some embodiments, the reagents are configured to work over or at a temperature of 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, to/or 75 degrees C.

In some embodiments, the amplification reagents can include one or more enzymes (e.g., polymerases), salts, or other buffers suitable for use and operation of a LAMP amplification. In some embodiments, the amplification reagent includes a salt, such as magnesium chloride (MgCl₂), potassium chloride (KCl), or sodium chloride (NaCl).

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. Polymerases that can be included in the kit that are suitable for a LAMP amplification or other isothermal amplification include, but are not limited to, a Bst polymerase I, Bst 2.0 (NE biolabs), WarmStart™ Bst 2.0 (NE Biolabs), Bst 3.0 (NEBiolabs), or full length Bst polymerase (NEbiolabs), a mesophilic polymerase (e.g., exo-variant of the E. coli DNA polymerase I Klenow fragment, the large fragment of Bsu DNA polymerase, and phi29 DNA polymerase. Others will be appreciated by one of ordinary skill in the art in view of this disclosure. One of skill in the art will be able to determine the optimum temperatures as appropriate for individual reagents.

### Signal Reagents

The kits can also include one or more reagents capable of developing a signal in response to a certain reaction condition or result. In some embodiments, the reaction indicator is a colorimetric or turbidimetric indicator. In some embodiments, the reaction indicator is a pH sensitive indicator. In some embodiments, the signal reagent(s) is/are configured to produce a detectable optical signal. The detectable optical signal can be detected by the naked eye and/or by a suitable optical sensing device. In some embodiments, the signal is such that it can be detected by a sensor/device such as that which can be included in a smart phone, tablet, or other portable device. In some embodiments, the signal can be analyzed and a result provided to a user through a computer program or application such as that which can be run on a smart phone, tablet, or other portable device. The detectable optical signal can be a wavelength of light or change in a wavelength of light (such as a reaction moves to completion) or a difference in wavelength of light (such as between a positive and negative result). In some embodiments, the wavelength(s) of light of the optical signal can be in the visual range and therefore can easily be detected visually by the naked eye. In some embodiments, the wavelengths of light of the optical signal can be in the infrared, Ultra Violet (UV) range. In some embodiments, the optical signal is a fluorescent signal.

In some embodiments, the optical signal is a change in turbidity (increasing or decreasing or no change in turbidity) of the sample.

Visual detection methods have been developed using different platforms including SYBR Green I (Nagamine et al. 2002: Dragan et al. 2012): hydroxynaphthol blue (HNB) (Goto et al. 2009) where propidium iodide turns pink in positive reactions (Hill et al. 2008) or orange in negative reactions (Subramaniam et al. 2014): change in turbidity (Das et al. 2012) and calcein (Tomita et al. 2008) among others. All of these can be adapted for use and/or inclusion with/in the present assays and kits. In the case of shrimp pathogens, several diagnostic methods have been developed to detect viral, bacterial and fungal diseases including histopathology (Lightner 1996), PCR, real-time PCR (OIE 2020). So far, isothermal methods for shrimp pathogens detection including RPA (Xia et al. 2014) and LAMP methods (Kono et al. 2004; Seetang-Nun et al. 2013: Jaroenram et al. 2009) have been developed. Some of these methods use lateral dipsticks (Jaroenram et al. 2009) which imply the use of an additional detection method. Two other visual detection method such as DNA-functionalized gold nanoparticles has been developed (Seetang-Nun et al. 2013) and LAMP with Mn-quenched calcein (Cao et al. 2020). Alternative detection methods for nucleic acid amplification use the color change of a metal-sensitive indicator, such as a shift from dark yellow to yellow (calcein) (Tomita et al. 2008), dark blue to blue (hydroxynaphthol blue) (Goto et al., 2009), or dark blue to light blue (malachite green). Some other methods require an additional step of adding ionic form of manganese (Jothikumar et al., 2014) which can increase the change of cross contamination. Other colorimetric methods such as Malachite green, features an additional color change in the negative reactions, with a change from dark blue to clear (Nzelu et al., 2014). These indicators require long incubation times (typically 60 min) and have been demonstrated with only moderate sensitivity (>100-1000 copies of target) (Animatsu et al., 2012). Intercalating nucleic acid dyes can be added to the reactions for real-time and visual detection, but clear visualization requires UV illumination (Goto et al., 2009). Although these methods and signal detection reagents can be adapted for use with the present kits and assays, it will be appreciated that these methods require an additional step of inclusion of the ionic form of manganese, an additional incubation period which increases the chance of cross contamination, and/or relies on a color change or shift that is difficult to discern by the naked eye or camera (e.g., a camera present on a smart phone, tablet, or other portable electronic device).

Thus, in some embodiments, the signal reagents produce a color change, which can be a clearly detectable color change (not just a change in color intensity or slight shift in color wavelength (e.g., green to green-blue), that optionally does not require the addition of additional reagents or require additional detection steps or methods. In some embodiments, the signal detection reagents that are pH sensitive and can change color based on pH change as the LAMP amplification proceeds. In some embodiments, the pH sensitive reagent that can change from one color to another (e.g., red to blue, yellow to red, etc.) that is easily discernable by the naked eye.

In some embodiments, as the LAMP reaction proceeds and the DNA polymerase incorporates a deoxynucleoside triphosphate into the nascent DNA, the released by-products include a pyrophosphate moiety and a hydrogen ion. The release of this proton has been the basis of this detection method in the presence of low concentrations of buffer and observed a significant change from an initial alkaline pH to a final acidic pH. See e.g., Tanner et al., 2015. BioTechniques. 58 (2): 59-68 doi 10.2144/000114253, which is incorporated by reference as if expressed in its entirety herein and can be adapted for use with and in view of the present disclosure.

In some embodiments, the concentration of the signal reagent included in the kit (or used in the methods/assays described in greater detail elsewhere herein) can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000, nM, μM, or mM.

Thus, in some embodiments, the kit includes one or more colorimetric, pH sensitive, or turbidity indicator such as any of those described herein and/or are otherwise generally known in the art. In some embodiments, the pH sensitive indicator is phenol red, cresol red, phenolphthalein, methyl orange, thymol blue, bromothymol blue, or neutral red, m-cresol purple.

In some embodiments, the signal, including optical signal, can be qualitative or is quantitative. For example, in some embodiments, the signal provides only a binary yes/no result (i.e., a target sequence is present or is not) but does not provide an indication of the amount or number of molecules of target present in the sample. In other embodiments, the signal can be quantitative and provide information to a user on the amount of target present in the sample, which can in some embodiments be indicative of pathogen load in the subject or environment from which the sample was obtained.

Lysis and/or DNA Extraction Reagents

Other components of a biological or chemical reaction may include a cell lysis component in order to break open or lyse a cell for analysis of the materials therein. A cell lysis component(s) may include, but are not limited to, a detergent, a salt as described above, such as NaCl, KCl, ammonium sulfate [(NH₄)₂SO₄], or others. Detergents that may be appropriate for the invention may include Triton X-100, sodium dodecyl sulfate (SDS), CHAPS (3-[(3-cholamidopropyl) dimethylammonio]-1-propanesulfonate), ethyl trimethyl ammonium bromide, nonyl phenoxypolyethoxylethanol (NP-40), Polysorbate 10, Polysorbate 20, Proteinase K, Saponin. Concentrations of detergents may depend on the particular application, and may be specific to the reaction in some cases.

### Additional Reagents

The kits can also include additional reagents, such as protein inhibitors, signal reagents capable of providing a detectable signal of reaction status and/or result, suitable control reagents, combinations thereof, and the like. In some embodiments, the control reagents are capable of amplifying a control target in a sample. Suitable control targets include housekeeping targets, or other targets that are expected to be expressed or are otherwise capable of being detected in all samples tested. In some embodiments, the suitable control targets are also typically expressed or otherwise capable of detection at about the same level or amount or at about the same relative level or relative amount (such as relative to sample weight or volume on a total, liquid, total protein, total nucleic acid, total DNA, total RNA, or dry matter basis).

### Sample Collection Component

The kit can include and/or the method can employ, in some embodiments, a sample collection component. The sample collection component may comprise one or more components to be used by a farmer or other worker. The sample collection may include a swab, punch biopsy, syringe, capillary, spoon, pick, spatula, straw, catheter, suction device, dropper, tissue, sample paper, tube scoop, tongs, scissors, scalpel and/or other precise or blunt dissection tool(s), for collection of sample from a subject, environment in which the subject lives or is otherwise cultivated, and/or a feed sample. The sample collection component can be sterilized and individually packaged within the kit. The sample collection component(s) can be configured for single use and can be disposable. The sample collection component(s) can be configured for re-use and/or re-sterilization. The sample collection component of the kit can include squirt bottles, bulbs, syringes or other means for rinsing of a sample, sampling area, cavity of a subject, etc. The bulb, bottle, or syringe may be provided pre-loaded with saline or other suitable liquid for the collection of a specimen. Jars, tubes, microtubes, and other sample containers can be included in the kit. The sample collection component can include further probes, swabs and tubes for further sample processing. In some embodiments, the sample collection component is configured for collection of a tissue sample from a shrimp, a feed sample, a water sample or other environmental sample (e.g., such as the environment that the shrimp lives or is cultivated in, or a combination thereof.

Sample collection components can include a vial, tube or other containment means for the collected sample. The sample collection component can include tubes, vials, containers or other receptacles for the collection of a sample wash. The containment means can include a lid configured to receive all or a portion of a swab, or can be configured such that the swab is provided as a portion of the lid to the containment means. The containment means can include one or more reagents, including amplification reagents, solvents, detergents and other solutions, and can be designed for use in further sample manipulations, shipping, and subsequent reactions. In some embodiments, one or more of the sample collection components is resistant to heat, and the collected sample can be further reacted and processed within the sample collection component, for example, for conducting the isothermal amplification reaction. Sample collection means can be further provided with ice packs and other shipping packaging.

In some embodiments, the sample collection component can be or include a sample dosing component. The sample dosing component can be configured to portion out or separate out a portion a collected sample to a desired amount (e.g., a dose appropriate for the downstream reaction(s)) and optionally facilitate placement of the portion in a reaction or other collection vessel. Non-limiting sample dosing components are spoons, spatulas, depression sticks, droppers, capillaries, syringes, and the like. In some embodiments, the sample dosing component can be part of or form the sample collection component. In some embodiments, the sample dosing component and the sample dosing component are separate components of the kit.

### Reaction Vessel

In some embodiments, the kit includes one or more reaction vessels. The reaction vessel can be one of the sample collection components described above, or may be a separate vessel suitable for the storing and shipping of a sample. The reaction vessel can include one or more pre-mixed ingredients, including amplification reagents, detergents, sterile solutions, and other reagents that may be utilized in further sample processing. In certain embodiments, the reaction vessel is configured to receive a sample collection component above, such as a swab. As an example, the reaction vessel can include a lid configured to receive all or a portion of a swab (or other sample collection component), or can be configured such that the swab is provided as a portion of the lid of the reaction vessel. The reaction vessel can be pre-loaded with sterile solution for the storing of the sample. More than one reaction vessel can be included if more than one target is to be detected. Each reaction vessel can include reagents specific for the detection of each separate target. Reagents (including primers) can be provided lyophilized, with reconstitution at the point of use, for example with a solution provided as a separate element of the kit. The reaction vessel can be designed to be heated and can be configured for use with particular heating elements and/or type of end-user.

The reaction vessel may be suitable for processing of individual samples or can include multi-well plates or tube strips capable of processing multiple samples simultaneously.

In some embodiments, the reaction vessel includes a pre-mixed combination of amplification reagents and primers and configured to be sealed after receiving the sample, sample collection component, sample dosing component, or a combination thereof.

In some embodiments, the reaction vessel is configured for use in an isothermal amplification reaction. In some embodiments, the reaction vessel is configured for use at a point of care. In some embodiments, the reaction vessel is configured for use in an isothermal amplification reaction conducted at a point of care.

### Methods

Also described herein are methods of shrimp pathogen detection using LAMP. In some embodiments, the methods allow for POC, rapid, and/or easy to visualize results so as to provide a practical diagnostic for shrimp pathogens that overcomes the limitations of current shrimp pathogen detection methods.

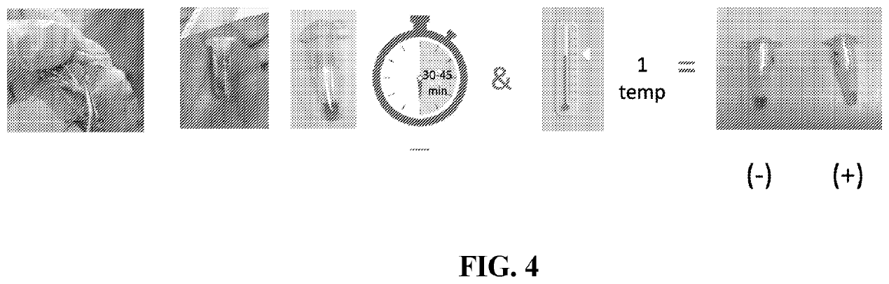

Generally, the method can include sample collection, sample processing (including lysis and other polynucleotide preparation, such as DNA extraction), LAMP amplification of target and/or control polynucleotides, and result analysis, such as by visualization or other suitable detection method. See e.g., FIG. 4. In some embodiments, DNA extraction is performed by digesting sample, such as cells or tissue samples, with a proteinase (e.g., Proteinase K), under suitable conditions, followed by a brief centrifugation (e.g., ˜6,000 RPM for about 5 seconds at about 25 degrees C.), followed by incubating the sample at about 65 degrees C. for about 15 minutes followed by incubating the sample at 95 degrees C. for about 15 minutes. This can be followed by centrifugation at about 10,500 rpm for about 1.5 minutes. See also Working Examples herein.

In certain example embodiments, a loop-mediated isothermal amplification (LAMP) reaction may be used to amplify target nucleic acids, which encompasses both LAMP and RT-LAMP reactions. LAMP can be performed with a four-primer system for isothermal nucleic acid amplification in conjunction with a polymerase. Notomi et al., Nucleic Acids Res. 2000, 28, 12, Nagamine et al., Molecular and Cellular Probes (2002) 16, 223-229, doi: 10.1006/mcpr.2002.0415. When performing LAMP with a 4-primer system, two loop-forming inner primers, denoted as FIP and BIP, are provided with two outer primers, F3 and B3. The inner primers each contain two distinct sequences, one for priming in the first stage of the amplification and the other sequence for self-priming in subsequent amplification states. The two outer primers initiate strand displacement of nucleic acid strands initiated from the FIP and BIP primers, thereby generating formation of loops and strand displacement nucleic acid synthesis utilizing the provided polymerase. LAMP can be conducted with two to six primers, ranging from only the two loop-forming primers, up to at least the addition of 2 additional primers, LF and LB along with the two outer primers and two inner primers. LAMP technologies advantageously have high specificity and can work at a variety of pH and temperature. In a preferred aspect, the LAMP is an isothermal reaction at between about 45° C. to 75° C., 55° C. to 70° C. or 60° C. to 65° C. Colorimetric LAMP (Y. Zhang et al., doi: 10.1101/2020.92.26.20028373), RT-LAMP (Lamb et al., doi: 10.1101/2020.02.19.20025155; and Yang et al., doi: 10.1101/2020.03.02.20030130) have been developed for detection of COVID-19, and are incorporated herein by reference in their entirety.

In certain embodiment, the LAMP reagents may include Bst 2.0+RTx or Bst 3.0 from New England Biolabs.

In an aspect, the primer sets for LAMP are designed to amplify one or more target sequences, generating amplicons that comprise the one or more target nucleic acids. In an aspect, a unique set of FIP (or BIP) primers are utilized in the LAMP reaction. The number of unique FIP (or BIP) primers in a set will vary based on assumptions for the assay to be conducted, for example, number of samples per run, targets amplified in any given reaction etc. In an aspect, the set of barcoded FIP (or BIP) primers in the kit include 1, 2, 3, 4, 5, 6, 7, 8, 9, 10, 11, 12, 13, 14, 15 or more FIP (or BIP) sequences. Use of the FIP (or BIP) primers can provide a resultant amplicon that preferably spans a fraction of the target nucleic acid sequence that is not covered by the amplification primers. In an aspect, the amplicon spans one or both junctions between a barcode sequence and the target nucleic acid sequence. In particular embodiments, the unique set of FIP (or BIP) primers can be utilized with the BIP (or FIP) primer, F3 primer, B3 primer, LF primer and LB primer. Upon heating of the sample to a temperature sufficient for LAMP amplification, e.g., 50° C.-72° C. (e.g., 50, 51, 52, 53, 54, 55, 56, 57, 58, 59, 60, 61, 62, 63, 64, 65, 66, 67, 68, 69, 70, 71, to/or 72° C.), using a polymerase and, optionally a reverse transcriptase (in the event RT-LAMP is utilized). Preferably the enzymes utilized in the LAMP reaction are heat-stabilized. One or more of the barcodes can be associated with the source of the sample, e.g., a patient identification, origin-specific barcode. Optionally, a control template is further provided with the sample, which may differ from the target sequence but share primer binding sites. In an aspect, the sample can continue to be heated subsequent to time sufficient to complete the LAMP reaction, to about 90° C. to about 100° C. (such as about 90, 91, 92, 93, 94, 95, 96, 97, 98, 99, to/or 100° C.), inactivating the enzymes and/or sterilizing the sample. The sample can be further processed through additional reactions, with or without a purification step. In some embodiments further processing can include signal development and/or detection.

Methods of amplifying, detection and/or quantifying using the systems disclosed herein can comprise incubating the sample or set of samples under conditions sufficient for an enzymatic reaction to occur. In certain example embodiments, the incubation time of the present invention may be shortened. One skilled in the art can perform biochemical reactions in 5 minutes (e.g., 5 minute ligation). Incubating may occur at one or more temperatures over time frames between about 10 minutes and 3 hours, preferably less than 200 minutes, 150 minutes, 100 minutes, 75 minutes, 60 minutes, 45 minutes, 30 minutes, or 20 minutes, depending on sample, reagents and components of the system. In some embodiments, the LAMP amplification occurs in a time of 2, 1.75, 1.5, 1.25, 1.0, 0.75, 0.5 hours or less.

In some embodiments, incubating, such as for LAMP, is performed at one or more temperatures between about 20° C. and 100° C., in certain embodiments or about 37° C., in some embodiments, between about 45° C. to 75° C., 55 to 70° C. or 60° C. to 65° C. In some embodiments, incubating is performed at one or more temperatures (such as in one or more incubation steps) at a temperature that is about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100° C. In some embodiments, incubation is performed in one or more steps where each step is performed at a temperature ranging from about 20, to about 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C. In some embodiments, incubation is performed in one or more steps where each step is performed at a temperature ranging from about 45, to 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75° C. In some embodiments, incubation is performed in one or more steps where each step is performed at a temperature ranging from about 55 to 56, 57, 58, 59, 60, 61, 62, 63, 64, 65, 66, 67, 68, 69, or 70° C. In some embodiments, incubation is performed in one or more steps where each step is performed at a temperature ranging from about 60 to 61, 62, 63, 64, or 65° C.

A salt, such as magnesium chloride (MgCl₂), potassium chloride (KCl), or sodium chloride (NaCl), may be included in an amplification reaction, such as PCR or LAMP, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Although the kits and methods described herein do not require the use of special equipment to be performed, this does not exclude one or more steps, in some embodiments, being performed on one or more pieces of equipment, such as a thermocycler. Thus, in some embodiments, LAMP amplification of nucleic acids may be performed using specific thermal cycle machinery or equipment, and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In some embodiments, the method includes reverse transcription of a target nucleic acid or a nucleic acid that includes a target nucleic acid.

Amplification reactions may include dNTPs and nucleic acid primers used at any concentration appropriate for the shrimp pathogen LAMP-based detection of the present disclosure, such as including, but not limited to, a concentration of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, to/or 1000, nM, μM, or mM or more. Likewise, a polymerase useful in accordance with the invention may be any specific or general polymerase known in the art and useful or the kits and assays, including a Bst polymerase or other polymerase capable of LAMP amplification and/or reverse transcription.

Exemplary methods are also demonstrated and described in the Working Examples below.

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1-Development of a Loop-Mediated Isothermal Amplification (LAMP) Assay for White Spot Syndrome Virus (WSSV) Detection in the Pacific White Shrimp (Penaeus Vannamei)

### 1. Introduction

The shrimp industry is one of the animal-producing sectors that has been continuously growing for the last 20 years from about 1 million metric tons (MMT) in 2000 up to about 5.5 MMT in 2017 (Anderson & Valderrama 2017, FAO 2020). The main limitation for this industry to grow is the presence of infectious diseases (Flegel et al. 2008: Lightner & Flegel 2012) and the lack of a rapid detection system that allow to detect a given pathogen at early stage. Among the shrimp diseases, viral and bacterial diseases are the one that cause more losses in the shrimp industry (Lightner 1996). White spot disease (WSD), caused by the white spot syndrome virus (WSSV) is the most important disease in the shrimp industry worldwide (Lightner 1996). This virus was first reported in China in 1992 and from there it spread to all of the Asian countries that cultured Penaeid shrimp (Takahashi et al. 1994: Chou et al. 1995). In 1995, WSSV was reported in the USA (Nunan et al. 1998: Durand et al. 2000) and during 1999-2000, White Spot Disease (WSD) had become established in most of the major shrimp farming countries in the Americas and SE Asia causing significant losses to the shrimp farming industry (Lightner 2011).

Loop-mediated isothermal amplification (LAMP) was developed for DNA detection by Notomi in 2000 (Notomi et al. 2000). LAMP is a simple, selective and efficient detection method that amplifies nucleic acids using DNA polymerase, being carried out in isothermal conditions with no complex lab equipment needed (Notomi et al. 2000, Parida et al. 2007, Das et al. 2012). The LAMP results are highly reliable and selective as the target DNA can be recognized by six distinct sequences (Notomi et al. 2000). Its high efficiency is due to the use of a single-step test tube at around 60-65° C. for approximately thirty minutes (Parida et al. 2007, Lu et al. 2020).

Visual detection methods have been widely developed using different platforms including SYBR Green I (Nagamine et al. 2002; (Dragan et al. 2012) hydroxynaphthol blue (HNB) (Goto et al. 2009) propidium iodide turns pink in positive reactions (Hill et al. 2008) or orange in negative reactions (Subramaniam et al. 2014), change in turbidity (Das et al. 2012) and calcein (Tomita et al. 2008) among others. In case of shrimp pathogens, several diagnostic methods have been developed to detect WSSV including histopathology (Lightner 1996)), PCR (Lo et al. 1996), real-time PCR (Durand & Lightner 2002). So far, isothermal methods for WSSV detection have been developed including RPA (Xia et al. 2014) and LAMP methods (Kono et al. 2004; Seetang-Nun et al. 2013; Jaroenram et al. 2009). Some of these methods use lateral dipsticks (Jaroenram et al. 2009) which imply the use of an additional detection method. Another visual detection method such as DNA-functionalized gold nanoparticles has been developed (Seetang-Nun et al. 2013) and LAMP with Mn-quenched calcein (Cao et al. 2020). However, these methods require an additional step of inclusion of the ionic form of manganese and an additional incubation period which increases the chance of cross contamination. In this Example, a pH change was employed to develop a novel colorimetric assay for WSSV detection using LAMP amplified product visualized by an naked eye is shown.

### 2. Materials and Methods

### 2.1 Shrimp, WSSV Inoculum and Challenge Test

The WSSV isolate used in this study was from a WSSV epizootic in China during 1993. This virus has been maintained by continuous transfer in SPF P. vannamei and stored at −70° C. at the APL-UA. The shrimp utilized in the study were obtained from a commercial shrimp facility in the United States. A total of 250 SPF P. vannamei, with an average weight of 10 g each, were used in the study.

A WSSV challenge test was conducted via oral route by feeding previously confirmed WSSV-infected minced shrimp tissue to SPF P. vannamei. This was done at 10% body weight of the SPF animals divided into two rations (AM and PM) in 1 day. At the end of the challenge shrimp were collected and frozen at −80° C. for further analysis.

### 2.2 DNA Extraction

In brief, DNA extraction was done on shrimp samples by pooling gills/pleopods using a Qiagen DNeasy Blood and Tissue Kit following the manufacturer's protocol. Following extraction, the DNA concentration was determined by a Nanodrop. Samples were aliquoted at 50 ng/ul and stored at −20° C. until further analysis.

### 2.3 LAMP

Loop-mediated isothermal amplification (LAMP) assay primers were designed from the WSSV vp28 gene using Primer Explorer V5 (Fujitso) (Table 1).

TABLE 1

| Pathogen | Primer | Position* | Sequence (5′ 3 ) |
|---|---|---|---|
| | F3 wssv | 249,285-249,305 | TGGTGCCAAAGATTAACCCA (SEQ ID NO: 9) |
| WSSV | B3 wssv | 249,478-249,459 | GGTCTCAGTGCCAGAGTAGG (SEQ ID NO: 10) |
| | FIP wswv | 249,391-249,370/ | ACCACACACAAAGGTGCCAACTCTTTGTC |
| | | 249,313-249,333 | GGTAGCTCCAACA (SEQ ID NO: 11) |
| | BIP wssv | 249,392-249,411/ | ACCACCTTTGGCGCACCAATCGTGCACGTA |
| | | 249,456-249,437 | CATGTCGA (SEQ ID NO: 12) |
| | LF wswv | 249,370-249,348 | TCATCCTCATCAATAGAGACGG (SEQ ID NO: 13) |
| | LB wssv | 249,415-249,432 | AGCTACCGCCGGTGGAA (SEQ ID NO: 14) |

*Based on the full-genome sequence AF332093

All LAMP reactions were performed following New England Biolab's recommended protocol using WarmStart Colorimetric LAMP 2X Master Mix (NEB, Massachusetts USA, M1800 L). Briefly, 20 μL reactions containing 10 μL LAMP master mix, 2 μL of 10X primer mix (2 μM F3 and B3, 16 μM Forward Inner Primer (FIP) and Backward Inner Primer (BIP), and 4 μM of Loop Forward (LF) and Loop Backward (LB) primers, 6 μL nuclease-free water, and 2 μL samples. LAMP reactions were incubated at 65° C. using BioRad DNA Engine thermocyclers for 30 minutes followed by a 2 min at 90° C. Photographs of samples laid on white sheets of paper were taken with cell phone cameras.

2.4 Quantitative Real-Time PCR (qPCR) Assay

Extracted DNA samples were amplified following OIE protocol for real-time PCR (Durand & Lightner, 2002). The real-time PCR amplifications were carried out with a StepOnePlus real-time PCR system (Life Technologies) using: 0.5 μL of each primer specific to WSSV, 0.1 μM of TaqMan probe, 1X TaqMan Fast virus 1-step Master Mix (Life Technologies), 20 ng of DNA and HPLC water in a reaction volume of 10 μL.

### 2.5 Generating a Standard Curve for Quantification of WSSVvp28

To generate a plasmid DNA standard curve for the WSSV vp28 DNA extracted from hemolymph of a WSSV-infected shrimp was used as a template to amplify the complete ORF of vp28 with specific primers: VP28 F (5′ GCG AAG CTT AAT GGA TCT TTC TTT C 3′ (SEQ ID NO: 15)) including a Hind III restriction site (underlined) and VP28R: (5′ GAC ACA TCT AGA TAC TCG GTC TCA G 3′ (SEQ ID NO: 16)) containing a Xba I restriction site (underlined). The insert was cloned in a pGEM-T vector (Promega) and then the VP28 DNA fragment was excised and cloned in pGFPuv vector (Clontech) by digestion with Hind III and Xba I. The resulting recombinant plasmid pGFPuv-VP28 and the control pGFPuv were transformed in E. coli JM 109 competent cells for propagation. The recombinant plasmid pWSSVvp28 was verified by DNA sequencing in both orientations with an automated Applied Biosystems 3730 DNA Analyzer. The concentration of pWSSVvp28 was determined by measuring the optical density (OD) at 260 nm in a Nanodrop™ spectrophotometer. The DNA copy number was obtained by using the formula: (Amount of DNA in ng) (Avogadro's number)/(650 Da) (length of template as bp) (Staroscik, 2004).

### 2.6 Specificity of WSSV LAMP Assays

DNA was extracted from SPF shrimp and shrimp infected with several shrimp diseases including white spot syndrome virus (WSSV), infectious hypodermal and haematopoietic necrosis virus (IHHNV), yellow head virus (YHV), infectious myonecrosis virus (IMNV), taura syndrome virus (TSV), Enterocytozoon hepatopenaei (EHP), Hepatobacter penaei (NHP) and V. parahaemolyticus causing-Acute hepatopancreatic necrosis disease (VPAHPND). A total of 50 ng of extracted DNA was subjected to LAMP reaction for evaluation of the specificity of WSSV LAMP assay.

### 2.7 Sensitivity of WSSV LAMP

Plasmid DNA containing the whole vp28 gene was serially diluted from 106 to 101 copies per triplicate to determine the sensitivity of the LAMP assay.

### 2.8 Validation in Clinical Samples

Total genomic DNA was isolated from a total of fifty shrimp Penaeus vannamei, were run by two methods real-time PCR, and LAMP. Fifty (50) ng of genomic DNA subjected to LAMP assay for WSSV detection. The DNA extracted from SPF shrimp was served as negative control. The detection of the WSSV by real-time PCR was performed following OIE method (OIE 2020).

### 2.9 Statistical Analysis

The calculation of the diagnostic sensitivity (DSe) and diagnostic specificity (DSp) (OIE, 2017) between the two methods was based on the following formula. DSe=TP/(TP+FN) and DSp=TN/(TN+FP) where TP means true-positive cases, FN means false-negative cases, TN means true-negative cases, and FP means false-positive cases.

### Results

### 3.1 Specificity of LAMP Assays

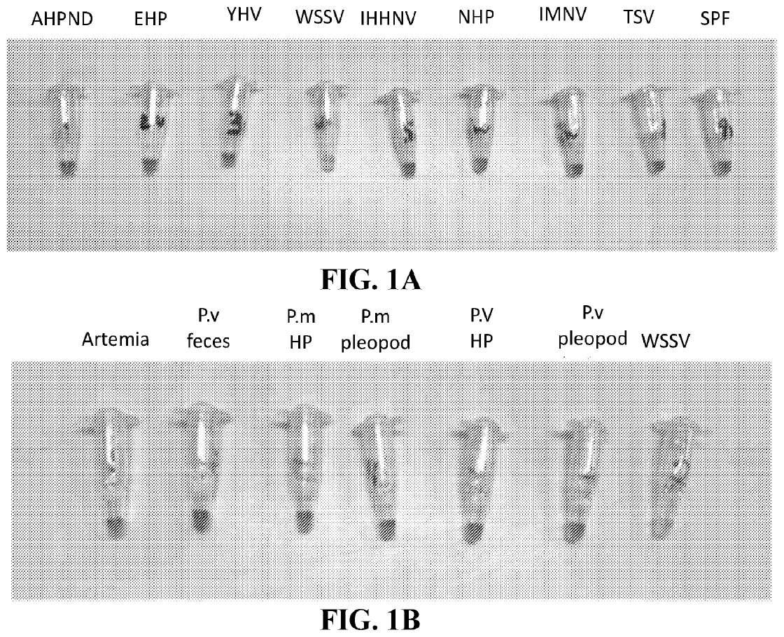

The specificity of WSSV-LAMP was determined through evaluation of cross-reactivity of DNA from SPF shrimp and shrimp infected with other pathogens. While a positive reaction was observed when DNA from WSSV-infected shrimp was used in the LAMP assay, no amplification was observed in DNA from SPF shrimp and shrimp infected with AHPND, EHP, YHV, IHHNV, NHP, IMNV, TSV (FIGS. 1A-1B).

### 3.2 Sensitivity of LAMP Assays

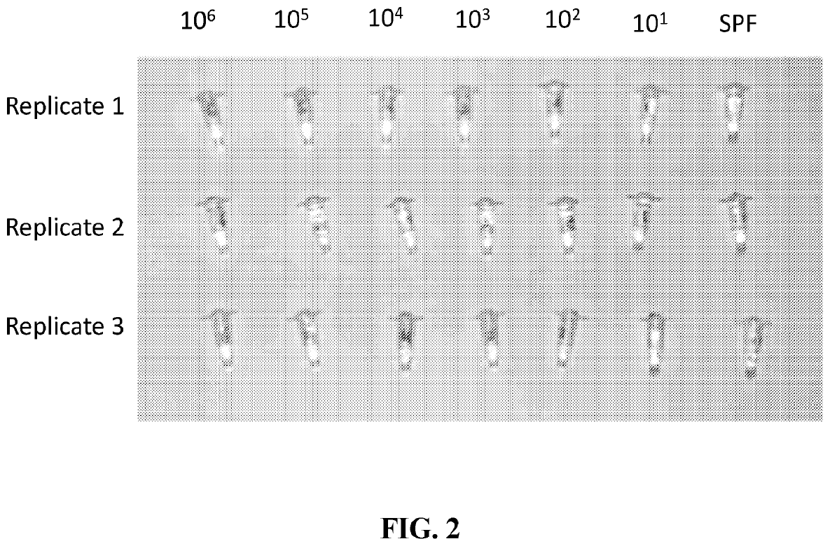

A serial dilution of plasmid from 106 to 100 copies were employed to determine the sensitivity of WSSV LAMP assay. The results showed that the detection limit of WSSV using the LAMP assay is 100 copies (FIG. 2).

### 3.3 Detection of Clinical Samples Using WSSV LAMP Assay

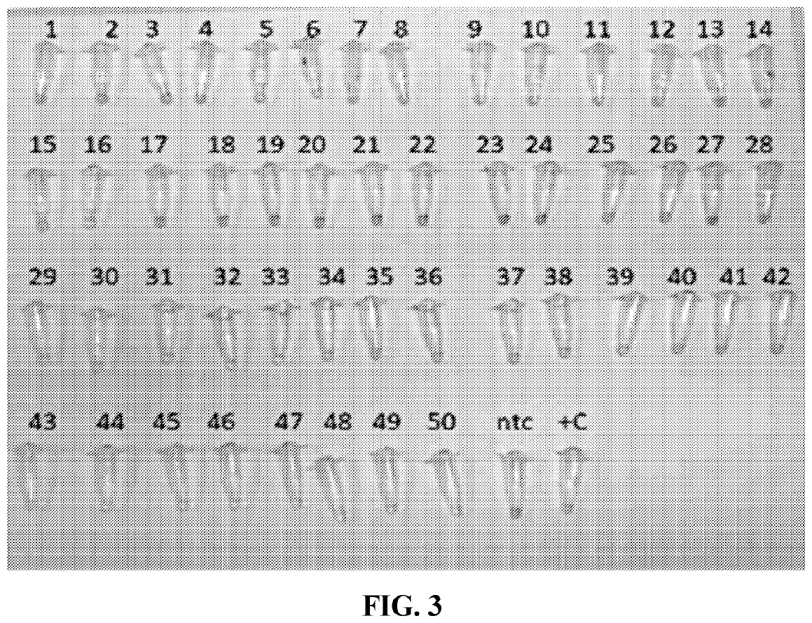

Shrimp samples from a WSSV challenge test were analyzed by both real-time PCR and LAMP for WSSV detection. FIG. 3 shows the colorimetric results of 50 samples (1-50), ntc-non template control: +C=positive control.

To determine the reliability of the LAMP assay in detecting WSSV in shrimp, DNA extracted from 50 samples including healthy animals were analyzed by real-time PCR and LAMP independently. The results showed that out of 38 WSSV-infected samples that tested positive for WSSV by real-time, 36 tested positive by LAMP. Twelve (12) negative samples tested not detected by both methods, real-time PCR and LAMP (Table 2). Table 2 shows a comparison of the tests for clinical samples by the LAMP and OIE method TaqMan-based qPCR show high similarity.

TABLE 2

| | | | | | Comparison of WSSV LAMP | |
|---|---|---|---|---|---|---|
| | | Real-time PCR | | | with real-time PCR | |
| | | Positive | Negative | Total | DSe* | DSp* |
| LAMP | Positive | 36 | 0 | 36 | 95% | 100% |
| | | | | | (83.08-99.39) | (73.54-100.00) |
| | Negative | 2 | 12 | 14 | | |
| | Total | 38 | 12 | 50 | | |

*DSe = Diagnostic sensitivity; DSp = Diagnostic specificity

The analytical sensitivity was compared with these two methods WSSV LAMP and the OIE real-time method. The analytical sensitivity was 95% of Dse for WSSV based LAMP platform.

### 4. Discussion

This Example demonstrates a method for WSSV detection through nucleic acid amplification by LAMP method using a colorimetric sensitive indicator based on pH. This assay allow to have a reliable result in 30 min with high specificity and high sensitivity. Unlike other LAMP-based methods, this method allow to have reliable results without the need of additional steps after the incubation period with a clear change of color from red to yellow. Alternative detection methods for nucleic acid amplification use the color change of a metal-sensitive indicator, such as a shift from dark yellow to yellow (calcein) (Tomita et al. 2008), dark blue to blue (hydroxynaphthol blue) (Goto et al., 2009), or dark blue to light blue (malachite green). These color shifts are difficult to discern by eye. Some other methods require an additional step of adding ionic form of manganese (Jothikumar et al., 2014) which can increase the change of cross contamination. Other colorimetric methods such as Malachite green, features an additional color change in the negative reactions, with a change from dark blue to clear (Nzelu et al., 2014). These indicators require long incubation times (typically 60 min) and have been demonstrated with only moderate sensitivity (>100-1000 copies of target) (Animatsu et al., 2012). Intercalating nucleic acid dyes can be added to the reactions for real-time and visual detection, but clear visualization requires UV illumination (Goto et al., 2009). In our assay, when a DNA polymerase incorporates a deoxynucleoside triphosphate into the nascent DNA, the released by-products include a pyrophosphate moiety and a hydrogen ion. The release of this proton has been the basis of this detection method in the presence of low concentrations of buffer and observed a significant change from an initial alkaline pH to a final acidic pH. Such a significant change in pH presented the possibility of detecting DNA amplification through the use of pH-sensitive indicator dyes, which we present here as a rapid, robust method for detecting LAMP amplification.

### REFERENCES FOR EXAMPLE 1

Anderson J L, Valderrama D (2017) Shrimp Production Review. Aquaculture Alliance
Arunrut N, Kampeera J, Sirithammajak S, Sanguanrut P, Proespraiwong P, Suebsing R (2016) Sensitive Visual Detection of AHPND Bacteria Using Loop-Mediated Isothermal Amplification Combined with DNA-Functionalized Gold Nanoparticles as Probes. PLOS One 11:151769.
Bell T A, Lightner D V (1988) A Handbook of Normal Penaeid Shrimp Histology, First Edit. World Aquaculture Society, Baton Rouge, Louisiana.
Cao Z, Dong X, Zou P Z, Wan X Y, Li C, Huang J (2020) Development of a cost-efficient micro-detection slide system for the detection of multiple shrimp pathogens. Anal Biochem.
Cruz-Flores R, Mai H N, Dhar A K (2019) Multiplex SYBR Green and duplex TaqMan real-time PCR assays for the detection of Photorhabdus Insect-Related (Pir) toxin genes pirA and pirB. Mol Cell Probes 43:20-28.
Das A, Babiuk S, McIntosh M T (2012) Development of a loop-mediated isothermal amplification assay for rapid detection of capripoxviruses. J Clin Microbiol.
Dragan A I, Pavlovic R, McGivney J B, Casas-Finet J R, Bishop E S, Strouse R J, Schenerman M A, Geddes C D (2012) SYBR Green I: Fluorescence properties and interaction with DNA. J Fluoresc.
Durand S V, Lightner D V (2002) Quantitative real time PCR for the measurement of white spot syndrome virus in shrimp. J Fish Dis 25:381-389.
FAO (2020) The state of world fisheries and Aquaculture. Food and Agriculture Organization of the United Nations, ROME-Italia.
Flegel T W, Lightner D V, Lo C F, Owens L (2008) Shrimp Disease Control: Past, Present and Future. In: Diseases in Asian Aquaculture VI. Bondad-Reantaso M G, Mohan C V, Crumlish M, Subasinghe (eds) Fish Health Section, Asian Fisheries Society, Manila, Philippines, p 355-378
Goto M, Honda E, Ogura A, Nomoto A, Hanaki K I (2009) Colorimetric detection of loop-mediated isothermal amplification reaction by using hydroxy naphthol blue. Biotechniques.
Hill J, Beriwal S, Chandra I, Paul V K, Kapil A, Singh T, Wadowsky R M, Singh V, Goyal A, Jahnukainen T, Johnson J R, Tarr P I, Vats A (2008) Loop-mediated isothermal amplification assay for rapid detection of common strains of Escherichia coli. J Clin Microbiol.
Jaroenram W, Kiatpathomchai W, Flegel T W (2009) Rapid and sensitive detection of white spot syndrome virus by loop-mediated isothermal amplification combined with a lateral flow dipstick. Mol Cell Probes.
Kanrar S, Dhar A K (2018) Complete genome sequence of a deletion mutant of Vibrio parahaemolyticus from Pacific white shrimp (Penaeus vannamei). Genome Announc 6.
Kongrueng J, Tansila N, Mitraparp-Arthorn P, Nishibuchi M, Vora G J, Vuddhakul V (2015) LAMP assay to detect Vibrio parahaemolyticus causing acute hepatopancreatic necrosis disease in shrimp. Aquac Int 23:1179-1188.
Kono T, Savan R, Sakai M, Itami T (2004) Detection of white spot syndrome virus in shrimp by loop-mediated isothermal amplification. J Virol Methods.
Kumar R, Ng T H, Wang H-C (2020) Acute hepatopancreatic necrosis disease in penaeid shrimp. Rev Aquac.
Lee C Te, Chen I T, Yang Y T, Ko T P, Huang Y T, Huang J Y, Huang M F, Lin S J, Chen C Y, Lin S S, Lightner D V., Wang H C, Wang A H J, Wang H C, Hor L I, Lo C F (2015) The opportunistic marine pathogen Vibrio parahaemolyticus becomes virulent by acquiring a plasmid that expresses a deadly toxin. Proc Natl Acad Sci USA 112: 10798-10803.
Lightner D, Flegel T (2012) Diseases of Crustaceans-Acute hepatopancreatic necrosis syndrome (AHPNS) (disease card). asia pacific emergency regional consultation on early mortality syndrome (EMS)/Acute hepatopancreatic necrosis syndrome (AHPNS).
Lightner DD V. (1996) A handbook of shrimp pathology and diagnostic procedures for diseases of cultured penaeid shrimp. Lousiana, Baton Rouge, Louisiana, USA.
Lightner DV (2012) Biology and Pathology of Early Mortality Syndrome of Shrimp. In: Global Outlook for Aquaculture Leadership. Bangkok, Thailand, p 40
Liu L, Jiang L, Yu Y, Xia X, Pan Y, Yan S, Wang Y (2017) Rapid diagnosis of Vibrio owensii responsible for shrimp acute hepatopancreatic necrosis disease with isothermal recombinase polymerase amplification assay. Mol Cell Probes 33:4-7.
Lo C F, Leu J H, Ho C H, Chen C H, Peng S E, Chen Y T, Chou C M, Yeh P Y, Huang C J, Chou H Y, Wang C H, Kou G H (1996) Detection of baculovirus associated with white spot syndrome (WSBV) in penaeid shrimps using polymerase chain reaction. Dis Aquat Organ.
Lu R, Wu X, Wan Z, Li Y, Jin X, Zhang C (2020) A novel reverse transcription loop-mediated isothermal amplification method for rapid detection of sars-cov-2. Int J Mol Sci.
Nagamine K, Hase T, Notomi T (2002) Accelerated reaction by loop-mediated isothermal amplification using loop primers. Mol Cell Probes.
Notomi T, Okayama H, Masubuchi H, Yonekawa T, Watanabe K, Amino N, Hase T (2000) Loop-mediated isothermal amplification of DNA. Nucleic Acids Res.
Parida M M, Santhosh S R, Dash P K, Tripathi N K, Lakshmi V, Mamidi N, Shrivastva A, Gupta N, Saxena P, Pradeep Babu J, Lakshmana Rao P V., Morita K (2007) Rapid and real-time detection of Chikungunya virus by reverse transcription loop-mediated isothermal amplification assay. J Clin Microbiol.
Seetang-Nun Y, Jaroenram W, Sriurairatana S, Suebsing R, Kiatpathomchai W (2013) Visual detection of white spot syndrome virus using DNA-functionalized gold nanoparticles as probes combined with loop-mediated isothermal amplification. Mol Cell Probes.
Subramaniam K, Shariff M, Omar A R, Hair-Bejo M, Ong B L (2014) Use of acridine orange to visually improve the loop-mediated isothermal amplification for detection of infectious spleen and kidney necrosis virus. Fish Pathol.
Tang K, Navarro S, Pantoja C, Aranguren F, Lightner D (2012) New genotypes of white spot syndrome virus (WSSV) and Taura syndrome virus (TSV) from the Kingdom of Saudi Arabia. Dis Aquat Organ 99:179-185.
Tomita N, Mori Y, Kanda H, Notomi T (2008) Loop-mediated isothermal amplification (LAMP) of gene sequences and simple visual detection of products. Nat Protoc.
Xia X, Yu Y, Weidmann M, Pan Y, Yan S, Wang Y (2014) Rapid detection of shrimp white spot syndrome virus by real time, isothermal recombinase polymerase amplification assay. PLOS One 9.

### Example 2—LAMP Assays for Shrimp Pathogens

This Example can demonstrate LAMP assays for five exemplary shrimp pathogens: White spot syndrome virus (WSSV), infectious hypodermal and haematopoietic necrosis virus (IHHNV), Hepatobacter penaei/Necrotising hepatopancreatitis (NHP), Vibrio spp.-causing Acute hepatopancreatic necrosis disease (AHPND) and Enterocytozoon hepatopenaei (EHP). This Example also demonstrates a penaeid shrimp internal control gene that would serve as a standard for the LAMP based-detection method. A limit of detection (LOD) for the assays have also been determined to be about 100 copies/reaction for at least WSSV, NHP and EHP. Advantages of the assays demonstrated here and elsewhere herein are at least that Infectious diseases can be detected at the pond site without the need to shipping samples in the laboratory located far away from the farms, and sometimes in altogether different geographic region, the workflow is easy to follow and perform, it does not require expensive or sensitive equipment (e.g., a thermocycler), faster than traditional approaches (e.g., conventional PCR), faster than traditional technology (the workflow demonstrated at least in this Example was completed in about 1 hour or less), results of the assay can be interpreted following simple instructions and does not require extensive expertise to complete, is cost-effective, and is adaptable so as to be able to be modified as needed by firms of limited means resources or infrastructure.

FIG. 4 shows a general workflow for a shrimp pathogen point of care assay described herein.

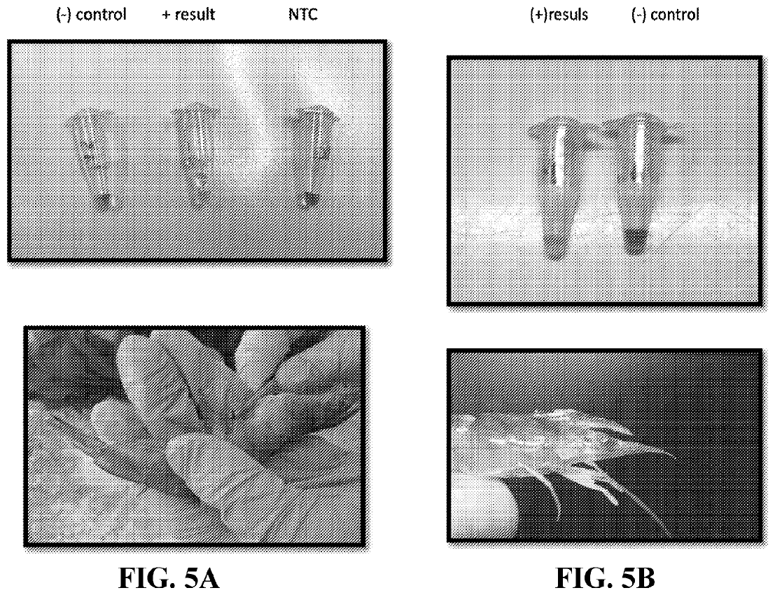

FIGS. 5A-5B show detection of (FIG. 5A) white spot syndrome virus (WSSV) and (FIG. 5B) infectious hypodermal and haematopoietic necrosis virus (IHHNV) and the clinical signs in shrimp of the respective diseases.

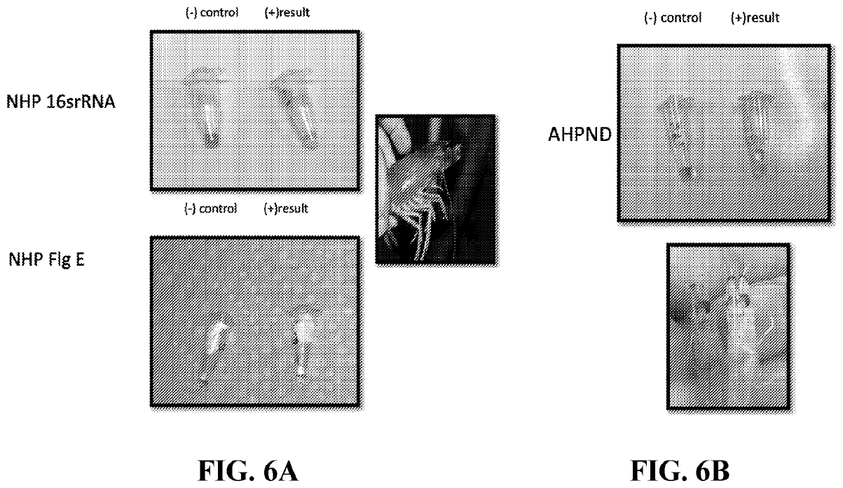

FIGS. 6A-6B show results from a LAMP isothermal method for shrimp bacterial pathogen detection. (FIG. 6A) Hepatobacter penaei/Necrotising hepatopancreatitis (NHP) detection targeting two different genes 16SrRNA & Flg E. (FIG. 6B) Vibrio parahaemolyticus causing-Acute hepatopancreatic necrosis disease (AHPND) detection targeting a toxin gene (Pir A). The clinical sign of shrimp affected by the corresponding diseases are shown alongside the pathogen detection panel by LAMP isothermal detection image.

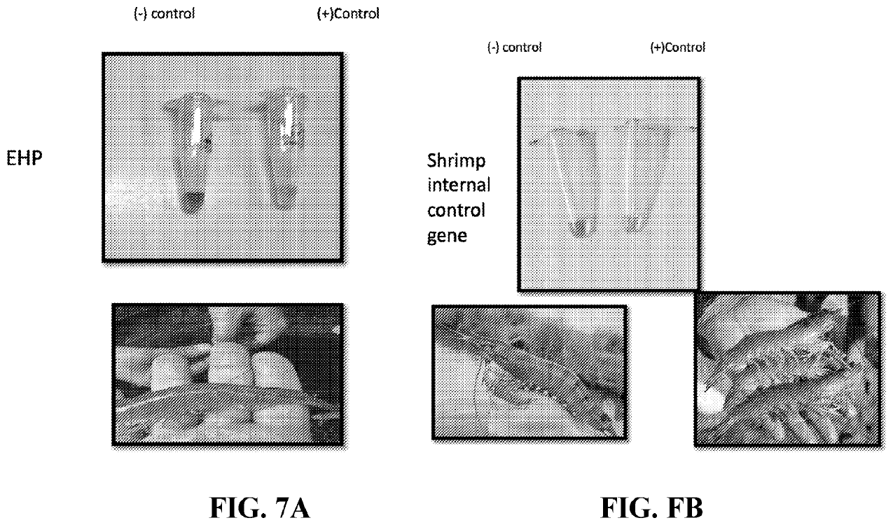

FIG. 7A-7B show results from a LAMP isothermal method for detection of (FIG. 7A) a microsporidium Enterocytozoon hepatopenaei (EHP) (FIG. 7B): an internal control gene of shrimp.

FIGS. 8A-8B show primers and a map of the regions of WSSV VP28 gene amplified by LAMP amplification (FIG. 8A) Primers sequences: (FIG. 8B): Genomic region of WSSV VP28 gene amplified. Dark green indicates the forward primers. Light green indicates the backward primers.

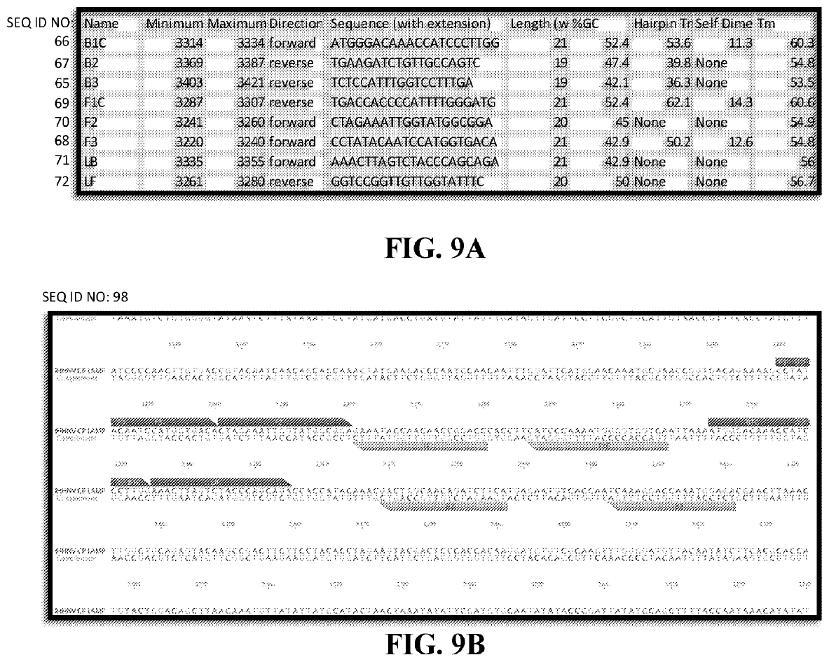

FIGS. 9A-9B show primers and a map of the regions of IHHNV CP gene targeted for amplification by LAMP amplification (FIG. 9A) Primers sequences: (FIG. 9B): Genomic region of IHHNV gene amplified. Dark green indicates the forward primers. Light green indicates the backward primers.

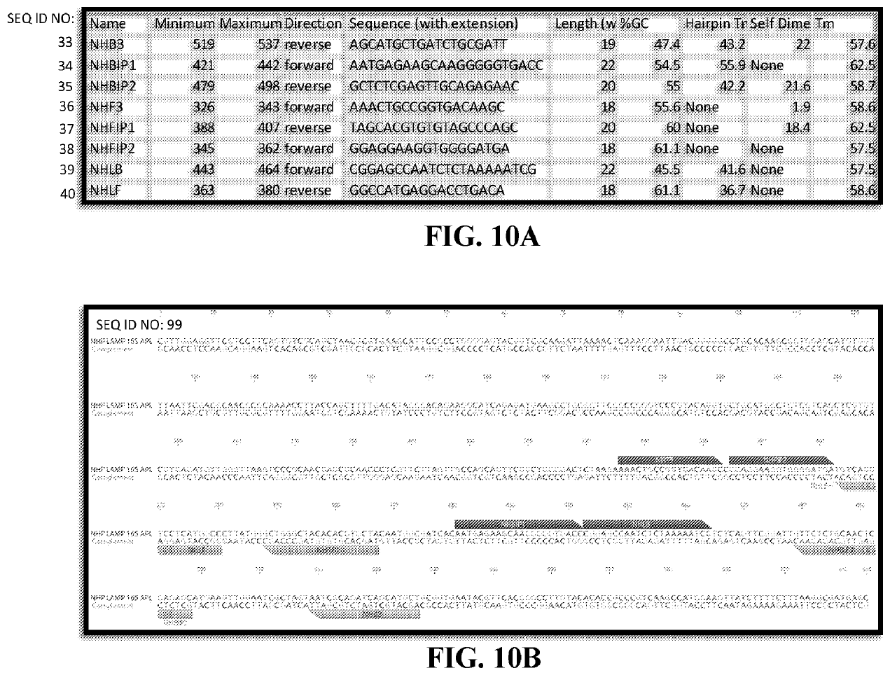

FIGS. 10A-10B show primers and a map of the regions of NHP 16S rRNA gene used for LAMP amplification (FIG. 10A) Primers sequences: (FIG. 10B): Genomic region of NHP 16S rRNA gene amplified. Dark green indicates the forward primers. Light green indicates the backward primers.

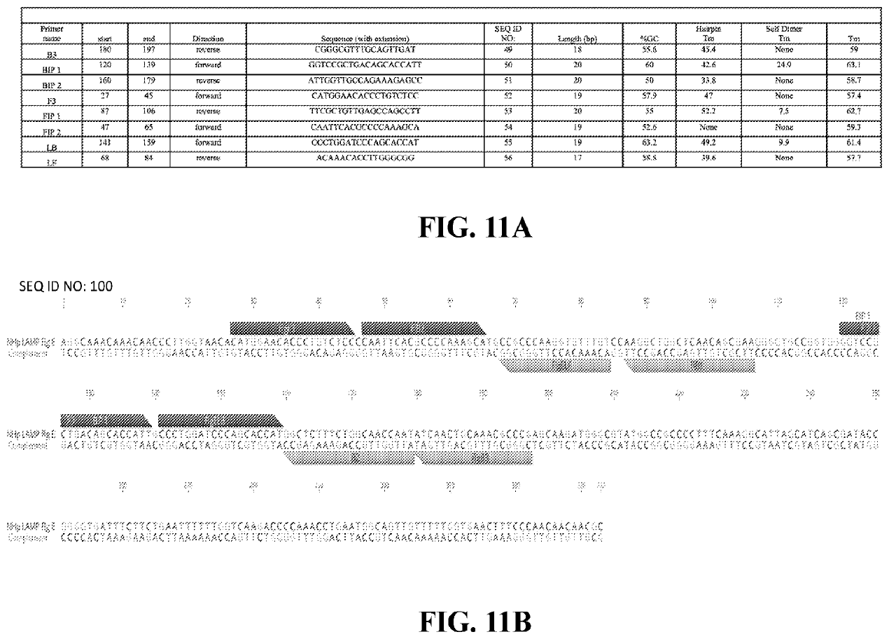

FIGS. 11A-11B show primers and a map of the regions of NHP Flg E gene used for LAMP amplification (FIG. 11A) Primers sequences: (FIG. 11B): Genomic region of NHP FlgE gene amplified. Dark green indicates the forward primers. Light green indicates the backward primers.

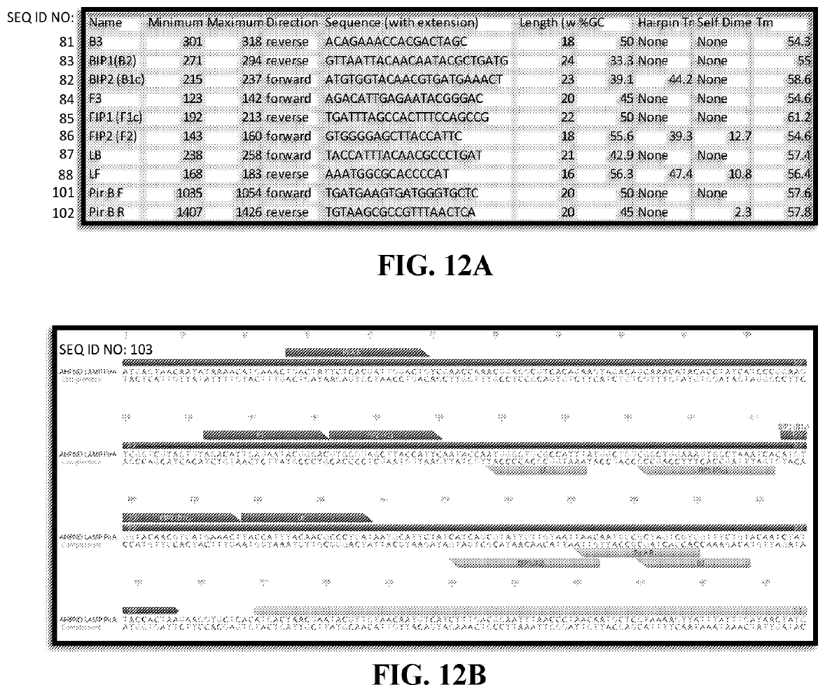

FIGS. 12A-12B show primers and a map of the regions of AHPND Pir A region used for LAMP amplification (FIG. 12A) Primers sequences: (FIG. 12B): Genomic region of AHPND PirA gene amplified. Dark green indicates the forward primers. Light green indicates the backward primers.

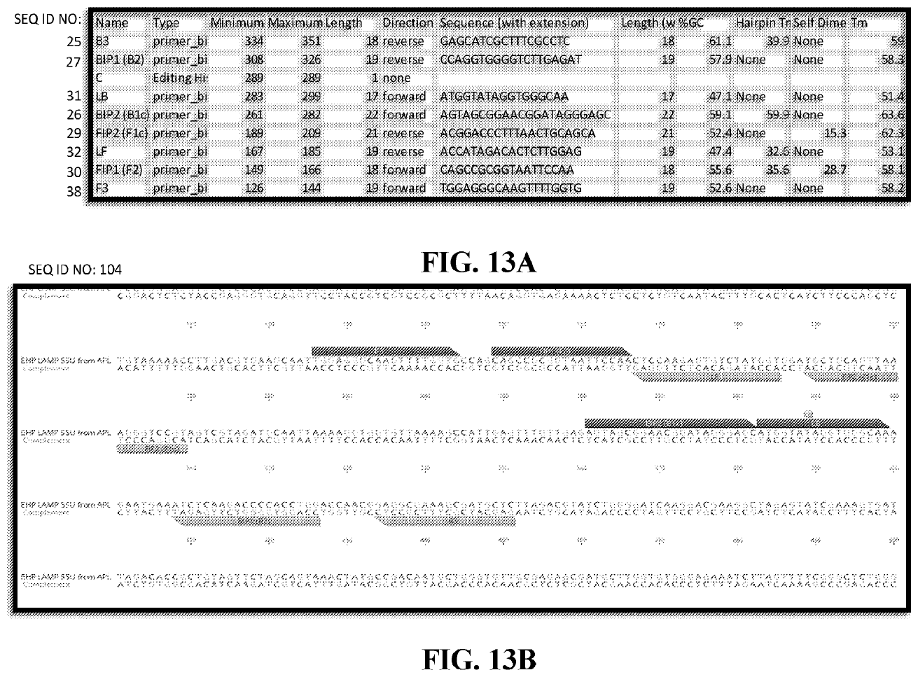

FIGS. 13A-13B show primers and a map of the regions of EHP SSU region used for LAMP amplification (FIG. 13A) Primers sequences: (FIG. 13B): Genomic region of EHP SSU region amplified. Dark green indicates the forward primers. Light green indicates the backward primers.

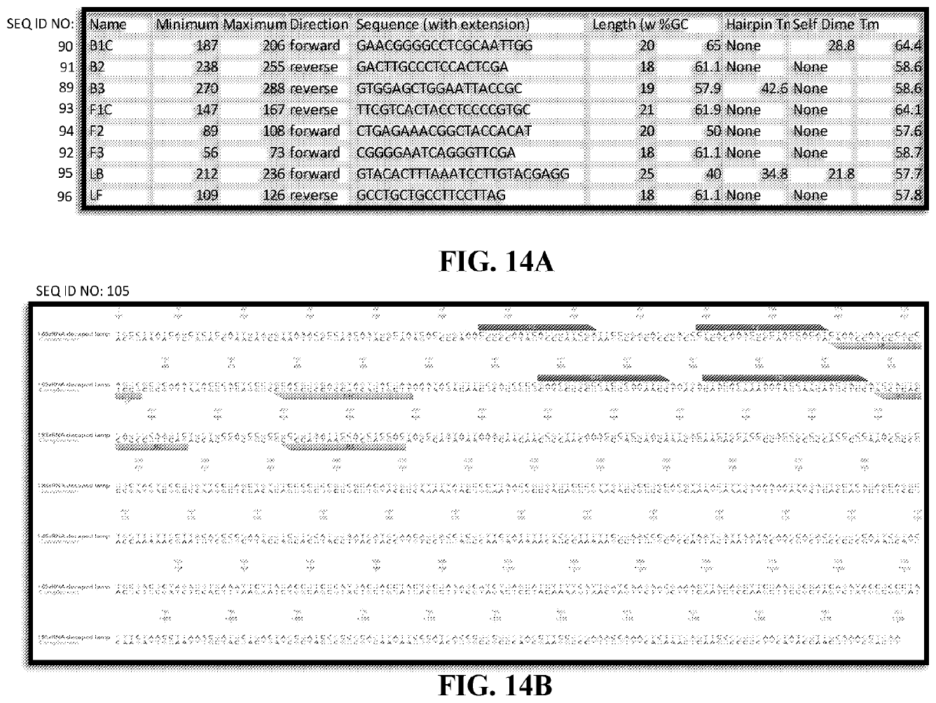

FIGS. 14A-14B show primers and a map of the regions of. Primers and Map of the regions of shrimp penaeid 18srRNA internal control used for LAMP amplification (A) Primers sequences: (FIG. 14B): Genomic region of penaeid shrimp 18S rRNA gene amplified. Dark green indicates the forward primers. Light green indicates the backward primers.

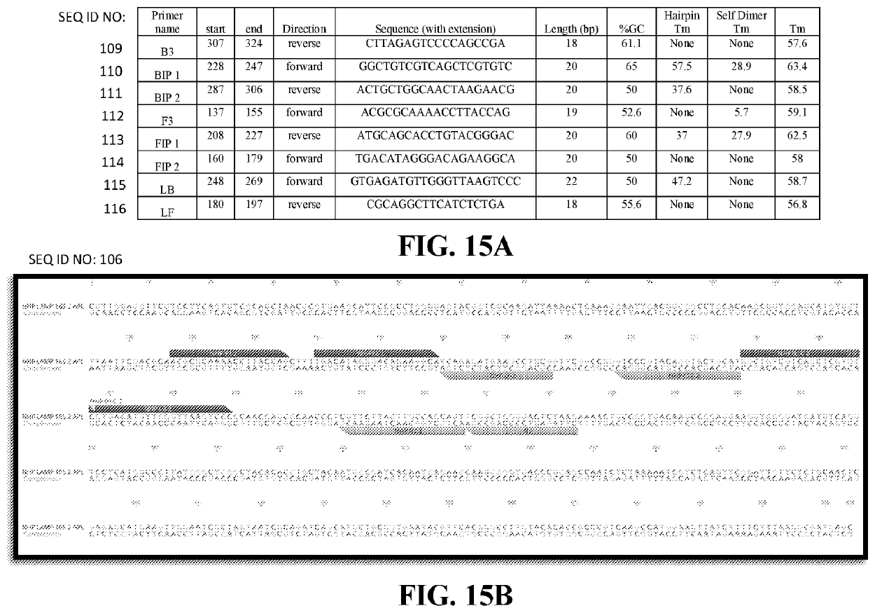

FIGS. 15A-15B show a table of primers (FIG. 15A) and a map (FIG. 15B) of the genomic region of NHP 16S rRNA 2 gene amplified. Dark green indicates the forward primers. Light green indicates the backward primers.

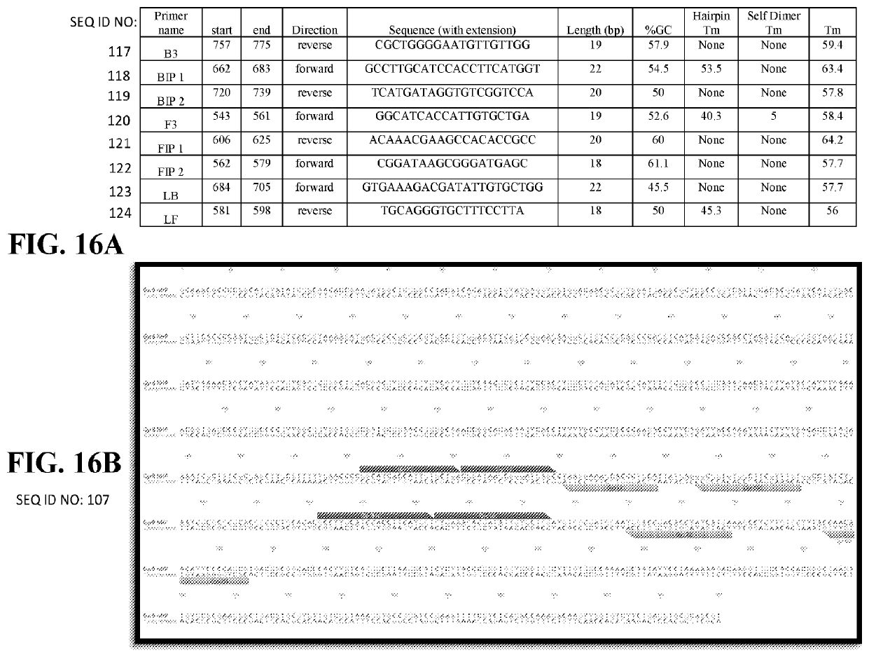

FIGS. 16A-16B show a table of primers (FIG. 16A) and a map (FIG. 16B) of the region of an NHP Gyr B. FIG. 16B shows the genomic region of NHP Gyr B gene amplified during a LAMP assay. Dark green indicates the forward primers. Light green indicates the backward primers.

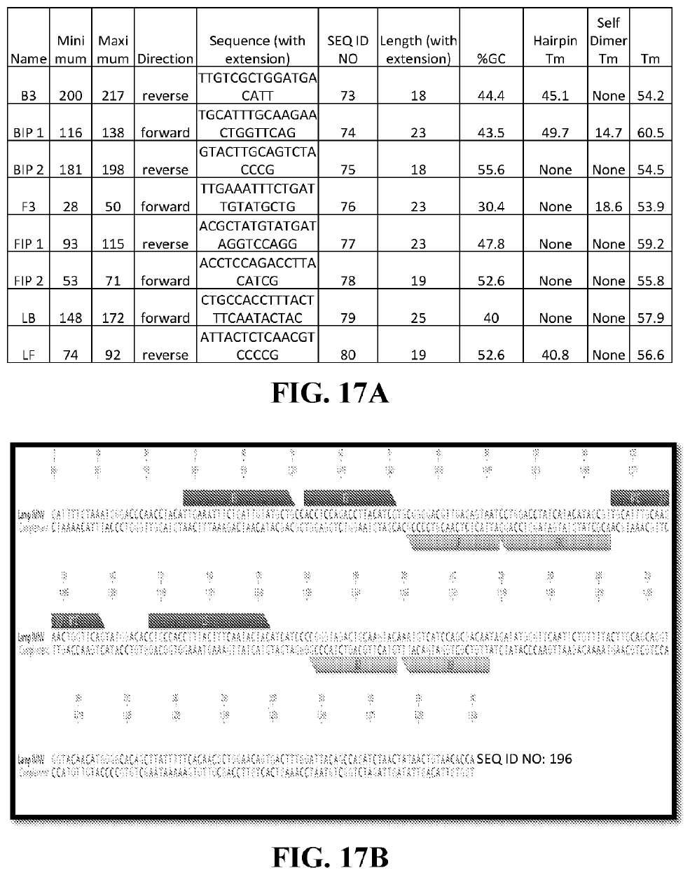

FIGS. 17A-17B show a table of primers (FIG. 17A) and a map (FIG. 17B) of the region of an ORF (ORF1, capsid protein) of infectious myconecrosis virus (IMNV) B. FIG. 17B shows the genomic region of IMNV ORF1 gene amplified during a LAMP assay. Dark green indicates the forward primers. Light green indicates the backward primers.

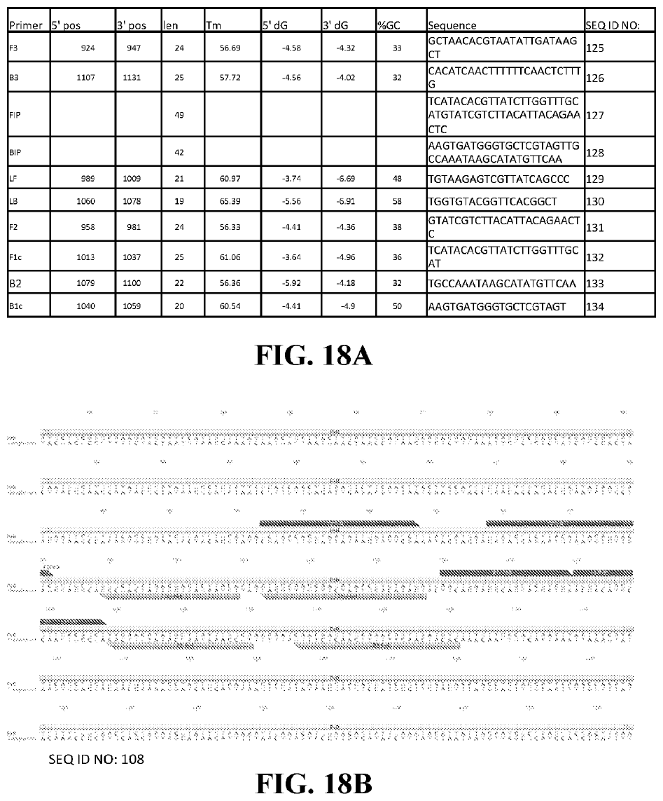

FIGS. 18A-18B show a table of primers (FIG. 18A) and a map (FIG. 18B) of the region of an AHPND pirB. FIG. 18B shows the genomic region of AHPND pirB gene amplified during a LAMP assay. Dark green indicates the forward primers. Light green indicates the backward primers.

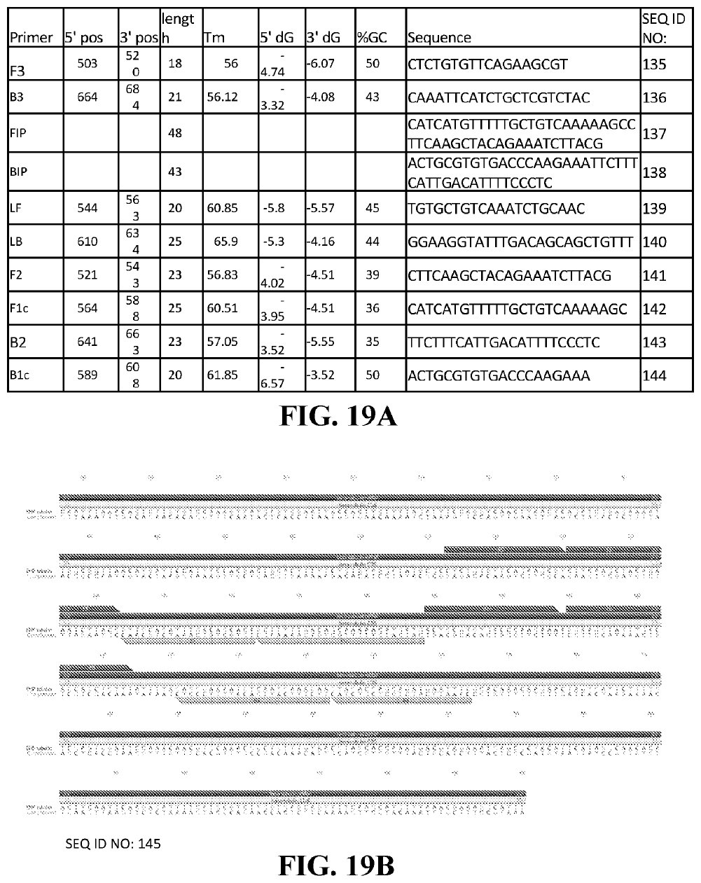

FIGS. 19A-19B show a table of primers (FIG. 19A) and a map (FIG. 19B) of the region of an EHP Beta tubulin. FIG. 19B shows the genomic region of EHP Beta tubulin gene amplified during a LAMP assay. Dark green indicates the forward primers. Light green indicates the backward primers.

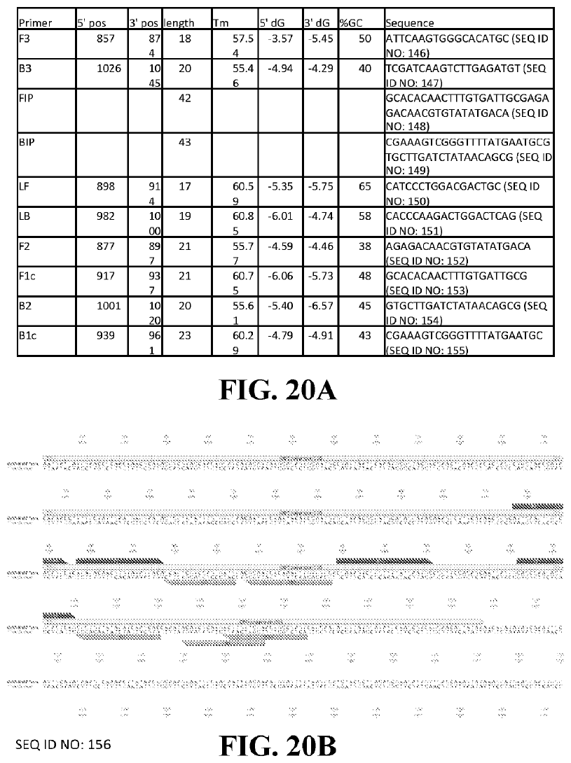

FIGS. 20A-20B show a table of primers (FIG. 20A) a map (FIG. 20B) of the region of an IMNV RdRp. FIG. 20B shows the genomic region of IMNV RdRp gene amplified during a LAMP assay. Dark green indicates the forward primers. Light green indicates the backward primers.

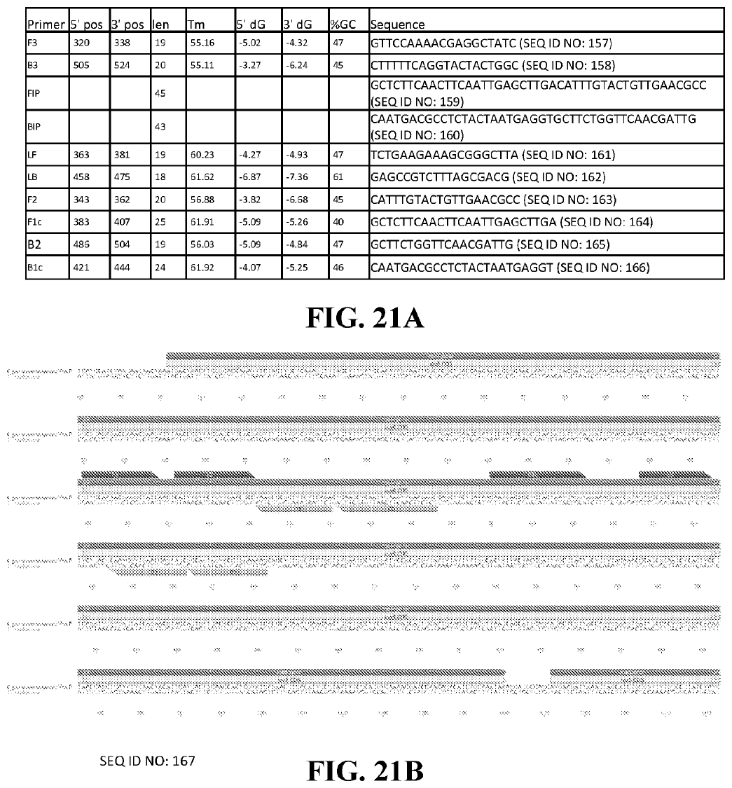

FIGS. 21A-21B show a table of primers (FIG. 21A) and a map (FIG. 21B) of the region of a Vibrio parahaemolyticus ToxR. FIG. 21B shows the genomic region of Vibrio parahaemolyticus ToxR gene amplified during a LAMP assay. Dark green indicates the forward primers. Light green indicates the backward primers.

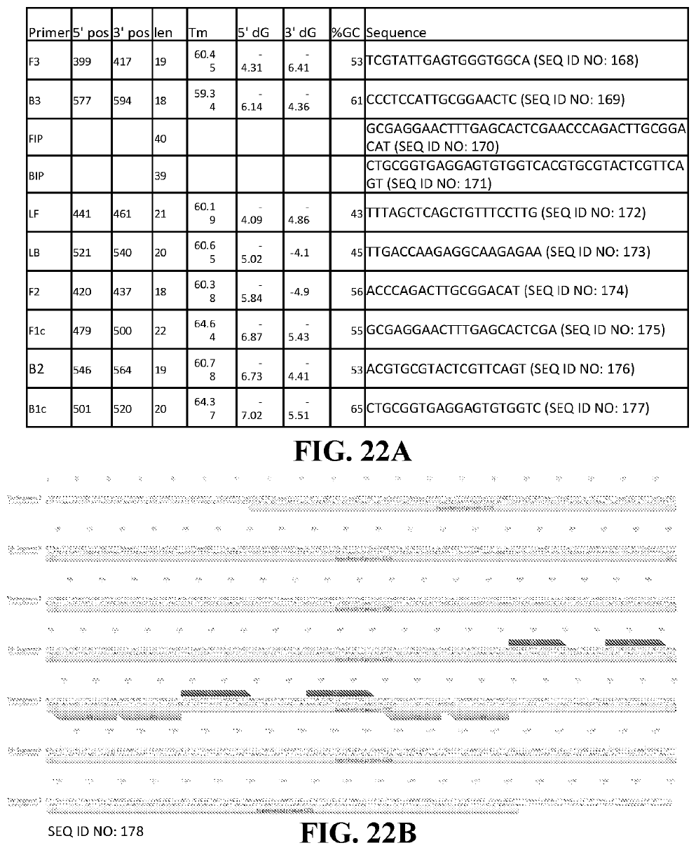

FIGS. 22A-22B show a table of primers (FIG. 22A) and a map (FIG. 22B) of the region of a Tilapia lake virus (TiLV) segment 3. FIG. 22B shows the genomic region of a Tilapia lake virus (TiLV) segment 3 gene amplified during a LAMP assay. Dark green indicates the forward primers. Light green indicates the backward primers.

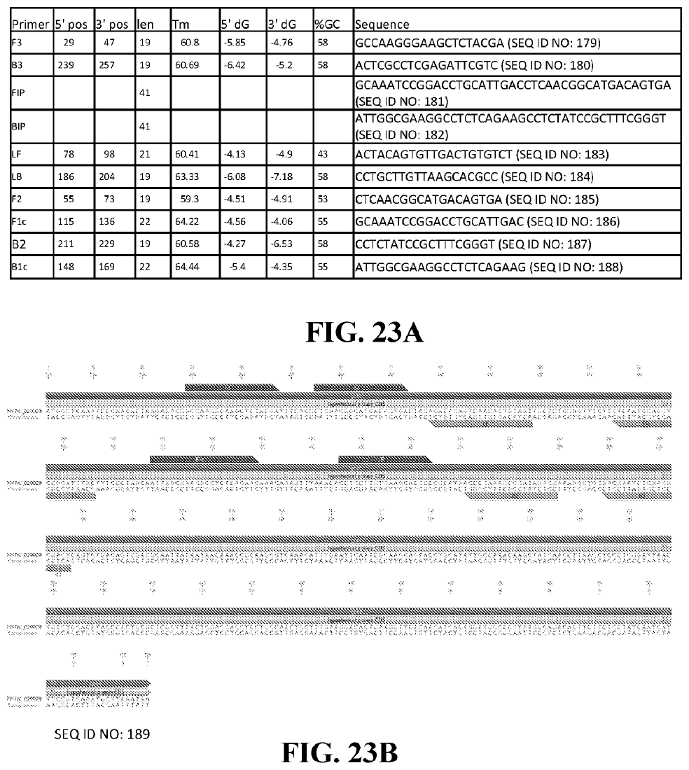

FIGS. 23A-23B show a table of primers (FIG. 23A) and a map (FIG. 23B) of the region of a Tilapia lake virus (TiLV) segment 8. FIG. 23B shows the genomic region of a Tilapia lake virus (TiLV) segment 8 gene amplified during a LAMP assay. Dark green indicates the forward primers. Light green indicates the backward primers.

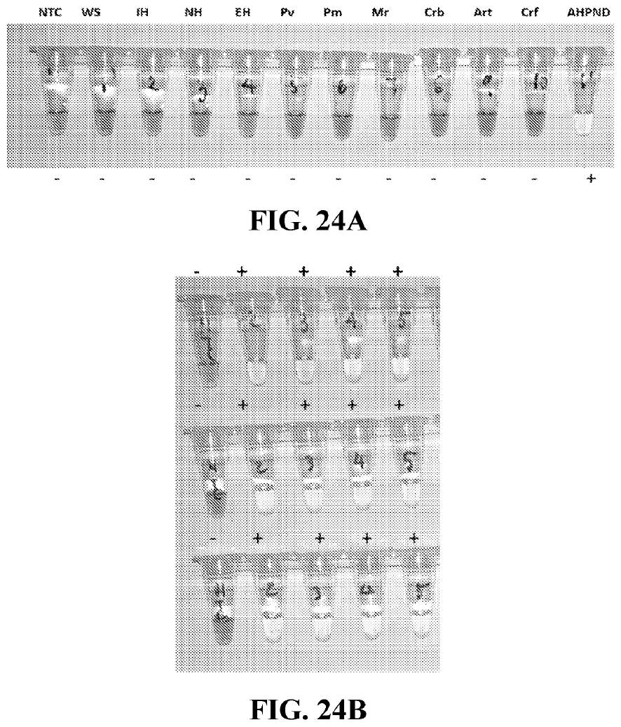

FIGS. 24A-24B show results demonstrating specificity (FIG. 24A) and sensitivity (FIG. 24B) in detecting AHPND targeting the pir B gene.

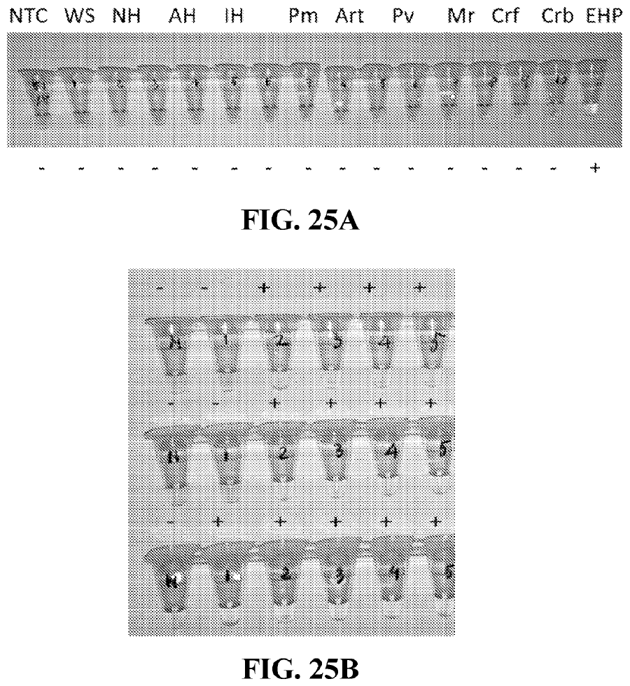

FIGS. 25A-25B show results demonstrating specificity (FIG. 25A) and sensitivity (FIG. 25B) in detecting EHP targeting the B-tubulin gene.

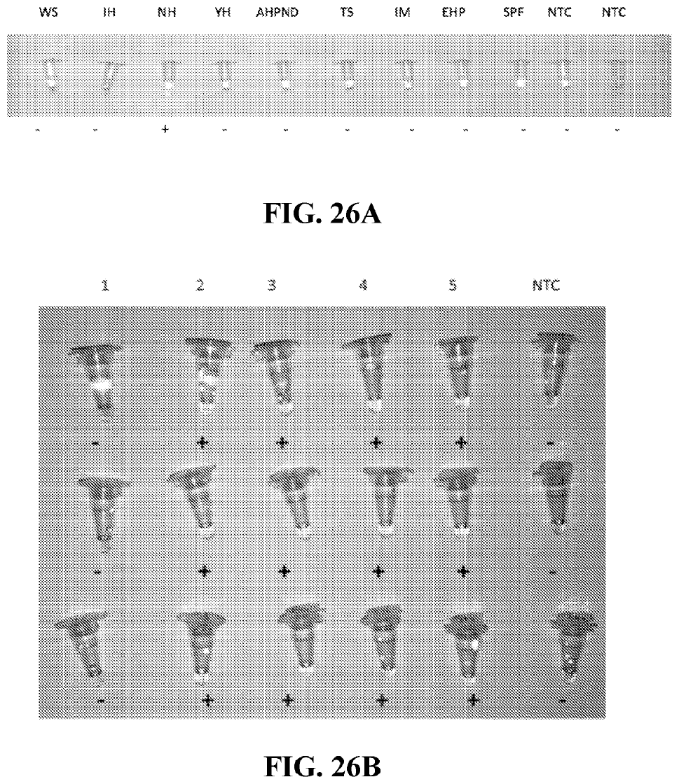

FIGS. 26A-26B show results demonstrating specificity (FIG. 26A) and sensitivity (FIG. 26B) in detecting NHP targeting the 16S rRNA2 gene.

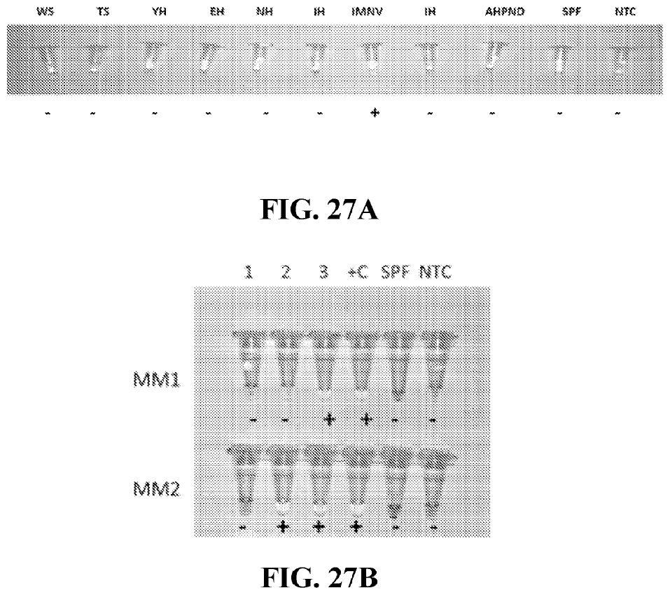

FIGS. 27A-27B show results demonstrating specificity (FIG. 27A) and sensitivity (FIG. 27B) in detecting IMNV targeting the capsid protein (CP) gene.

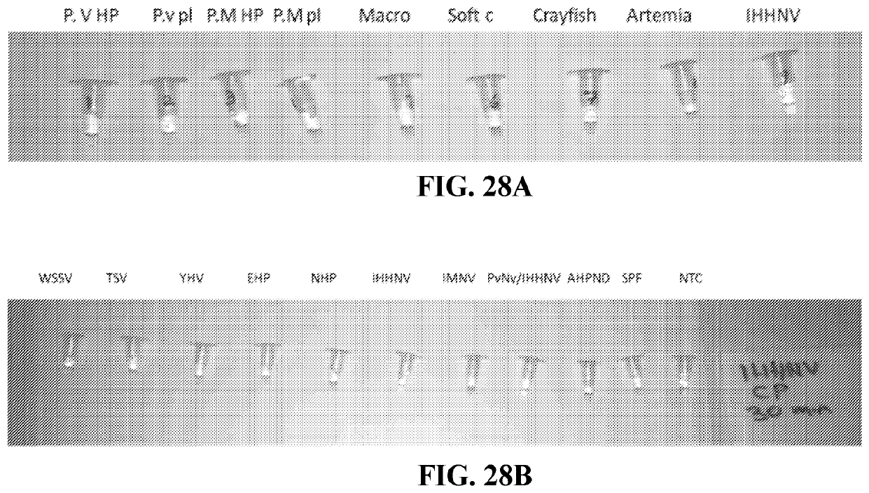

FIGS. 28A-28B show results demonstrating specificity (FIG. 28A) and sensitivity (FIG. 28B) in detecting IHHNV targeting the capsid protein (CP) gene.

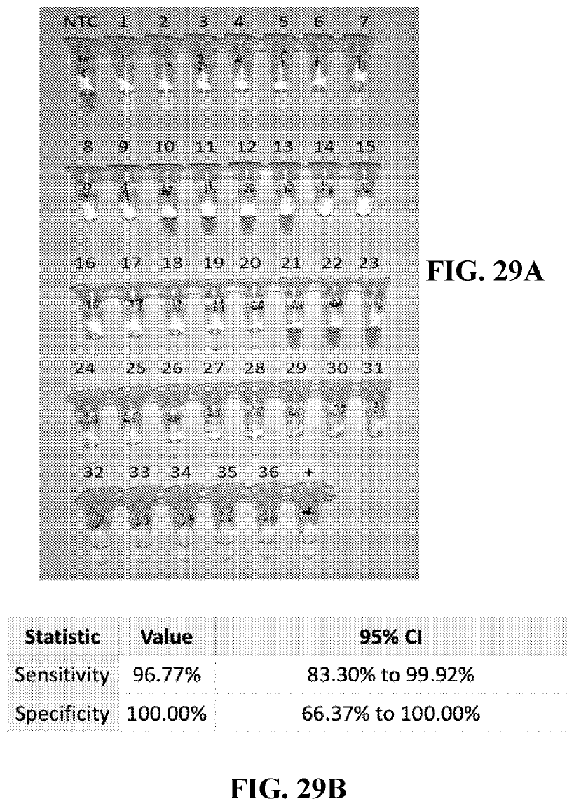

FIGS. 29A-29B show validation of AHPND detection targeting Pir B gene (FIG. 29A) and the sensitivity/specificity in detecting the AHPND Pir B gene (FIG. 29B).

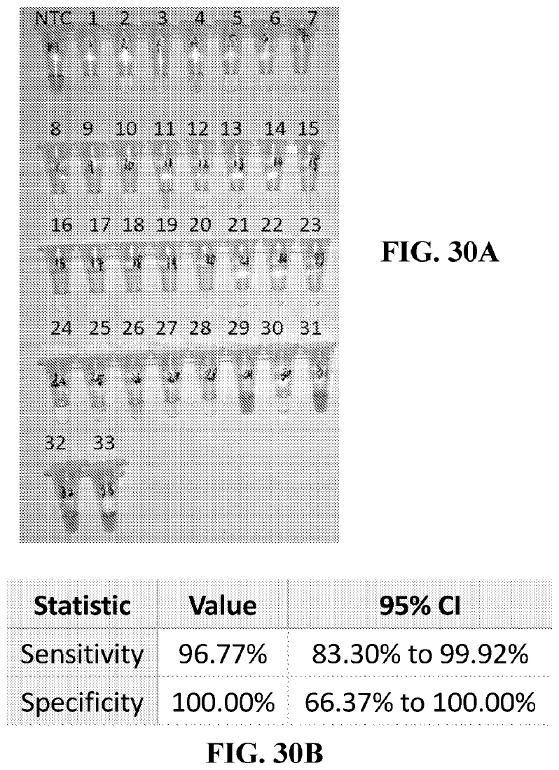

FIGS. 30A-30B show validation of EHP detection targeting beta tubulin gene (FIG. 30A) and the sensitivity/specificity in detecting the beta tubulin gene (FIG. 30B).

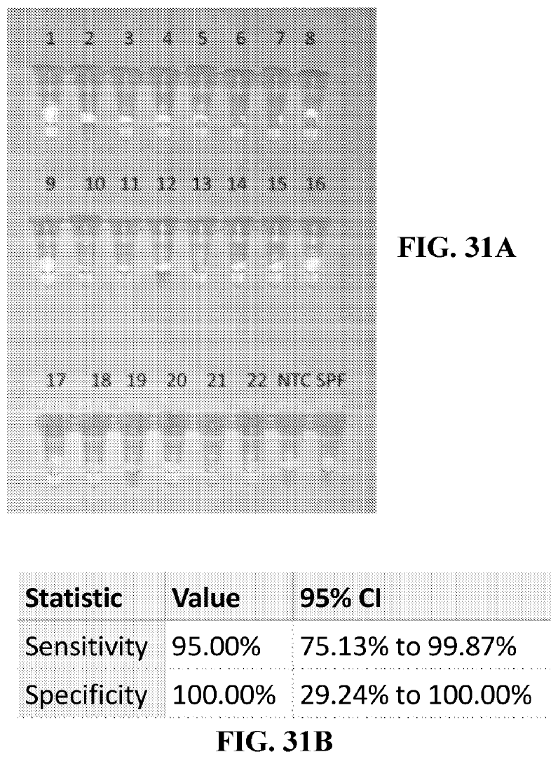

FIGS. 31A-31B show validation of NHP detection targeting beta tubulin gene (FIG. 31A) and the sensitivity/specificity in detecting the 16S rRNA 2 gene (FIG. 31B).

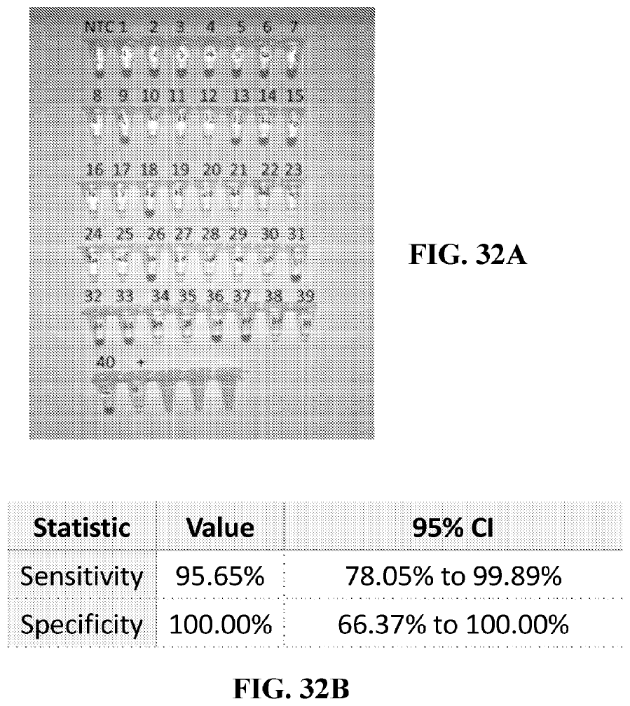

FIGS. 32A-32B show validation of IMNV detection targeting beta tubulin gene (FIG. 32A) and the sensitivity/specificity in detecting the capsid protein (CP) gene (FIG. 32B).

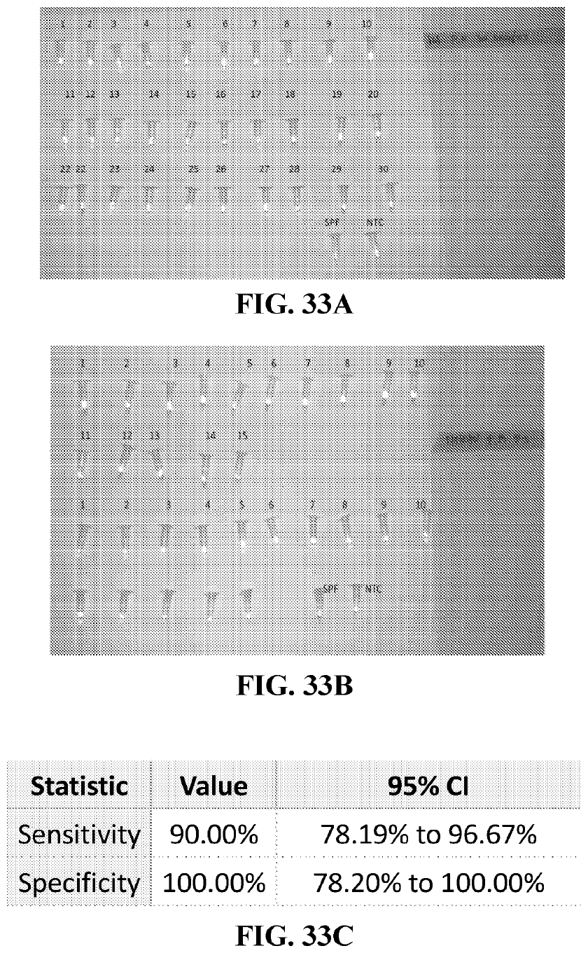

FIGS. 33A-33C show validation of IHHNV detection targeting capsid protein (CP) gene in P. vannamei (FIG. 33A) and P. monodon and P. stylirostyris (FIG. 33B) and the sensitivity/specificity in detecting IHHNV CP gene (FIG. 33C).

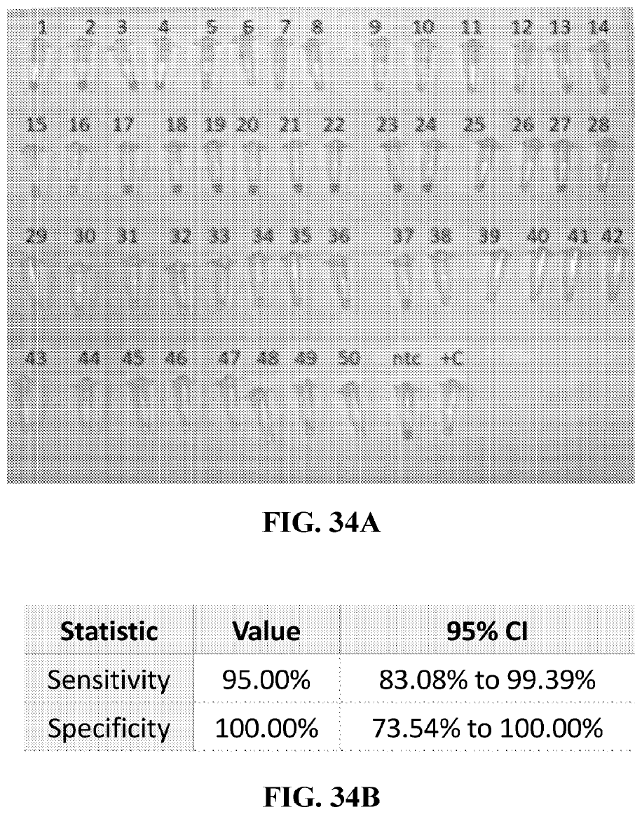

FIGS. 34A-34B show validation of WSSV detection (FIG. 34A) and the sensitivity/specificity in detecting WSSV (FIG. 34B).

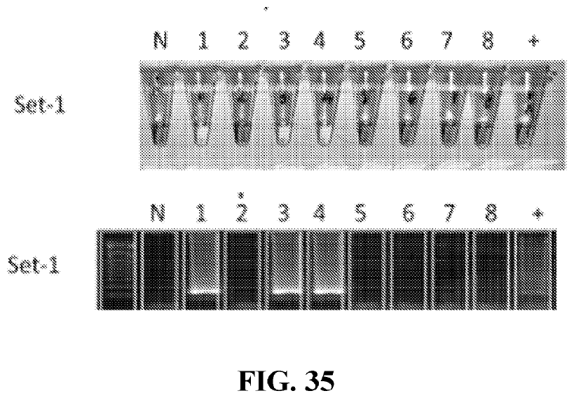

FIG. 35 show specificity in detecting Vibrio parahaemolyticus Tox R gene.

### Example 3—Exemplary Primers for Detection of Shrimp Pathogens

i. Proof of Concept for:
1. White Spot Syndrome Virus (WSSV) (shrimp)
2. Enterocytozoon hepatopenaei (EHP) (shrimp)
3. Necrotising Hepatopancreatitis/Hepatobacter penaei (NHP-B) (shrimp)
4. Infectious Hypodermal and Hematopoietic Necrosis Virus (IHHNV) (shrimp)
5. Infectious Myonecrosis Virus (IMNV) (shrimp)
6. Vibrio spp.-causing Acute Hepatopancreatic Necrosis Disease/Early Mortality Syndrome (AHPND/EMS) (shrimp)
7. Internal control Penaeus spp. 18SrRNA (shrimp)
8. Vibrio parahaemolyticus ToxR (shrimp)
9. Tilapia lake virus (TiLV) (tilapia)
   ii. Region and Primer Sets:
1. White Spot Syndrome Virus (WSSV)
   a. DNA target region: VP 28

| SEQ ID NO: 1 | |
|---|---|
| ATGGATCTTT CTTTCACTCT TTCGGTCGTG TCGGCCATCC TCGCCATCAC TGCTGTGATT | 60 |
| GCTGTATTTA TTGTGATTTT TAGGTATCAC AACACTGTGA CCAAGACCAT CGAAACCCAC | 120 |
| ACAGACAATA TCGAGACAAA CATGGATGAA AACCTCCGCA TTCCTGTGAC TGCTGAGGTT | 180 |
| GGATCAGGCT ACTTCAAGAT GACTGATGTG TCCTTTGACA GCGACACCTT GGGCAAAATC | 240 |
| AAGATCCGCA ATGGAAAGTC TGATGCACAG ATGAAGGAAG AAGATGCGGA TCTTGTCATC | 300 |
| ACTCCCGTGG AGGGCCGAGC ACTCGAAGTG ACTGTGGGGC AGAATCTCAC CTTTGAGGGA | 360 |
| ACATTCAAGG TGTGGAACAA CACATCAAGA AAGATCAACA TCACTGGTAT GCAGATGGTG | 420 |
| CCAAAGATTA ACCCATCAAA GGCCTTTGTC GGTAGCTCCA ACACCTCCTC CTTCACCCCC | 480 |
| GTCTCTATTG ATGAGGATGA AGTTGGCACC TTTGTGTGTG GTACCACCTT TGGCGCACCA | 540 |
| ATTGCAGCTA CCGCCGGTGG AAATCTTTTC GACATGTACG TGCACGTCAC CTACTCTGGC | 600 |
| ACTGAGACCG AGTAA | |

b. Primer region

TABLE 3

| | | | | | SEQ | | | | | |
|---|---|---|---|---|---|---|---|---|---|---|
| Primer | | | | Sequence | ID | Length | | Hairpin | Self Dimer | |
| name | start | end | Direction | (with extension) | NO: | (bp) | % GC | Tm | Tm | Tm |
| B3 | 590 | 609 | reverse | GGTCTCAGTGCCAGAGTAGG | 17 | 20 | 60 | 34.7 | None | 59.2 |
| BIP 1 | 523 | 541 | forward | ACCACCTTTGGCGCACCAA | 18 | 19 | 57.9 | 54.6 | 10.8 | 63 |
| BIP 2 | 568 | 586 | reverse | CGTGCACGTACATGTCGAT | 19 | 19 | 52.6 | 34.1 | 30.5 | 58.4 |
| F3 | 416 | 435 | forward | TGGTGCCAAAGATTAACCCA | 20 | 20 | 45 | None | None | 57.3 |
| FIP 1 | 501 | 522 | reverse | ACCACACACAAAGGTGCCAACT | 21 | 22 | 50 | 49.4 | None | 63. |
| FIP 2 | 444 | 463 | forward | CTTTGTCGGTAGCTCCAACA | 22 | 20 | 50 | 30.3 | None | 57.8 |
| LB | 546 | 562 | forward | AGCTACCGCCGGTGGAA | 23 | 17 | 64.7 | 36.2 | 40 | 60.7 |
| LF | 479 | 500 | reverse | TCATCCTCATCAATAGAGACGG | 24 | 22 | 45.5 | 35.1 | None | 56.9 |

c. Mapping of primers within the gene
   See e.g., FIG. 8B.
2. Enterocytozoon hepatopenaei (EHP)
   a. DNA target region: SSU (18srRNA)

| SEQ ID NO: 2 | |
|---|---|
| GCCTGAGAGA TGGCTCCCAC GTCCAAGGAT GGCAGCAGGC GCGAAAATTG TCCACTCTTT | 60 |
| TGAGAGGAGA CAGTTATGAA ACGTGAGTAG AAGGGTCGAG TGTAAAAACC TTGACGTGAA | 120 |
| GCAATTGGAG GGCAAGTTTT GGTGCCAGCA GCCGCGGTAA TTCCAACTCC AAGAGTGTCT | 180 |
| ATGGTGGATG CTGCAGTTAA AGGGTCCGTA GTCGTAGATG CAATTAAAAG GTGGTGTTAA | 240 |
| AAGCCATTGA GTTTGTTGAG AGTAGCGGAA CGGATAGGGA GCATGGTATA GGTGGGCAAA | 300 |
| GAATGAAATC TCAAGACCCC ACCTGGACCA ACGGAGGCGA AAGCGATGCT CTTAGACGTA | 360 |
| TCTGGGGATC AAGGACGAAG GCTAGAGTAT CGAAAGTGAT TAGACACCGC TGTAGTTCTA | 420 |
| GCAGTAAACT ATGCCGACAA TGCTGGGTGT TGCGAGAGCG ATGCTTGGTG TGGGAGAAAT | 480 |
| CTTAGTTTTC GGGCTCTGGG GATAGTACGC | |

b. Primer region

TABLE 4

| | | | | | | | | | Self | |
|---|---|---|---|---|---|---|---|---|---|---|
| Primer | | | | | SEQ | Length | | Hairpin | Dimer | |
| name | start | end | Direction | Sequence (with extension) | ID NO: | (bp) | % GC | Tm | Tm | Tm |
| B3 | 334 | 351 | reverse | GAGCATCGCTTTCGCCTC | 25 | 18 | 61.1 | 39.9 | None | 59 |
| BIP 1 | 261 | 282 | forward | AGTAGCGGAACGGATAGGG | 26 | 22 | 59.1 | 59.9 | None | 63.6 |
| | | | | AGC | | | | | | |
| BIP 2 | 308 | 326 | reverse | CCAGGTGGGGTCTTGAGAT | 27 | 19 | 57.9 | None | None | 58.3 |
| F3 | 126 | 144 | forward | TGGAGGGCAAGTTTTGGTG | 28 | 19 | 52.6 | None | None | 58.2 |
| FIP 1 | 189 | 209 | reverse | ACGGACCCTTTAACTGCAGC | 29 | 21 | 52.4 | None | 15.3 | 62.3 |
| | | | | A | | | | | | |
| FIP 2 | 149 | 166 | forward | CAGCCGCGGTAATTCCAA | 30 | 18 | 55.6 | 35.6 | 28.7 | 58.1 |
| LB | 283 | 299 | forward | ATGGTATAGGTGGGCAA | 31 | 17 | 47.1 | None | None | 51.4 |
| LF | 167 | 185 | reverse | ACCATAGACACTCTTGGAG | 32 | 19 | 47.4 | 32.6 | None | 53.1 |

c. Mapping of primers within the gene
See e.g., FIG. 13B.
3. Necrotising Hepatopancreatitis/Hepatobacter penaei (NHP-B)
   a. DNA target region: 16SrRNA, Flg E, Gyr B
   b. Primer regions

| 16SrRNA | |
|---|---|
| SEQ ID NO: 3 | |
| CGTTGGAGGT TCGTCCTTCA GTGTCGCAGC TAACGCGTGA AGCATTCCGC CTGGGGAGTA | 60 |
| CGGTCGCAAG ATTAAAACTC AAAGGAATTG ACGGGGGCCT GCACAAGCGG TGGAGCATGT | 120 |
| GGTTTAATTC GACGCAACGC GCAAAACCTT ACCAGCTTTT GACATAGGGA CAGAAGGCAT | 180 |
| CAGAGATGAA GCCTGCGGTT CGGCCGGGTC CCGTACAGGT GCTGCATGGC TGTCGTCAGC | 240 |
| TCGTGTCGTG AGATGTTGGG TTAAGTCCCG CAACGAGCGC AACCCTCGTT CTTAGTTGCC | 300 |
| AGCAGTTCGG CTGGGGACTC TAAGAAAACT GCCGGTGACA AGCCGGAGGA AGGTGGGGAT | 360 |
| GATGTCAGGT CCTCATGGCC CTTATGGGCT GGGCTACACA CGTGCTACAA TGGCGATCAC | 420 |
| AATGAGAAGC AAGGGGGTGA CCCGGAGCCA ATCTCTAAAA ATCGTCTCAG TTCGGATTGT | 480 |
| TCTCTGCAAC TCGAGAGCAT GAAGTTGGAA TCGCTAGTAA TCGCAGATCA GCATGCTGCG | 540 |
| GTGAATACGT TCACGGGCCT TGTACACACC GCCCGTCAAG CCATGGAAGT TATCTTTTCT | 600 |
| TTAAGGCGAT GAGC | |
| Flg E | |
| SEQ ID NO: 4 | |
| AGGCAAACAA ACAACCCTTG GTAACACATG GAACACCCTG TCTCCCCAAT TCACGCCCCA | 60 |
| AAGCATGCCG CCCAAGGTGT TTGTCCAAGG CTGGCTCAAC AGCGAAGGGG TGCCGGTGGG | 120 |
| GTCCGCTGAC AGCACCATTG CCCTGGATCC CAGCACCATG GCTCTTTCTG GCAACCAATA | 180 |
| TCAACTGCAA ACGCCCGAGC AAGATGGGCG TATGGCCGCC CCTTTCAAAG GCATTAGCAT | 240 |
| CAGCGATACC GGGGTGATTT CTTCTGAATT TTTTGGTCAA GACCCCAAAC CTGAATGGCA | 300 |
| GTTGTTTTTG GTGAACTTTC CCAACAACAA CGC | 333 |
| Gyr B | |
| SEQ ID NO: 5 | |
| GCAAACGCCC CGGCATGTAT ATCGGCAACA CCGAAGATGG CTCGGGCCTA CATCACATGG | 60 |
| TGTATGAGGT GGTGGACAAC GCGGTGGATG AGGCGCTGGC CGGTCATTGC ACACGCATTC | 120 |
| ATGTGACGCT TCACCCCAAT GGCTCTGTCA CGGTAAAAGA TGACGGCCGC GGCGTGCCTG | 180 |
| TGGACATTCA TGCGGAAGAA GGCGTGTCCG CAGCCCAGGT CATCATGACC CACCTACATG | 240 |
| CAGGGGGTAA GTTTGATGAA AACAGCTATA AGGTGTCAGG CGGGCTGCAC GGCGTGGGTG | 300 |
| TGTCGGTGGT CAACGCGCTG TCCACGTGGC TGGAGATGCG CATTTGGCGG GCCGGCAAAG | 360 |
| AACATGTTAT ACGATTTGAA AACGGTGAGC CCGTGGCCCC GTTGGCGGTG GTGGGCGATG | 420 |
| CGGAACCGGG GCAAACCGGC ACACAAGTCA CCTTTTTGCC TTCGCCCAAG ACCTTTCAGA | 480 |
| TTACCAAATT TTCTTTTGAC ACCTTAGAAA ATCGCTTTCG CGAGCTTGCG TTTTTGAACC | 540 |
| CCGGCATCAC CATTGTGCTG ACGGATAAGC GGGATGAGCC TAAGGAAAGC ACCCTGCATT | 600 |
| ATGAGGGCGG TGTGGCTTCG TTTGTGGCCT ATCTGAATCG CGCGCGCACG GCCCTGCACA | 660 |
| CGCCTTGCAT CCACCTTCAT GGTGTGAAAG ACGATATTGT GCTGGATGTG GCGATGGAAT | 720 |
| GGACCGACAC CTATCATGAA AGCCTCTTGT GCTTTACCAA CAACATTCCC CAGCGTGACG | 780 |
| GCGGCACCCA CTTGTCAGGG CTTAAGGCAT CTCTTACCCG GGTGGTGGGT GGCTTTGTGG | 840 |
| CCAAAAGCAA TATTCCTAAA AAAGACAAGG TGGCCACCAC GGGCGAAGAT GTGCGCGAAG | 900 |
| GGCTGACGTG CGTGTTGTCG GTAAAGGTGC CGGATCCGAA ATTTTCGTCT CAGACCAAAG | 960 |
| ACAAGTTGGT GTCTTCTGAG GTGCGCAGCG T | 991 |

### 16SrRNA 1

TABLE 5

| | | | | | SEQ | | | | Self | |
|---|---|---|---|---|---|---|---|---|---|---|
| Primer | | | | | ID | Length | | Hairpin | Dimer | |
| name | start | end | Direction | Sequence (with extension) | NO: | (bp) | % GC | Tm | Tm | Tm |
| B3 | 519 | 537 | reverse | AGCATGCTGATCTGCGATT | 33 | 19 | 47.4 | 43.2 | 22 | 57.6 |
| BIP 1 | 421 | 442 | forward | AATGAGAAGCAAGGGGGTG | 34 | 22 | 54.5 | 55.9 | None | 62.5 |
| | | | | ACC | | | | | | |
| BIP 2 | 479 | 498 | reverse | GCTCTCGAGTTGCAGAGAAC | 35 | 20 | 55 | 42.2 | 21.6 | 58.7 |
| F3 | 326 | 343 | forward | AAACTGCCGGTGACAAGC | 36 | 18 | 55.6 | None | 1.9 | 58.6 |
| FIP 1 | 388 | 407 | reverse | TAGCACGTGTGTAGCCCAGC | 37 | 20 | 60 | None | 18.4 | 62.5 |
| FIP 2 | 345 | 362 | forward | GGAGGAAGGTGGGGATGA | 38 | 18 | 61.1 | None | None | 57.5 |
| LB | 443 | 464 | forward | CGGAGCCAATCTCTAAAAAT | 39 | 22 | 45.5 | 41.6 | None | 57.5 |
| | | | | CG | | | | | | |
| LF | 363 | 380 | reverse | GGCCATGAGGACCTGACA | 40 | 18 | 61.1 | 36.7 | None | 58.6 |

### 16SrRNA 2

TABLE 6

| | | | | | SEQ | | | | | |
|---|---|---|---|---|---|---|---|---|---|---|
| Primer | | | | | ID | Length | | Hairpin | Self Dimer | |
| name | start | end | Direction | Sequence (with extension) | NO: | (bp) | % GC | Tm | Tm | Tm |
| B3 | 307 | 324 | reverse | CTTAGAGTCCCCAGCCGA | 41 | 18 | 61.1 | None | None | 57.6 |
| BIP 1 | 228 | 247 | forward | GGCTGTCGTCAGCTCGTGTC | 42 | 20 | 65 | 57.5 | 28.9 | 63.4 |
| BIP 2 | 287 | 306 | reverse | ACTGCTGGCAACTAAGAACG | 43 | 20 | 50 | 37.6 | None | 58.5 |
| F3 | 137 | 155 | forward | ACGCGCAAAACCTTACCAG | 44 | 19 | 52.6 | None | 5.7 | 59.1 |
| FIP 1 | 208 | 227 | reverse | ATGCAGCACCTGTACGGGAC | 45 | 20 | 60 | 37 | 27.9 | 62.5 |
| FIP 2 | 160 | 179 | forward | TGACATAGGGACAGAAGGCA | 46 | 20 | 50 | None | None | 58 |
| LB | 248 | 269 | forward | GTGAGATGTTGGGTTAAGTCCC | 47 | 22 | 50 | 47.2 | None | 58.7 |
| LF | 180 | 197 | reverse | CGCAGGCTTCATCTCTGA | 48 | 18 | 55.6 | None | None | 56.8 |

Flg E

TABLE 7

| | | | | | SEQ | | | | | |
|---|---|---|---|---|---|---|---|---|---|---|
| Primer | | | | | ID | Length | | Hairpin | Self Dimer | |
| name | start | end | Direction | Sequence (with extension) | NO | (bp) | % GC | Tm | Tm | Tm |
| B3 | 180 | 197 | reverse | CGGGCGTTTGCAGTTGAT | 49 | 18 | 55.6 | 45.4 | None | 59 |
| BIP 1 | 120 | 139 | forward | GGTCCGCTGACAGCACCATT | 50 | 20 | 60 | 42.6 | 24.9 | 63.1 |
| BIP 2 | 160 | 179 | reverse | ATTGGTTGCCAGAAAGAGCC | 51 | 20 | 50 | 33.8 | None | 58.7 |
| F3 | 27 | 45 | forward | CATGGAACACCCTGTCTCC | 52 | 19 | 57.9 | 47 | None | 57.4 |
| FIP 1 | 87 | 106 | reverse | TTCGCTGTTGAGCCAGCCTT | 53 | 20 | 55 | 52.2 | 7.5 | 62.7 |
| FIP 2 | 47 | 65 | forward | CAATTCACGCCCCAAAGCA | 54 | 19 | 52.6 | None | None | 59.3 |
| LB | 141 | 159 | forward | CCCTGGATCCCAGCACCAT | 55 | 19 | 63.2 | 49.2 | 9.9 | 61.4 |
| LF | 68 | 84 | reverse | ACAAACACCTTGGGCGG | 56 | 17 | 58.8 | 39.6 | None | 57.7 |

Gyr B

TABLE 8

| | | | | | SEQ | | | | | |
|---|---|---|---|---|---|---|---|---|---|---|
| Primer | | | | | ID | Length | | Hairpin | Self Dimer | |
| name | start | end | Direction | Sequence (with extension) | NO: | (bp) | % GC | Tm | Tm | Tm |
| B3 | 757 | 775 | reverse | CGCTGGGGAATGTTGTTGG | 57 | 19 | 57.9 | None | None | 59.4 |
| BIP 1 | 662 | 683 | forward | GCCTTGCATCCACCTTCATGGT | 58 | 22 | 54.5 | 53.5 | None | 63.4 |
| BIP 2 | 720 | 739 | reverse | TCATGATAGGTGTCGGTCCA | 59 | 20 | 50 | None | None | 57.8 |
| F3 | 543 | 561 | forward | GGCATCACCATTGTGCTGA | 60 | 19 | 52.6 | 40.3 | 5 | 58.4 |
| FIP 1 | 606 | 625 | reverse | ACAAACGAAGCCACACCGCC | 61 | 20 | 60 | None | None | 64.2 |
| FIP 2 | 562 | 579 | forward | CGGATAAGCGGGATGAGC | 62 | 18 | 61.1 | None | None | 57.7 |
| LB | 684 | 705 | forward | GTGAAAGACGATATTGTGCTGG | 63 | 22 | 45.5 | None | None | 57.7 |
| LF | 581 | 598 | reverse | TGCAGGGTGCTTTCCTTA | 64 | 18 | 50 | 45.3 | None | 56 |

c. Mapping of primers within the gene
16SrRNA 1
See e.g., FIG. 10B.
16SrRNA 2
See e.g., FIG. 15B.
Flg E
See e.g., FIG. 11B.
Gyr B
See e.g., FIG. 16B.
4. Infectious Hypodermal and Hematopoietic Necrosis Virus (IHHNV)
   a. DNA target region: Capsid protein

| SEQ ID NO: 6 | |
|---|---|
| CATTTGAGAC TCTCACATTT ACAGACACCC CATATTTAGA AATATTTAAG GATACTACTG | 3060 |
| GACTACATAA TCAACTATCA ACTAAGGAAG CCGACGTAAC ATTGGCAAAG TGGATACAAA | 3120 |
| ATCCCCAACT TGTGACCGTA CAATCAACAG CAGCAAACTA TGAAGACCCA ATCCAACAAT | 3180 |
| TTGGATTCAT GGAACAAATG CGAACCGGTG ACAGAAAAGC CTATACAATC CATGGTGACA | 3240 |
| CTAGAAATTG GTATGGCGGA GAAATACCAA CAACCGGACC CACCTTCATC CCAAAATGGG | 3300 |
| GTGGTCAATT AAAATGGGAC AAACCATCCC TTGGAAACTT AGTCTACCCA GCAGACCACC | 3360 |
| ATACAAACGA CTGGCAACAG ATCTTCATGA GAATGTCACC AATCAAAGGA CCAAATGGAG | 3420 |
| ACGAACTTAA ACTTGGCTGC AGAGTACAAG CCGACTTCTT CCTACACCTA GAAGTACGAC | 3480 |
| TCCCACCACA AGGATGTGTC GCAAGTTTGG GGATGTTACA ATATCTTCAC GCACCATGTA | 3540 |
| CTGGACAGCT TAACAAATGT TATATTATGC ATACTAACTA AATATATTCG ATGTGCAATA | 3600 |
| TATACCCGAT TATATCCAGC TTTTACCAAT AAACATATAT AGCTTATTAT CATCTATACC | 3660 |
| TACCCTATAT ACATACCAGC TACCCAGGCA AGGTGGGACT CCGGCTACCC AGGCATGGTG | 3720 |
| GGACACTTTT CTTCTATTGA CGACATATTT CGTCACTTAC GTCACTTACA AAAGACTAAA | 3780 |
| ATCCCTATCG TCAGTCAGTC ATTTAGAGTC AGGGATATTG TCCGCCGTCA CTTAGAGCGC | 3840 |
| GAAGCGCGAG TATCCATCAT TTAAATTAGT GGTATGACGT CACATATTAA GTTAACGGTT | 3900 |

b. Primer regions

TABLE 9

| | | | | | SEQ | | | | | |
|---|---|---|---|---|---|---|---|---|---|---|
| Primer | | | | | ID | Length | | Hairpin | Self Dimer | |
| name | start | end | Direction | Sequence (with extension) | NO: | (bp) | % GC | Tm | Tm | Tm |
| B3 | 3403 | 3421 | reverse | TCTCCATTTGGTCCTTTGA | 65 | 19 | 42.1 | 36.3 | None | 53.5 |
| BIP 1 | 3314 | 3334 | forward | ATGGGACAAACCATCCCTTGG | 66 | 21 | 52.4 | 53.6 | 11.3 | 60.3 |
| BIP 2 | 3369 | 3387 | reverse | TGAAGATCTGTTGCCAGTC | 67 | 19 | 47.4 | 39.8 | None | 54.8 |
| F3 | 3220 | 3240 | forward | CCTATACAATCCATGGTGACA | 68 | 21 | 42.9 | 50.2 | 12.6 | 54.8 |
| FIP 1 | 3287 | 3307 | reverse | TGACCACCCCATTTTGGGATG | 69 | 21 | 52.4 | 62.1 | 14.3 | 60.6 |
| FIP 2 | 3241 | 3260 | forward | CTAGAAATTGGTATGGCGGA | 70 | 20 | 45 | None | None | 54.9 |
| LB | 3335 | 3355 | forward | AAACTTAGTCTACCCAGCAGA | 71 | 21 | 42.9 | None | None | 56 |
| LF | 3261 | 3280 | reverse | GGTCCGGTTGTTGGTATTTC | 72 | 20 | 50 | None | None | 56.7 |

c. Mapping of primers within the gene
See e.g., FIG. 9B.
5. Infectious Myonecrosis Virus (IMNV)
   a. DNA target region: ORF 1

| SEQ ID NO: 7 | |
|---|---|
| GATTTTGTAA ATGGGACCCA ACCTACATTG AAATTTCTGA TTGTATGCTG CCACCTCCAG | 60 |
| ACCTTACATC GTGCGGGGAC GTTGAGAGTA ATCCTGGACC TATCATACAT AGCGTTGCAT | 120 |
| TTGCAAGAAC TGGTTCAGTA TGGACACCTG CCACCTTTAC TTTCAATACT ACATCATCCC | 180 |
| CGGGTAGACT GCAAGTACAA ATGTCATCCA GCGACAATAG ATATGGGTTC AATTCTGTTT | 240 |

b. Primer regions

TABLE 10

| | | | | | SEQ | | | | | |
|---|---|---|---|---|---|---|---|---|---|---|
| Primer | | | | | ID | Length | | Hairpin | Self Dimer | |
| name | start | end | Direction | Sequence (with extension) | NO: | (bp) | % GC | Tm | Tm | Tm |
| B3 | 200 | 217 | reverse | TTGTCGCTGGATGACATT | 73 | 18 | 44.4 | 45.1 | None | 54.2 |
| BIP 1 | 116 | 138 | forward | TGCATTTGCAAGAACTGGTTCAG | 74 | 23 | 43.5 | 49.7 | 14.7 | 60.5 |
| BIP 2 | 181 | 198 | reverse | GTACTTGCAGTCTACCCG | 75 | 18 | 55.6 | None | None | 54.5 |
| F3 | 28 | 50 | forward | TTGAAATTTCTGATTGTATGCTG | 76 | 23 | 30.4 | None | 18.6 | 53.9 |
| FIP 1 | 93 | 115 | reverse | ACGCTATGTATGATAGGTCCAGG | 77 | 23 | 47.8 | None | None | 59.2 |
| FIP 2 | 53 | 71 | forward | ACCTCCAGACCTTACATCG | 78 | 19 | 52.6 | None | None | 55.8 |
| LB | 148 | 172 | forward | CTGCCACCTTTACTTTCAATACTAC | 79 | 25 | 40 | None | None | 57.9 |
| LF | 74 | 92 | reverse | ATTACTCTCAACGTCCCCG | 80 | 19 | 52.6 | 40.8 | None | 56.6 |

c. Mapping of primers within the gene
See e.g., FIG. 17B.
6. Vibrio spp.-causing Acute Hepatopancreatic Necrosis Disease/Early Mortality Syndrome
   a. DNA target region: Pir A & Pir B

| Pir A | |
|---|---|
| SEQ ID NO: 190 | |
| ATGAGTAACA ATATAAAACA TGAAACTGAC TATTCTCACG ATTGGACTGT CGAACCAAAC | 60 |
| GGAGGCGTCA CAGAAGTAGA CAGCAAACAT ACACCTATCA TCCCGGAAGT CGGTCGTAGT | 120 |
| GTAGACATTG AGAATACGGG ACGTGGGGAG CTTACCATTC AATACCAATG GGGTGCGCCA | 180 |
| TTTATGGCTG GCGGCTGGAA AGTGGCTAAA TCACATGTGG TACAACGTGA TGAAACTTAC | 240 |
| CATTTACAAC GCCCTGATAA TGCATTCTAT CATCAGCGTA TTGTTGTAAT TAACAATGGC | 300 |
| GCTAGTCGTG GTTTCTGTAC AATCTATTAC CACTAAGAAG GTGCTCACAT GACTAACGAA | 360 |
| Pir B | |
| SEQ ID NO: 191 | |
| ATGAGTAACA ATATAAAACA TGAAACTGAC TATTCTCACG ATTGGACTGT CGAACCAAAC | 60 |
| GGAGGCGTCA CAGAAGTAGA CAGCAAACAT ACACCTATCA TCCCGGAAGT CGGTCGTAGT | 120 |
| GTAGACATTG AGAATACGGG ACGTGGGGAG CTTACCATTC AATACCAATG GGGTGCGCCA | 180 |
| TTTATGGCTG GCGGCTGGAA AGTGGCTAAA TCACATGTGG TACAACGTGA TGAAACTTAC | 240 |
| CATTTACAAC GCCCTGATAA TGCATTCTAT CATCAGCGTA TTGTTGTAAT TAACAATGGC | 300 |
| GCTAGTCGTG GTTTCTGTAC AATCTATTAC CACTAAGAAG GTGCTCACAT GACTAACGAA | 360 |
| TACGTTGTAA CAATGTCATC TTTGACGGAA TTTAACCCTA ACAATGCTCG TAAAAGTTAT | 420 |
| TTATTTGATA ACTATGAAGT TGATCCTAAC TATGCTTTCA AAGCAATGGT TTCATTTGGT | 480 |
| CTTTCAAATA TTCCTTACGC GGGTGGTTTT TTATCAACGT TATGGAATAT CTTTTGGCCA | 540 |
| AATACGCCAA ATGAGCCAGA TATTGAAAAC ATTTGGGAAC AATTACGTGA CAGAATCCAA | 600 |
| GATTTAGTAG ATGAATCGAT TATAGATGCC ATCAATGGAA TATTGGATAG CAAAATCAAA | 660 |
| GAGACACGCG ATAAAATTCA AGACATTAAT GAGACTATCG AAAACTTCGG TTATGCTGCG | 720 |
| GCAAAAGATG ATTACATTGG TTTAGTTACT CATTACTTGA TTGGACTTGA AGAGAACTTT | 780 |
| AAGCGCGAGC TAGACGGTGA TGAATGGCTT GGTTATGCGA TATTGCCTCT ATTAGCAACA | 840 |
| ACTGTAAGTC TTCAAATTAC TTACATGGCT TGTGGTCTGG ATTATAAGGA TGAATTCGGT | 900 |
| TTCACCGATT CTGATGTGCA TAAGCTAACA CGTAATATTG ATAAGCTTTA TGATGATGTA | 960 |
| TCGTCTTACA TTACAGAACT CGCTGCGTGG GCTGATAACG ACTCTTACAA TAATGCAAAC | 1020 |
| CAAGATAACG TGTATGATGA AGTGATGGGT GCTCGTAGTT GGTGTACGGT TCACGGCTTT | 1080 |
| GAACATATGC TTATTTGGCA AAAAATCAAA GAGTTGAAAA AAGTTGATGT GTTTGTTCAC | 1140 |
| AGTAATTTAA TTTCATATTC ACCTGCTGTT GGTTTTCCTA GTGGTAATTT CAACTATATT | 1200 |
| GCTACAGGTA CGGAAGATGA AATACCTCAA CCATTAAAAC CAAATATGTT TGGGGAACGT | 1260 |
| CGAAATCGTA TTGTAAAAAT TGAATCATGG AACAGTATTG AAATACATTA TTACAATCGC | 1320 |
| GTAGGTCGAC TTAAACTAAC TTATGAAAAT GGGGAAGTGG TAGAACTAGG CAAGGCTCAT | 1380 |
| AAATATGACG AGCATTACCA ATCTATTGAG TTAAACGGCG CTTACATTAA ATATGTTGAT | 1440 |
| GTTATTGCCA ATGGACCTGA AGCAATTGAT CGAATCGTAT TTCATTTTTC AGATGATCGA | 1500 |
| ACATTTGTTG TTGGTGAAAA CTCAGGCAAG CCAAGTGTGC GTTTGCAACT GGAAGGTCAT | 1560 |
| TTTATTTGTG GCATGCTTGC GGATCAAGAA GGTTCTGACA AAGTTGCCGC GTTTAGCGTG | 1620 |
| GCTTATGAAT TGTTTCATCC CGATGAATTT GGTACAGAAA AGTAGTAA | 1668 |

b. Primer regions Pir A (Table 11A) and Pir B (Table 11B)

TABLE 11A
Pir A

| | | | | | SEQ | | | | Self | |
|---|---|---|---|---|---|---|---|---|---|---|
| Primer | | | | Sequence | ID | Length | | Hairpin | Dimer | |
| name | start | end | Direction | (with extension) | NO: | (bp) | %GC | Tm | Tm | Tm |
| B3 | 301 | 318 | reverse | ACAGAAACCACGACTAGC | 81 | 18 | 50 | None | None | 54.3 |
| BIP 1 | 215 | 237 | forward | ATGTGGTACAACGTGATGAAACT | 82 | 23 | 39.1 | 44.2 | None | 58.6 |
| BIP 2 | 271 | 294 | reverse | GTTAATTACAACAATACGCTGATG | 83 | 24 | 33.3 | None | None | 55 |
| F3 | 123 | 142 | forward | AGACATTGAGAATACGGGAC | 84 | 20 | 45 | None | None | 54.6 |
| FIP 1 | 192 | 213 | reverse | TGATTTAGCCACTTTCCAGCCG | 85 | 22 | 50 | None | None | 61.2 |
| FIP 2 | 143 | 160 | forward | GTGGGGAGCTTACCATTC | 86 | 18 | 55.6 | 39.3 | 12.7 | 54.6 |
| LB | 238 | 258 | forward | TACCATTTACAACGCCCTGAT | 87 | 21 | 42.9 | None | None | 57.4 |
| LF | 168 | 183 | reverse | AAATGGCGCACCCCAT | 88 | 16 | 56.3 | 47.4 | 10.8 | 56.4 |

TABLE 11B
PirB

| | | | | | SEQ | | | | Self | |
|---|---|---|---|---|---|---|---|---|---|---|
| Primer | | | | Sequence | ID | Length | | Hairpin | Dimer | |
| name | start | end | Direction | (with extension) | NO: | (bp) | %GC | Tm | Tm | Tm |
| F3-PirB | 924 | 947 | forward | GCTAACACGTAATA | 125 | 24 | 33.3 | None | None | 55.2 |
| | | | | TTGATAAGCT | | | | | | |
| F2-PirB | 958 | 981 | forward | GTATCGTCTTACATT | 131 | 24 | 37.5 | None | None | 54.9 |
| | | | | ACAGAACTC | | | | | | |
| LF-PirB | 989 | 1009 | reverse | TGTAAGAGTCGTTA | 129 | 21 | 47.6 | None | None | 57.2 |
| | | | | TCAGCCC | | | | | | |
| F1c-PirB | 1013 | 1037 | reverse | TCATACACGTTATC | 132 | 25 | 36 | None | None | 59.4 |
| | | | | TTGGTTTGCAT | | | | | | |
| B1c-PirB | 1040 | 1059 | forward | AAGTGATGGGTGCT | 134 | 20 | 50 | None | None | 58.7 |
| | | | | CGTAGT | | | | | | |
| LB-PirB | 1060 | 1078 | forward | TGGTGTACGGTTCA | 130 | 19 | 57.9 | 49.8 | 11.1 | 61.5 |
| | | | | CGGCT | | | | | | |
| B2-PirB | 1079 | 1100 | reverse | TGCCAAATAAGCAT | 133 | 22 | 31.8 | 34.7 | 4.3 | 54.7 |
| | | | | ATGTTCAA | | | | | | |
| B3-PirB | 1107 | 1131 | reverse | CACATCAACTTTTTT | 126 | 25 | 32 | None | None | 56.3 |
| | | | | CAACTCTTTG | | | | | | |

c. Mapping of primers within the gene
See e.g., FIG. 12B (PirA) and FIG. 18B (PirB).
7. Internal control Penaeus spp.
   a. DNA target region: 18SrRNA

| SEQ ID NO: 8 | |
|---|---|
| TGCCTTATCA GCTCTCGATT GTAGGTTAAA CGCCTACAAT GGCTATCACG GGTAACGGGG | 60 |
| AATCAGGGTT CGATTCCGGA GAGGGAGCCT GAGAAACGGC TACCACATCT AAGGAAGGCA | 120 |
| GCAGGCGCGC AAATTACCCA CTCCCGGCAC GGGGAGGTAG TGACGAAAAA TACTGTTGCG | 180 |
| AGCCCCGAAC GGGGCCTCGC AATTGGAATG AGTACACTTT AAATCCTTGT ACGAGGATCG | 240 |
| AGTGGAGGGC AAGTCTGGTG CCAGCCGCCG CGGTAATTCC AGCTCCACTA GCGTATATTA | 300 |
| AAGTTGTTGC GGTTGAAACG CTCGTAGTTT GACTTCTGCT CCGGACCGGC GGTCCGCCTT | 360 |
| AGCGGCGGCT ACTGCCGGGT TCCGAGCTGT GTCCCCGCCG GCGCACATGG GGTTTTTATG | 420 |
| CCCTTAACCG GGTGTCCCCT TGTGGCCGGC ACGTTTACTT TGAAAAAATT AGAGTGCTCA | 480 |
| GAGCAGGCTG GTTTTTGCTT ACAGCCCGAA TGGTCGTGCA TGGAATGATG GAACAGGACC | 540 |
| TCGGTTCTAT TTTGTCGGTT TTTCGGAACC CGAGGTAATG ATTAATAGAA GCAGACGGGG | 600 |
| GCATTCGTAC TGCGACGCTA GAGGTGAAAT TCTTAGACCG TCGCATGACG ACCTACTGCG | 660 |
| AAAGCATCTG AGGATGTTTT CATTGATCAA GAACGAAAGT TAGAGGTTCG AAGGCGATCA | 720 |
| GATACCGCCC TAGTTCTAAC CTTAAACGAT GCTGACTAGC GATCCGCCGC AGTTATTCCC | 780 |
| ATGACCCGGC GGGTAGCTTC CGGGAAACCA AAGTCTTTGA GTTCCGGGGG AAGTATGGTT | 840 |
| GCAAAGCTGA A | 851 |

b. Primer regions

TABLE 12

| | | | | | SEQ | | | | Self | |
|---|---|---|---|---|---|---|---|---|---|---|
| Primer | | | | Sequence | ID | Length | | Hairpin | Dimer | |
| name | start | end | Direction | (with extension) | NO: | (bp) | %GC | Tm | Tm | Tm |
| B3 | 270 | 288 | reverse | GTGGAGCTGGAATTACCGC | 89 | 19 | 57.9 | 42.6 | None | 58.6 |
| BIP 1 | 187 | 206 | forward | GAACGGGGCCTCGCAATTGG | 90 | 20 | 65 | None | 28.8 | 64.4 |
| BIP 2 | 238 | 255 | reverse | GACTTGCCCTCCACTCGA | 91 | 18 | 61.1 | None | None | 58.6 |
| F3 | 56 | 73 | forward | CGGGGAATCAGGGTTCGA | 92 | 18 | 61.1 | None | None | 58.7 |
| FIP 1 | 147 | 167 | reverse | TTCGTCACTACCTCCCCGTGC | 93 | 21 | 61.9 | None | None | 64.1 |
| FIP 2 | 89 | 108 | forward | CTGAGAAACGGCTACCACAT | 94 | 20 | 50 | None | None | 57.6 |
| LB | 212 | 236 | forward | GTACACTTTAAATCCTTGTACGAGG | 95 | 25 | 40 | 34.8 | 21.8 | 57.7 |
| LF | 109 | 126 | reverse | GCCTGCTGCCTTCCTTAG | 96 | 18 | 61.1 | None | None | 57.8 |

c. Mapping of primers within the gene
See e.g., FIG. 14B.
8. Enterocytozoon hepatopenaei (EHP)
   a. DNA target region: Beta tubulin

| (Beta tubulin) | |
|---|---|
| SEQ ID NO: 192 | |
| CACTACACAG AGGGTGCGGA ACTATGCGAA CAGGTGTTGG AATGCATAAG AAAGGAAGCA | 60 |
| GAAAAAACAG ACTGTCTTCA GGGATTCCAA CTTACACACT CCTTGGGTGG TGGAACAGGT | 120 |
| TCGGGAATGG GAACATTGCT GGTATCAAAA ATAAAGGAGG AATTTCCAGA TAGGATGCTT | 180 |
| GCAACATTTT CTGTAGTTCC AAGTCCAAAA GTTTCTGACA CTGTGGTTGA ACCATACAAT | 240 |
| GCAACACTCT CTTTTCACCA GCTGGTTGAA AATGCAAATC AAACATTTTG CATCGATAAT | 300 |
| GACGCGCTTT ATGAAATATG CACAAAAACA CTCAAATTAA AAAATCCATC TTATGACAAT | 360 |
| TTAAACAGTC TTGTGTCCAA AGTTATGAGT GGAATTACCA CTTGTTTTAG ATTTCCAGGT | 420 |
| CAACTTAACT CAGATCTGAG AAAATTGGCG GTTAACATGA TTCCATTTCC ACGACTGCAC | 480 |
| TTTTTCTGTG TAGGATATGC GCCTCTGTGT TCAGAAGCGT CTTCAAGCTA CAGAAATCTT | 540 |
| ACGGTTGCAG ATTTGACAGC ACAGCTTTTT GACAGCAAAA ACATGATGAC TGCGTGTGAC | 600 |
| CCAAGAAAAG GAAGGTATTT GACAGCAGCT GTTTATTTTA GAGGGAAAAT GTCAATGAAA | 660 |
| GAAGTAGACG AGCAGATGAA TTTGGCACAA ACAAGAACTA GTGATTCTTT TGTCGAGTGG | 720 |
| ATTCCAAACA ACGTGCAAAC GGCTGTTTGT AACGTAGCAC CAAATGACGT AGAAATGAGC | 780 |
| GCTACATTCA TAGGCAACAC AACTTCAATT CAAGAGATTT TCATGCGTCT TGGTGAGCAG | 840 |
| TTTTCAAGCA TGTTTAAACG AAAGGCATTT | |

a. Primer regions

TABLE 13

| | | | | | SEQ | | | | Self | |
|---|---|---|---|---|---|---|---|---|---|---|
| | | | | Sequence | ID | | | Hairpin | Dimer | |
| Name | start | end | Direction | (with extension) | NO: | Length | %GC | Tm | Tm | Tm |
| F3 | 503 | 520 | forward | CTCTGTGTTCAG | 135 | 18 | 50 | 48 | None | 54.6 |
| | | | | AAGCGT | | | | | | |
| F2 | 521 | 543 | forward | CTTCAAGCTACA | 141 | 23 | 39.1 | None | None | 55.5 |
| | | | | GAAATCTTACG | | | | | | |
| LF | 544 | 563 | reverse | TGTGCTGTCAAA | 139 | 20 | 45 | 42.1 | None | 57.2 |
| | | | | TCTGCAAC | | | | | | |
| F1c | 564 | 588 | reverse | CATCATGTTTTT | 142 | 25 | 36 | 52.5 | 17.1 | 59.1 |
| | | | | GCTGTCAAAAA | | | | | | |
| | | | | GC | | | | | | |
| B1c | 589 | 608 | forward | ACTGCGTGTGAC | 144 | 20 | 50 | None | None | 60.1 |
| | | | | CCAAGAAA | | | | | | |
| LB | 610 | 634 | forward | GGAAGGTATTT | 140 | 25 | 44 | 45.4 | 28.8 | 62 |
| | | | | GACAGCAGCTG | | | | | | |
| | | | | TTT | | | | | | |
| B2 | 641 | 663 | reverse | TTCTTTCATTGA | 143 | 23 | 34.8 | None | None | 55.4 |
| | | | | CATTTTCCCTC | | | | | | |
| B3 | 664 | 684 | reverse | CAAATTCATCTG | 136 | 21 | 42.9 | None | None | 54.8 |
| | | | | CTCGTCTAC | | | | | | |

b. Mapping of primers within the gene
See e.g., FIG. 19.
9. Infectious Myonecrosis virus (IMNV)
   a. DNA target region: RdRp
   SEQ ID NO: 193

| | |
|---|---|
| GACGTATTTC AACAAAGGCG TGGGACACAG CATATGATAG TAACATCCAA GCATATCAGG | 60 |
| ACATGCATTA TCAAATGTTT TCGTGGAGTT CAATGCAAGG TAGTATTGCG CCAGCAATGG | 120 |
| TGGACGAAAT TCTTCATAAC CTTTGTGGCC AAATGTTTGG CTTCAGCTTA CCATTGAGAC | 180 |
| AAGTCTTATT TAACGCATTG CCAATCACTT TTTCATCGTT TGGAAGTTGG ATGTTGCCTA | 240 |
| GAGTTTCTGA TGGTTTCCAA ACTGTAAGGT ATTATGATGC AGGTCCACCA GCCATTAATG | 300 |
| CAAAACGTGA TGGGGAAGTA CCAGTAAGTA TGATTGACGC ATGGACCTAT AAATTTACAG | 360 |
| AAAAATTGCC AAAAAGTTTT TTGCCATGGC CAATGCCAGA AGGAAAGGAC AGTACAATGG | 420 |
| GATATGATCC GGAAAAAGAA CCAGCACTAA TTGATAATTC AAATGAGACA GGCAATGTAT | 480 |
| TCAGACCATT CATGGCAAGA AATGGCAACA ATTCCAATTA TTTACCAACC AACTACACAA | 540 |
| TTGACGTATC ACAGAATGGT CATGATGAGA GTTGTATTAA TGTTGACCTT TTTAACAATG | 600 |
| TTGCAGGAGT AACACTAACA AATTATGATG GAACCGCAAC AAACGCAAAC GTCGTACCAA | 660 |
| CAGGATCATA CATTAAGCAG AGAGCAATGC CTATTAATGC AAATGCGGTA CGACCAACTG | 720 |
| AAACACTCGA CGCTGCTAAC CATACAAAAC CTTTTGCTAT TGAAGGAGGA AGACTCGTAT | 780 |
| ATTTGGGTGG AACAATTGCA AATACAACCA ATGTGGTAAA CGCGATGCAG AGGAAACAAA | 840 |
| GGCTTTCAAA ACCGGCATTC AAGTGGGCAC ATGCTCAGAG ACAACGTGTA TATGACAGCA | 900 |
| GTCGTCCAGG GATGGACGCA ATCACAAAGT TGTGTGCACG AAAGTCGGGT TTTATGAATG | 960 |
| CCCGTTCCAC AGCAATGATG GCACCCAAGA CTGGACTCAG CGCTGTTATA GATCAAGCAC | 1020 |
| CAAATACATC TCAAGACTTG ATCGAACAGC CGAGTCAGCA AGAGGTTATG GATATGCAAG | 1080 |
| CGACAGCAAC AGTATAAATC AGATATATCA AATTGCATTG CATAGAAAGG CAAAACTTAT | 1140 |
| AACAGCAAAG AAATGGCAAG AATTAACAAA AGGTATTTAT AATGCATCTA CCCTGACACC | 1200 |
| GAAGATAGTT GACCAAATTA TAAAGGATGA AGGAAGTGGG ACCGATAAGA CAAAATATGT | 1260 |
| AAATGTTCCT AAAATAATTA CTGACAAAGA ATTACAAACA TTCTATGTAC CAAGAAGCAA | 1320 |
| CGCAGACCTA GTTATAAGAA GAATACGTTT AATCGACCTT TGGCGTAACC TAAAACCAGA | 1380 |
| TCAAATGGAC GAGATTCGTA ATTACACTCA TCTAGATTAT ATCTTTGTAC AAAACATTTG | 1440 |
| TATCTATATG TTAGTATTTG GAATAGACAC AGTTAAACAC TTTAGACAAA TAGGTCTATT | 1500 |
| C | 1501 |

b. Primer regions

TABLE 14

| | | | | | | SEQ | | | Self | |
|---|---|---|---|---|---|---|---|---|---|---|
| | | | | | Sequence | ID | | Hairpin | Dimer | |
| Name | start | end | Length | Direction | (with extension) | NO: | %GC | Tm | Tm | Tm |
| F3 | 857 | 874 | 18 | forward | ATTCAAGTGGGC | 146 | 50 | 41.1 | None | 55.9 |
| | | | | | ACATGC | | | | | |
| F2 | 877 | 897 | 21 | forward | AGAGACAACGTG | 152 | 38.1 | 34.9 | None | 54.2 |
| | | | | | TATATGACA | | | | | |
| LF | 898 | 914 | 17 | reverse | CATCCCTGGACG | 150 | 64.7 | None | None | 57 |
| | | | | | ACTGC | | | | | |
| F1C | 917 | 937 | 21 | reverse | GCACACAACTTT | 153 | 47.6 | 56.9 | 25.3 | 59.5 |
| | | | | | GTGATTGCG | | | | | |
| B1C | 939 | 961 | 23 | forward | CGAAAGTCGGGT | 155 | 43.5 | 36 | None | 58.9 |
| | | | | | TTTATGAATGC | | | | | |
| LB | 982 | 1000 | 19 | forward | CACCCAAGACTG | 151 | 57.9 | 46.9 | None | 57.1 |
| | | | | | GACTCAG | | | | | |
| B2 | 1001 | 1020 | 20 | reverse | GTGCTTGATCTAT | 154 | 45 | None | None | 54.3 |
| | | | | | AACAGCG | | | | | |
| B3 | 1026 | 1045 | 20 | reverse | TCGATCAAGTCTT | 147 | 40 | 44 | 19.6 | 53.8 |
| | | | | | GAGATGT | | | | | |

c. Mapping of primers within the gene
See e.g., FIG. 20.
10. Vibrio parahaemolyticus.
   a. DNA target region: ToxR

| SEQ ID NO: 194 | |
|---|---|
| TCATCCATCT AAGAAGAACT AAATGACTAA CATCGGCACC AAATTTCTAC TTGCTCAAAG | 60 |
| GTTTACCTTT GATCCAAATA GTAATTCGCT CGCTGACCAA CAAAGCGGCA ACGAAGTTGT | 120 |
| ACGATTAGGA AGCAACGAAA GCCGTATACT CCTGATGTTG GCGGAGAGAC CAAACGAAGT | 180 |
| TTTAACCCGT AACGAGCTTC ACGAGTTTGT TTGGCGTGAG CAAGGTTTTG AGGTGGATGA | 240 |
| CTCAAGCCTG ACTCAAGCGA TTTCTACTCT GCGTAAGATG TTGAAGGATT CAACCAAATC | 300 |
| TCCAGAGTTT GTTAAAACCG TTCCAAAACG AGGCTATCAA CTCATTTGTA CTGTTGAACG | 360 |
| CCTAAGCCCG CTTTCTTCAG ACTCAAGCTC AATTGAAGTT GAAGAGCCAG CTTCTGATAA | 420 |
| CAATGACGCC TCTACTAATG AGGTAGAAAC GATCGTAGAG CCGTCTTTAG CGACGACTTC | 480 |
| TGACGCAATC GTTGAACCAG AAGCGCCAGT AGTACCTGAA AAAGCACCTG TGGCTTCTGC | 540 |
| TGTGAATCCT TGGATTCCAC GCGTTATTTT ATTTTTGGCA CTATTACTAC CGATTTGCGT | 600 |
| ACTGCTGTTT ACAAACCCAG CGGAATCTCA GTTCCGTCAG ATTGGTGAGT ATCAGAACGT | 660 |
| ACCAGTGATG ACACCTGTAA ATCACCCGCA AATCAACAAC TGGTTGCCTT CTATTGAGCA | 720 |
| GTGCATTGAA CGCTACGTTA AGCACCATGC AGAAGACTCG TTACCAGTGG AAGTGATTGC | 780 |
| CACTGGCGGA CAAAATAACC AGCTGATTTT GAACTACATT CATGACAGCA ACCACTCGTA | 840 |
| TGAGAACGTG ACATTGCGTA TTTTCGCAGG TCAAAACGAT CCAACAGACA TCTGCAAATA | 900 |
| AAGGAGGCCA GCATGAAGAT TAAAGTAGCA TCTGCGGTTT TGGCCGTATC TATCCTTTTC | 960 |
| AGTGGTTGGT TGTACTGGGG CAGTGACCTT AAAGTTGAGC AAGTGCTTAC ATCAAATGAA | 1020 |
| TGGCAGTCAA CCATGGTGAC TGTAATTACT GATAACTTGC CAGACGATAC CGTAGGCCCG | 1080 |
| TTACGTCGAG TGAATGTGGA GTCGAACGTT AAATACCTGC CGAATGGCGA TTACATTCGC | 1140 |
| GTGGCAAACA TCAAACTGTT CGCACAAGGC TCGACGGCTG AATCGACAAT TAATATTTCA | 1200 |
| GAGAAAGGTC GCTGGGAAGT GAGTGATAAC TATCTGCTTG TTTCTCCTTC TGAGTTCAAA | 1260 |
| GATATTTCTT CTTCTCAATC CAAGGATTTT TCTGAAGCGC AACTACGTTT AATTACTCAA | 1320 |
| ATCTTTAAGC TAGATGCAGA ACAAAGCCGC CGAATTGACG TGGT | 1364 |

b. Primer regions

TABLE 15

| | | | | | SEQ | | | | Self | |
|---|---|---|---|---|---|---|---|---|---|---|
| | | | | Sequence | ID | | | Hairpin | Dimer | |
| Name | start | End | Direction | (with extension) | NO: | Length | %GC | Tm | Tm | Tm |
| toxR | 23 | 901 | forward | | | | | | | |
| CDS | | | | | | | | | | |
| toxR | 23 | 901 | forward | | | | | | | |
| gene | | | | | | | | | | |
| F3 | 320 | 338 | forward | GTTCCAAAACG | 157 | 19 | 47.4 | None | None | 53.8 |
| | | | | AGGCTATC | | | | | | |
| F2 | 343 | 362 | forward | CATTTGTACTG | 163 | 20 | 45 | None | None | 55.6 |
| | | | | TTGAACGCC | | | | | | |
| LF | 363 | 381 | reverse | TCTGAAGAAA | 161 | 19 | 47.4 | 34.8 | 6.3 | 56.4 |
| | | | | GCGGGCTTA | | | | | | |
| F1c | 383 | 407 | reverse | GCTCTTCAACT | 164 | 25 | 40 | 39.7 | 18.9 | 60.3 |
| | | | | TCAATTGAGCT | | | | | | |
| | | | | TGA | | | | | | |
| B1c | 421 | 444 | forward | CAATGACGCCT | 166 | 24 | 45.8 | 49.3 | 10.9 | 60.2 |
| | | | | CTACTAATGAG | | | | | | |
| | | | | GT | | | | | | |
| LB | 458 | 475 | forward | GAGCCGTCTTT | 162 | 18 | 61.1 | 51.9 | 6.4 | 58.3 |
| | | | | AGCGACG | | | | | | |
| B2 | 486 | 504 | reverse | GCTTCTGGTTC | 165 | 19 | 47.4 | None | None | 54.7 |
| | | | | AACGATTG | | | | | | |
| B3 | 505 | 524 | reverse | CTTTTTCAGGT | 158 | 20 | 45 | 39 | None | 53.6 |
| | | | | ACTACTGGC | | | | | | |

c. Mapping of primers within the gene
See e.g., FIG. 21B.
11. Tilapia lake virus (TiLV)
   a. DNA target region: Segment 3

| SEQ ID NO: 195 | |
|---|---|
| GCAAATTTTT CCCATAATCC TCTATTAGAA CGTCGTAACC TTTAGCGAAG GCGTCGAAAG | 60 |
| CGATCATCTC GCAAATGGGT GTACTGTCAT CCGCAATCTT ACTGCACAAA GTGAATAATA | 120 |
| AAGTGAGCTT AAGGGTATTG TACCCCTTAT CTCAGAAGCC AGCTGGTAGC CTTTCCTGAA | 180 |
| CTCGTCCTTA CAGACGCTAA GTGCTAGCCG GTGCCTATTA AAATGTTGAG CCACTGCTTG | 240 |
| GCTAACCCTT GTAGAGTCGA GGCATTCCAG AAGTAAGATG ACGTCCCATC TTGTCTCAAG | 300 |
| ACCACTAGCT CTGTCCAGAT CACCCTTCCT ACTTATGGGA GGTAGTTCCA ACATATCCAG | 360 |
| CTTGTAAATT TCTCGGGTAC TCACAAAGTC TTGCTCCCCT ATATTAGCCG ATTGCTTGGG | 420 |
| ATCTAGGTGC ATCACATGCA CAGCTGCCCT GTACCCGTCA AACTCAAAAT CGTGTTCACA | 480 |
| RCCAGGTTTA CTTAGACCTA CAACTAAGTG GTGAGTGGAG GCGGTTGGTC TCCTTTTACT | 540 |
| GTGCTTTCCA GAGTCGCGCA TGACCGGTAC AGCTAGTATG CTGGTATTAT CGCTATGCAG | 600 |
| TACTTTCCCT GCCTGAGTTG TGCTTCTAGC AATCAACATC AAAAGCTCAC GAGCAAGTGG | 660 |
| RGCACTAGCC GGTAGAGGTA ATATCTTCTG TGTAGCAGGC TTRTGAGAAG CAACTGTATA | 720 |
| CCTTTGTATC CACCCTCCAT TGCGGAACTC AAATTCTCTA TCACGTGCGT ACTCGTTCAG | 780 |
| TATAAGTTCT CTTGCCTCTT GGTCAAGACC ACACTCCTCA CCGCAGGCGA GGAACTTTGA | 840 |
| GCACTCGAAG AATCCATATT GCCTCTTTAG CTCAGCTGTT TCCTTGGATA TGTCCGCAAG | 900 |
| TCTGGGTGGT GCCACCCACT CAATACGAGG CTTCGGGCCA CTCTTTGGAT GTGGTAGTTC | 960 |
| CAATAGCCGT TCCCTTAGCT CAGCATCGTA GGATGCCTTG TGCCCAACTT TAACAACCCC | 1020 |
| GTCGATCTCC AAATAATCCT TGAATTGTAC CTTACAAATC TGCTGGTACT CTTGCTCAGA | 1080 |
| GCAAACGTAT GTAGCATCAT TGTTCAAAAT GCAACGAACT GTTGCCTTTG GAAATTTCCG | 1140 |
| CGGCTGGCCT TCCAGCTCAA AGGCAATCCA CTTATTCTTC AAACAGTCAT AACAGTCACC | 1200 |
| CTCTACGGGG ACTCCTGGAC GTCTCTCTAC TGTACTGTAA GAACTTAGGA ACCTTCGGCT | 1260 |
| CCCTTCGCTA TAAGTGAAAT CGTCACAGAA AACCCCAGTT AGCTGTGCAA ACCGCGAGTC | 1320 |
| CATTTCAAAA GAAAGTTAAC GGTCTATTAA GGATTAAGGG GTAATATTTG G | 1371 |

b. Primer regions

TABLE 16

| | | | | | SEQ | | | | Self | |
|---|---|---|---|---|---|---|---|---|---|---|
| | | | | Sequence | ID | | | Hairpin | Dimer | |
| Name | start | end | Direction | (with extension) | NO: | Length | %GC | Tm | Tm | Tm |
| B3 | 733 | 750 | forward | CCCTCCATTGCGGAACTC | 169 | 18 | 61.1 | 45.8 | None | 57.8 |
| B2 | 763 | 781 | forward | ACGTGCGTACTCGTTCAGT | 176 | 19 | 52.6 | 42.9 | 1.2 | 59.3 |
| LB | 787 | 806 | reverse | TTGACCAAGAGGCAAGAGAA | 173 | 20 | 45 | 39.1 | None | 56.7 |
| B1c | 807 | 826 | reverse | CTGCGGTGAGGAGTGTGGTC | 177 | 20 | 65 | 33.2 | None | 62.8 |
| F1c | 827 | 848 | forward | GCGAGGAACTTTGAGCACTCGA | 175 | 22 | 54.5 | 47.6 | 15 | 63.1 |
| LF | 866 | 886 | forward | TTTAGCTCAGCTGTTTCCTTG | 172 | 21 | 42.9 | 40.1 | 10.3 | 56.5 |
| F2 | 890 | 907 | reverse | ACCCAGACTTGCGGACAT | 174 | 18 | 55.6 | None | None | 58.5 |
| F3 | 910 | 928 | reverse | TCGTATTGAGTGGGTGGCA | 168 | 19 | 52.6 | None | None | 58.6 |

c. Mapping of primers within the gene
See e.g., FIG. 22B.
12. Tilapia lake virus (TiLV)
   a. DNA target region: Segment 8

| SEQ ID NO: 189 | |
|---|---|
| ATGGCTCAAA TCCCAACACT AAGAGAGGGC CAAGGGAAGC TCTACGATTT CACGCTCAAC | 60 |
| GGCATGACAG TGACTAGAGA CACAGTCAAC ACTGTAGTTG CTCTGGAGTT TCTTGTCAAT | 120 |
| GCAGGTCCGG ATTTGCTTTC CCTAACAATT GGCGAAGGCC TCTCAGAAGA AACAAAGTTT | 180 |
| AAACACCTGC TTGTTAAGCA CGCCGGCATG ACCCGAAAGC GGATAGAGGA AAGGCTGGGA | 240 |
| CGAATCTCGA GGCGAGTCAG TGTGACAGTC GACGCAATTA TAATAACAAA CCGCAAGGGT | 300 |
| CAAAGATTTG AATTCAATCG GAAGCAGTAC CTGGATATTG CCAAACAAGC TATGAAGCTT | 360 |
| AAGCTCCCTG GGATTAACTG TGTCGACATA CCCACTGCGC TCGCTTTTCT CGAGGAGGTC | 420 |
| CTGGCAACTG CTTTGAAGGA CACTGAAGGT TCACAAGATG ACAGGATGGC CCTTAAGGCA | 480 |
| GACACTTCTG CTGCTATCAA TCATTTCCGT GAAATGCTTA AATAA | |

b. Primer regions

TABLE 17

| | | | | | SEQ | | | | Self | |
|---|---|---|---|---|---|---|---|---|---|---|
| | | | | Sequence | ID | | | Hairpin | Dimer | |
| Name | start | end | Direction | (with extension) | NO: | Length | %GC | Tm | Tm | Tm |
| F3 | 29 | 47 | forward | GCCAAGGGAAGCTCTACGA | 179 | 19 | 57.9 | None | None | 59.1 |
| F2 | 55 | 73 | forward | CTCAACGGCATGACAGTGA | 185 | 19 | 52.6 | None | None | 57.8 |
| LF | 78 | 98 | reverse | ACTACAGTGTTGACTGTGTC | 183 | 21 | 42.9 | 56.8 | 16.3 | 56.5 |
| | | | | T | | | | | | |
| F1c | 115 | 136 | reverse | GCAAATCCGGACCTGCATTG | 186 | 22 | 54.5 | 34.9 | 26.9 | 62.7 |
| | | | | AC | | | | | | |
| B1c | 148 | 169 | forward | ATTGGCGAAGGCCTCTCAGA | 188 | 22 | 54.5 | 60.5 | 5.2 | 62.6 |
| | | | | AG | | | | | | |
| LB | 186 | 204 | forward | CCTGCTTGTTAAGCACGCC | 184 | 19 | 57.9 | 57.7 | 11.8 | 59.8 |
| B2 | 211 | 229 | reverse | CCTCTATCCGCTTTCGGGT | 187 | 19 | 57.9 | 54.5 | 3.8 | 58.9 |
| B3 | 239 | 257 | reverse | ACTCGCCTCGAGATTCGTC | 180 | 19 | 57.9 | 54.6 | 21.1 | 59.3 |

c. Mapping of primers within the gene
See e.g., FIG. 23B.

### Example 4—Exemplary Primers for Detection of Shrimp/Fish Pathogens Using a DNA Extraction Method Based on Boiling

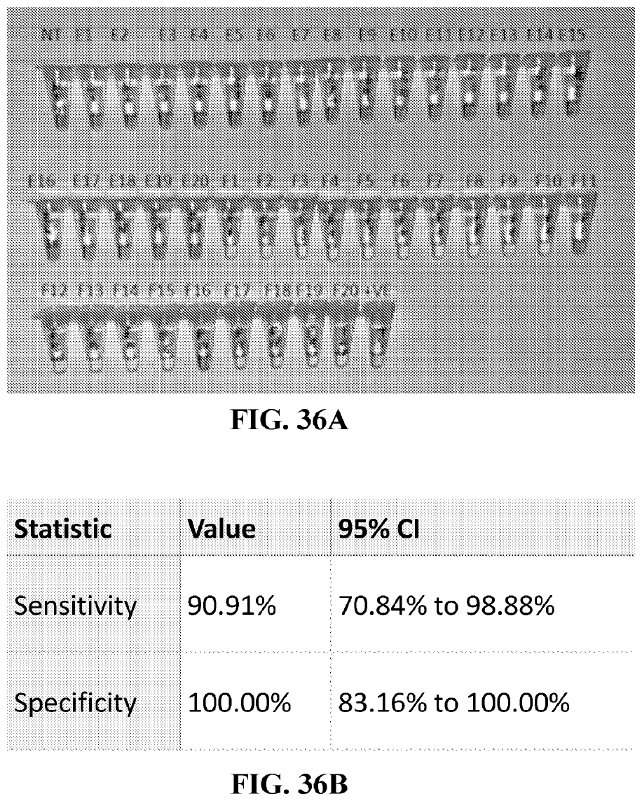

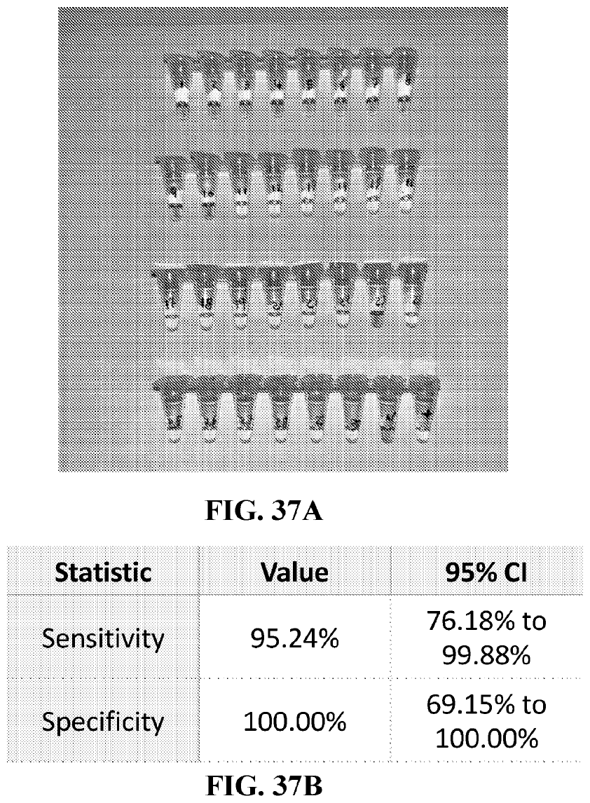

iii. Proof of Concept Demonstrated for:
1. White Spot Syndrome Virus (WSSV) (shrimp)
   d. Validation process
   See e.g., FIG. 36A-36B.
2. Infectious Myonecrosis Virus (IMNV) (Shrimp)
   a. Validation process
   See e.g., FIG. 37A-37B.

DNA extractions and detection of shrimp using the “boiling method”, which was as follows. 30 mg of P. vannamei muscle tissue infected with WSSV or IMNV were minced. 100 μL of TE buffer from Fisher BioReagents was added to the sample which was then macerated using the blunt end of a sterile wooden applicator. 20 μL of Proteinase K with a concentration of 600 mAU/mL from Qiagen was added, vortexed, and then centrifuged at 6,000 RPM at room temperature for 5 seconds using a USA Scientific mini-centrifuge to bring the contents of the tube down. One heat block was pre-heated to 65° C. and another was pre-heated to 95° C. The sample was incubated at 65° C. for 15 minutes and then placed in the heat block at 95° C. and incubated for 5 minutes. The sample was centrifuged at 10,500 rpm for 1 minute and 30 seconds at room temperature using a Corning LSE centrifuge. The supernatant was transferred to another tube from where an aliquot was taken to determine the optical density with a nanodrop and the pH of the extract was measured with a litmus paper.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

Further attributes, features, and embodiments of the present invention can be understood by reference to the following numbered aspects of the disclosed invention. Reference to disclosure in any of the preceding aspects is applicable to any preceding numbered aspect and to any combination of any number of preceding aspects, as recognized by appropriate antecedent disclosure in any combination of preceding aspects that can be made. The following numbered aspects are provided:

1. A kit configured to detect one or more shrimp pathogens using loop-mediate isothermal amplification (LAMP), the kit comprising:
one or more primer sets configured to specifically amplify one or more shrimp pathogen-specific target polynucleotides via LAMP; and one or more amplification reagents.
   2. The kit of aspect 1, further comprising one or more signal reagents.
   3. The kit of aspect 2, wherein at least one of the one or more signal reagents is a pH sensitive reagent that is capable of changing color from a first visible color to a second visible color in response to amplification of a target polynucleotide.
   4. The kit of aspect 3, wherein the first visible color and the second visible color are different colors.
   5. The kit of any one of aspects 3-4, wherein the pH sensitive indicator changes color when the LAMP amplification moves from a neutral or basic pH to an acid pH as the reaction proceeds.
   6. The kit of any one of aspects 3-5, wherein the pH sensitive indicator is phenol red, cresol red, phenolphthalein, methyl orange, thymol blue, bromothymol blue, or neutral red, or m-cresol purple.
   7. The kit of any one of aspects 3-6, wherein the signal reagent is capable of producing a signal detectable by the naked eye, an optical sensor, a camera, a smartphone, a tablet, an electronic portable device, or any combination thereof.
   8. The kit of any one of the aspects 1-7, wherein the one or more primer sets configured for LAMP amplification is/are specific for one or more the shrimp or fish pathogens selected from: White Spot Syndrome Virus (WSSV), Enterocytozoon hepatopenaei (EHP), Necrotising Hepatopancreatitis/Hepatobacter penaei (NHP-B), Infectious Hypodermal and Hematopoietic Necrosis Virus (IHHNV), Infectious Myonecrosis Virus (IMNV), Vibrio spp.-causing Acute hepatopancreatic necrosis disease (AHPND)/EMS), Yellow Head Virus (YHV), Taura Syndrome Virus (TSV), Vibrio parahaemolyticus, Tilapia Lake virus (TiLV), or any combination thereof.
   9. The kit of aspect 8, wherein one or more primers of the one or more primer sets is selected from one or more of those set forth or otherwise identified in any one or more of Tables 1 (SEQ ID NOs: 9-14), 3 (SEQ ID NOS: 17-24), 4 (SEQ ID NOS: 25-32), 5 (SEQ ID NOS: 33-40), 6 (SEQ ID NOS: 41-48), 7 (SEQ ID NOS: 49-56), 8 (SEQ ID NOS: 57-64), 9 (SEQ ID NOS: 65-72), 10 (SEQ ID NOS: 73-80), 11A (SEQ ID NOS: 81-88), 11B (SEQ ID NOS: 125-126, 129-134), 12 (SEQ ID NOS: 89-96), 13 (SEQ ID NOS: 135-136, 139-144), 14 (SEQ ID NOS: 146-147, 150-155), 15 (SEQ ID NOS: 157-158, 161-166), 16 (SEQ ID NOS: 168-169, 172-177), and/or 17 (SEQ ID NOS: 179-180, 183-188) and/or FIGS. 8A(SEQ ID NOS: 17-24)-8B(SEQ ID NO: 97), 9A(SEQ ID NOS: 65-72)-9B(SEQ ID NO: 98):, 10A(SEQ ID NOS: 33-40)-10B(SEQ ID NO: 99), 11A(SEQ ID NOS: 49-56)-11B(SEQ ID NO: 100), 12A(SEQ ID NOS: 81-88, 101-102)-12B(SEQ ID NO: 103), 13A(SEQ ID NOS: 25-27, 29-32, 38)-13B(SEQ ID NO: 104), 14A(SEQ ID NOS: 89-96)-14B(SEQ ID NO: 105), and 15A(SEQ ID NOS: 109-116)-15B(SEQ ID NO: 106), 16A(SEQ ID NOS: 117-124)-16B(SEQ ID NO: 107), 17A(SEQ ID NOS: 73-80)-17B(SEQ ID NO: 196), 18A(SEQ ID NOS: 125-134)-18B(SEQ ID NO: 108), 19A(SEQ ID NOS: 135-144)-19B(SEQ ID NO: 145), 20A(SEQ ID NOS: 146-155)-20B(SEQ ID NO: 156), 21A(SEQ ID NOS: 157-166)-21B(SEQ ID NO: 167), 22A(22A SEQ ID NOS: 168-177)-22B(SEQ ID NO: 178), and/or 23A(SEQ ID NOS: 179-188)-23B(SEQ ID NO: 189).

10. The kit of aspect 8, wherein the one or more primer sets is/are configured to amplify a region of any 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more consecutive nucleotides in a polynucleotide that is 80, 81, 82, 83, 84, 85, 86, 87, 88, 89, 90, 91, 92, 93, 94, 95, 96, 97, 98, 99, to/or 100 percent identical to any one or more of SEQ ID NOs: 1-8, 97-108, 145, 156, 167, 178, 189-196 or any sequences complementary thereto.

11. An assay to detect one or more shrimp or fish pathogens comprising:
amplifying one or more shrimp or fish pathogen-specific target polynucleotides in a sample using loop-mediate isothermal amplification (LAMP), wherein amplification of the one or more shrimp or fish pathogen-specific target nucleotides generates a detectable signal; and
detecting and/or measuring the detectable signal thereby determining the presence or absence of a shrimp or fish pathogen.
   12. The assay of aspect 11, wherein the detectable signal is produced by a pH sensitive reagent that changes color from a first visible color to a second visible color in response to amplification of a target polynucleotide.
   13. The assay of aspect 12, wherein the first visible color and the second visible color are different colors.
   14. The assay of any one of aspects 12-13, wherein the pH sensitive indicator changes color when the LAMP amplification moves from a neutral or basic pH to an acid pH as the reaction proceeds.
   15. The assay of any one of aspects 12-14, wherein the pH sensitive indicator is phenol red, cresol red, phenolphthalein, methyl orange, thymol blue, bromothymol blue, or neutral red, or m-cresol purple.
   16. The assay of any one of aspects 11-15, wherein the detectable signal is visible to the naked eye.
   17. The assay of any one of aspects 11-16, wherein detecting and/or measuring comprises imaging or otherwise analyzing the LAMP reaction with a smartphone, tablet, or other portable electronic device.
   18. The assay of any one of aspects 11-17, wherein LAMP is performed using one or more primer sets configured for LAMP amplification and specific for one or more the shrimp or fish pathogens selected from: White Spot Syndrome Virus (WSSV). Enterocytozoon hepatopenaei (EHP). Necrotising Hepatopancreatitis/Hepatobacter penaei (NHP-B). Infectious Hypodermal and Hematopoietic Necrosis Virus (IHHNV). Infectious Myonecrosis Virus (IMNV). Vibrio spp.-causing Acute hepatopancreatic necrosis disease (AHPND)/EMS). Yellow Head Virus (YHV). Taura Syndrome Virus (TSV). Vibrio parahaemolyticus. Tilapia Lake virus (TiLV), or any combination thereof.
   19. The assay of aspect 18, wherein one or more primers of the one or more primer sets is selected from one or more of those set forth or otherwise identified in any one or more of Tables 1 (SEQ ID NOs: 9-14), 3 (SEQ ID NOS: 17-24), 4 (SEQ ID NOS: 25-32), 5 (SEQ ID NOS: 33-40), 6 (SEQ ID NOS: 41-48), 7 (SEQ ID NOS: 49-56), 8 (SEQ ID NOS: 57-64), 9 (SEQ ID NOS: 65-72), 10 (SEQ ID NOS: 73-80), 11A (SEQ ID NOS: 81-88), 11B (SEQ ID NOS: 125-126, 129-134), 12 (SEQ ID NOS: 89-96), 13 (SEQ ID NOS: 135-136, 139-144), 14 (SEQ ID NOS: 146-147, 150-155), 15 (SEQ ID NOS: 157-158, 161-166), 16 (SEQ ID NOS: 168-169, 172-177), and/or 17 (SEQ ID NOS: 179-180, 183-188) and/or FIGS. 8A(SEQ ID NOS: 17-24)-8B(SEQ ID NO: 97), 9A(SEQ ID NOS: 65-72)-9B(SEQ ID NO: 98):. 10A(SEQ ID NOS: 33-40)-10B(SEQ ID NO: 99), 11A(SEQ ID NOS: 49-56)-11B(SEQ ID NO: 100), 12A(SEQ ID NOS: 81-88, 101-102)-12B(SEQ ID NO: 103), 13A(SEQ ID NOS: 25-27, 29-32, 38)-13B(SEQ ID NO: 104), 14A(SEQ ID NOS: 89-96)-14B(SEQ ID NO: 105), and 15A(SEQ ID NOS: 109-116)-15B(SEQ ID NO: 106), 16A(SEQ ID NOS: 117-124)-16B(SEQ ID NO: 107), 17A(SEQ ID NOS: 73-80)-17B(SEQ ID NO: 196), 18A(SEQ ID NOS: 125-134)-18B(SEQ ID NO: 108), 19A(SEQ ID NOS: 135-144)-19B(SEQ ID NO: 145), 20A(SEQ ID NOS: 146-155)-20B(SEQ ID NO: 156), 21A(SEQ ID NOS: 157-166)-21B(SEQ ID NO: 167), 22A(22A SEQ ID NOS: 168-177)-22B(SEQ ID NO: 178), and/or 23A(SEQ ID NOS: 179-188)-23B(SEQ ID NO: 189).
   20). The assay of aspect 18, wherein the one or more primer sets is/are configured to amplify a region of any 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more consecutive nucleotides in a polynucleotide that is 80, 81, 82, 83, 84, 85, 86, 87, 88, 89, 90, 91, 92, 93, 94, 95, 96, 97, 98, 99, to/or 100 percent identical to any one or more of SEQ ID NOs: 1-8, 97-108, 145, 156, 167, 178, 189-196 or any sequence complementary thereto.

What is claimed is:

1. A kit configured to detect one or more shrimp or fish pathogens using loop-mediate isothermal amplification (LAMP), the kit comprising:
one or more primer sets configured to specifically amplify one or more shrimp or fish pathogen-specific target polynucleotides via LAMP; and
one or more amplification reagents.

2. The kit of claim 1, further comprising one or more signal reagents.

3. The kit of claim 2, wherein at least one of the one or more signal reagents is a pH sensitive reagent that is capable of changing color from a first visible color to a second visible color in response to amplification of a target polynucleotide.

4. The kit of claim 3, wherein the first visible color and the second visible color are different colors.

5. The kit of claim 3, wherein the pH sensitive indicator changes color when the LAMP amplification moves from a neutral or basic pH to an acid pH as the reaction proceeds.

6. The kit of claim 3, wherein the pH sensitive indicator is phenol red, cresol red, phenolphthalein, methyl orange, thymol blue, bromothymol blue, or neutral red, or m-cresol purple.

7. The kit of claim 3, wherein the signal reagent is capable of producing a signal detectable by the naked eye, an optical sensor, a camera, a smartphone, a tablet, an electronic portable device, or any combination thereof.

8. The kit of claim 1, wherein the one or more primer sets configured for LAMP amplification is/are specific for one or more the shrimp or fish pathogens selected from: White Spot Syndrome Virus (WSSV), Enterocytozoon hepatopenaei (EHP), Necrotising Hepatopancreatitis/Hepatobacter penaei (NHP-B), Infectious Hypodermal and Hematopoietic Necrosis Virus (IHHNV), Infectious Myonecrosis Virus (IMNV), Vibrio spp.-causing Acute hepatopancreatic necrosis disease (AHPND)/EMS), Yellow Head Virus (YHV), Taura Syndrome Virus (TSV), Vibrio parahaemolyticus, Tilapia Lake virus (TiLV), or any combination thereof.

9. The kit of claim 8, wherein one or more primers of the one or more primer sets is selected from one or more of those set forth or otherwise identified in any one or more of Tables 1 (SEQ ID NOs: 9-14), 3 (SEQ ID NOS: 17-24), 4 (SEQ ID NOS: 25-32), 5 (SEQ ID NOS: 33-40), 6 (SEQ ID NOS: 41-48), 7 (SEQ ID NOS: 49-56), 8 (SEQ ID NOS: 57-64), 9 (SEQ ID NOS: 65-72), 10 (SEQ ID NOS: 73-80), 11A (SEQ ID NOS: 81-88), 11B (SEQ ID NOS: 125-126, 129-134), 12 (SEQ ID NOS: 89-96), 13 (SEQ ID NOS: 135-136, 139-144), 14 (SEQ ID NOS: 146-147, 150-155), 15 (SEQ ID NOS: 157-158, 161-166), 16 (SEQ ID NOS: 168-169, 172-177), and/or 17 (SEQ ID NOS: 179-180, 183-188) and/or FIGS. 8A(SEQ ID NOS: 17-24)-8B(SEQ ID NO: 97), 9A(SEQ ID NOS: 65-72)-9B(SEQ ID NO: 98):, 10A(SEQ ID NOS: 33-40)-10B(SEQ ID NO: 99), 11A(SEQ ID NOS: 49-56)-11B(SEQ ID NO: 100), 12A(SEQ ID NOS: 81-88, 101-102)-12B(SEQ ID NO: 103), 13A(SEQ ID NOS: 25-27, 29-32, 38)-13B(SEQ ID NO: 104), 14A(SEQ ID NOS: 89-96)-14B(SEQ ID NO: 105), and 15A(SEQ ID NOS: 109-116)-15B(SEQ ID NO: 106), 16A(SEQ ID NOS: 117-124)-16B(SEQ ID NO: 107), 17A(SEQ ID NOS: 73-80)-17B(SEQ ID NO: 196), 18A(SEQ ID NOS: 125-134)-18B(SEQ ID NO: 108), 19A(SEQ ID NOS: 135-144)-19B(SEQ ID NO: 145), 20A(SEQ ID NOS: 146-155)-20B(SEQ ID NO: 156), 21A(SEQ ID NOS: 157-166)-21B(SEQ ID NO: 167), 22A(22A SEQ ID NOS: 168-177)-22B(SEQ ID NO: 178), and/or 23A(SEQ ID NOS: 179-188)-23B(SEQ ID NO: 189).

10. The kit of claim 8, wherein the one or more primer sets is/are configured to amplify a region of any 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more consecutive nucleotides in a polynucleotide that is 80, 81, 82, 83, 84, 85, 86, 87, 88, 89, 90, 91, 92, 93, 94, 95, 96, 97, 98, 99, to/or 100 percent identical to any one or more of SEQ ID NOs: 1-8, 97-108, 145, 156, 167, 178, 189-196 or any sequences complementary thereto.

11. An assay to detect one or more shrimp or fish pathogens comprising:
amplifying one or more shrimp or fish pathogen-specific target polynucleotides in a sample using loop-mediate isothermal amplification (LAMP), wherein amplification of the one or more shrimp or fish pathogen-specific target nucleotides generates a detectable signal; and
detecting and/or measuring the detectable signal thereby determining the presence or absence of a shrimp or fish pathogen.

12. The assay of claim 11, wherein the detectable signal is produced by a pH sensitive reagent that changes color from a first visible color to a second visible color in response to amplification of a target polynucleotide.

13. The assay of claim 12, wherein the first visible color and the second visible color are different colors.

14. The assay of claim 12, wherein the pH sensitive indicator changes color when the LAMP amplification moves from a neutral or basic pH to an acid pH as the reaction proceeds.

15. The assay of claim 12, wherein the pH sensitive indicator is phenol red, cresol red, phenolphthalein, methyl orange, thymol blue, bromothymol blue, or neutral red, or m-cresol purple.

16. The assay of claim 11, wherein the detectable signal is visible to the naked eye.

17. The assay of any one of claims 11-16, wherein detecting and/or measuring comprises imaging or otherwise analyzing the LAMP reaction with a smartphone, tablet, or other portable electronic device.

18. The assay of any one of claims 11-17, wherein LAMP is performed using one or more primer sets configured for LAMP amplification and specific for one or more the shrimp or fish pathogens selected from: White Spot Syndrome Virus (WSSV), Enterocytozoon hepatopenaei (EHP), Necrotising Hepatopancreatitis/Hepatobacter penaei (NHP-B), Infectious Hypodermal and Hematopoietic Necrosis Virus (IHHNV), Infectious Myonecrosis Virus (IMNV), Vibrio spp.-causing Acute hepatopancreatic necrosis disease (AHPND)/EMS), Yellow Head Virus (YHV), Taura Syndrome Virus (TSV), Vibrio parahaemolyticus, Tilapia Lake virus (TILV), or any combination thereof.

19. The assay of claim 18, wherein one or more primers of the one or more primer sets is selected from one or more of those set forth or otherwise identified in any one or more of Tables 1 (SEQ ID NOs: 9-14), 3 (SEQ ID NOS: 17-24), 4 (SEQ ID NOS: 25-32), 5 (SEQ ID NOS: 33-40), 6 (SEQ ID NOS: 41-48), 7 (SEQ ID NOS: 49-56), 8 (SEQ ID NOS: 57-64), 9 (SEQ ID NOS: 65-72), 10 (SEQ ID NOS: 73-80), 11A (SEQ ID NOS: 81-88), 11B (SEQ ID NOS: 125-126, 129-134), 12 (SEQ ID NOS: 89-96), 13 (SEQ ID NOS: 135-136, 139-144), 14 (SEQ ID NOS: 146-147, 150-155), 15 (SEQ ID NOS: 157-158, 161-166), 16 (SEQ ID NOS: 168-169, 172-177), and/or 17 (SEQ ID NOS: 179-180, 183-188) and/or FIGS. 8A(SEQ ID NOS: 17-24)-8B(SEQ ID NO: 97), 9A(SEQ ID NOS: 65-72)-9B(SEQ ID NO: 98):, 10A(SEQ ID NOS: 33-40)-10B(SEQ ID NO: 99), 11A(SEQ ID NOS: 49-56)-11B(SEQ ID NO: 100), 12A(SEQ ID NOS: 81-88, 101-102)-12B(SEQ ID NO: 103), 13A(SEQ ID NOS: 25-27, 29-32, 38)-13B(SEQ ID NO: 104), 14A(SEQ ID NOS: 89-96)-14B(SEQ ID NO: 105), and 15A(SEQ ID NOS: 109-116)-15B(SEQ ID NO: 106), 16A(SEQ ID NOS: 117-124)-16B(SEQ ID NO: 107), 17A(SEQ ID NOS: 73-80)-17B(SEQ ID NO: 196), 18A(SEQ ID NOS: 125-134)-18B(SEQ ID NO: 108), 19A(SEQ ID NOS: 135-144)-19B(SEQ ID NO: 145), 20A(SEQ ID NOS: 146-155)-20B(SEQ ID NO: 156), 21A(SEQ ID NOS: 157-166)-21B(SEQ ID NO: 167), 22A(22A SEQ ID NOS: 168-177)-22B(SEQ ID NO: 178), and/or 23A(SEQ ID NOS: 179-188)-23B(SEQ ID NO: 189).

20. The assay of claim 18, wherein the one or more primer sets is/are configured to amplify a region of any 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 or more consecutive nucleotides in a polynucleotide that is 80, 81, 82, 83, 84, 85, 86, 87, 88, 89, 90, 91, 92, 93, 94, 95, 96, 97, 98, 99, to/or 100 percent identical to any one or more of SEQ ID NOs: 1-8, 97-108, 145, 156, 167, 178, 189-196 or any sequence complementary thereto.
